SEQUENCE LISTING (1 sequence)

SEQ ID NO: 1 - protein, 15 aa, synthetic construct
  REGION 1..15: note = Biotin-labeled peptide, custom synthesis
EQEDEPEGDYFEWLE

(19) United States
(12) Patent Application Publication
(10) Pub. No.: US 2025/0304582 A1
(43) Pub. Date: Oct. 2, 2025

(54) Bicyclic Ureas As Kinase Inhibitors

(71) Applicant: Incyte Corporation, Wilmington, DE (US)

(72) Inventors: Hang Thi Dang, Wilmington, DE (US); Charles Cole, Wilmington, DE (US); Javier A. Feliciano Leal, Bryn Mawr, PA (US); Peng He, Wilmington, DE (US); Chunhong He, Boothwyn, PA (US); Xin Li, Claymont, DE (US); Michael Liang, Wilmington, DE (US); Cooper Ashley Taylor, Wilmington, DE (US); Oleg Vechorkin, Wilmington, DE (US); Eddy W. Yue, Landenberg, PA (US)

(21) Appl. No.: 19/092,769

(22) Filed: Mar. 27, 2025

Related U.S. Application Data
(60) Provisional application No. 63/571,388, filed on Mar. 28, 2024.

(51) Int. Cl. C07D 471/04; A61K 31/4375; A61K 31/4709; A61K 31/498; A61K 31/5025; C07D 519/00

(52) U.S. Cl. CPC C07D 471/04; A61K 31/4375; A61K 31/4709; A61K 31/498; A61K 31/5025; C07D 519/00

(57) ABSTRACT

The present application provides bicyclic urea compounds that modulate the activity of JAK2, which are useful in the treatment of various diseases, including cancer.

### SEQUENCE LISTING

This application contains a Sequence Listing that has been submitted electronically as an XML file named “20443-0847001_SL_ST26.XML.” The XML file, created on Mar. 27, 2025, is 2,281 bytes in size. The material in the XML file is hereby incorporated by reference in its entirety.

### TECHNICAL FIELD

The present invention provides bicyclic urea compounds that modulate the activity of the JAK2 and are useful in the treatment of diseases related to JAK2, including cancer.

### BACKGROUND

Janus kinase (JAK) 2 plays pivotal roles in signaling by several cytokine receptors. The mutant JAK2 V617F, located at pseudokinase (JH2) domain, is the most common molecular event associated with myeloproliferative neoplasms (MPNs). Current JAK2 small molecule inhibitors used to treat MPNs are designed to target the JAK2 kinase (JH1) domain. Thus, selective targeting of the JAK2 V617F mutant over the JAK2 kinase (JH1) domain may be useful for treating various pathologies, while sparing essential JAK2 functions. This application is directed to this need and others.

### SUMMARY

The present invention relates to, inter alia, compounds of Formula I.

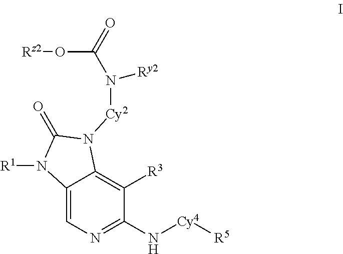

or pharmaceutically acceptable salts thereof, wherein constituent members are defined herein.

The present invention further provides pharmaceutical compositions comprising a compound of Formula I, or a pharmaceutically acceptable salt thereof, and a pharmaceutically acceptable carrier.

The present invention further provides methods of inhibiting an activity of the V617F variant of JAK2 kinase comprising contacting the kinase with a compound of Formula I, or a pharmaceutically acceptable salt thereof.

The present invention further provides methods of treating a disease or a disorder associated with expression or activity of the V617F variant of JAK2 kinase in a patient by administering to a patient a therapeutically effective amount of a compound of Formula I, or a pharmaceutically acceptable salt thereof.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DETAILED DESCRIPTION

The present application provides a compound of Formula I:

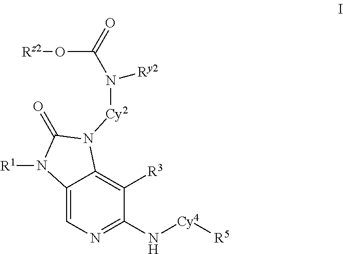

or a pharmaceutically acceptable salt thereof, wherein:
R¹ is selected from C₁₋₆ alkyl, C₂₋₆ alkenyl, and C₂₋₆ alkynyl, wherein the C₁₋₆ alkyl, C₂₋₆ alkenyl, and C₂₋₆ alkynyl of R¹ are each optionally substituted with 1, 2, or 3 independently selected R^{1A} substituents;
each R^{1A} is independently selected from halo, oxo, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11};
each R^{a11}, R^{b11}, R^{c11}, and R^{d11} is independently selected from H, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{a11}, R^{b11}, R^{c11} and R^{d11} are each optionally substituted with 1, 2, 3, 4, 5, 6, 7, or 8 independently selected R^{M} substituents;
each R^{c11} is independently selected from H, OH, CN, C₁₋₆ alkyl, C₁₋₆ alkoxy, C₁₋₆ haloalkyl, C₁₋₆ haloalkoxy, C₂₋₆ alkenyl, and C₂₋₆ alkynyl;
Cy² is selected from C₃₋₁₀ cycloalkyl and 4-12 membered heterocycloalkyl, wherein the C₃₋₁₀ cycloalkyl and 4-12 membered heterocycloalkyl are each optionally substituted with 1, 2, 3, 4, 5, 6, 7 or 8 independently selected R² substituents;
each R² is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})R^{c2}R^{d2}, NR^{c2}C(═NR^{e2})R^{c2}R^{d2}, NR^{c2}C(═NR^{e2})R^{b2}, NR^{c2}S(O)R^{b2}, NR^{c2}S(O)NR^{c2}R^{d2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f2} and R^{g2} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{y2} is selected from H, C₁₋₆ alkyl, C₁₋₆ haloalkyl, C₂₋₆ alkenyl, and C₂₋₆ alkynyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, and (4-10 membered heterocycloalkyl)-C₁₋₆ alkyl- of R^{z2} are each optionally substituted with 1, 2, 3, 4, 5, 6, 7, or 8 independently selected R^{2A} substituents;
each R^{2A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f21} and R^{g21} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h21} and Rⁱ²¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)R^{b22}, NR^{c22}S(O)NR^{c22}R^{d22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22})R^{b22}, and OS(O)₂R^{b22}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{2C} are each optionally substituted with 1, 2, 3, or 4 independently selected R^{M} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-of R^{a22}, R^{c22} and R^{d22} are each optionally substituted with 1, 2, 3, or 4 independently selected R^{M} substituents;
or, any R^{c22} and R^{d22} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b22} are each optionally substituted with 1, 2, 3, or 4 independently selected R^{M} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R³ is selected from H, halo, C₁₋₆ alkyl, C₂₋₆ alkenyl, and C₂₋₆ alkynyl;
Cy⁴ is selected from 8-11 membered bicyclic heteroaryl, wherein the 8-11 membered bicyclic heteroaryl is optionally substituted with 1, 2, 3, 4, 5, 6, 7 or 8 independently selected R⁴ substituents;
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆alkyl-, (5-10 membered heteroaryl)-C₁₋₆ alkyl-, CN, NO₂, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4} S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R⁴ are each optionally substituted with 1, 2, 3, 4, 5, 6, 7, or 8 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{a4}, R^{c4} and R^{d4} are each optionally substituted with 1, 2, 3, 4, 5, 6, 7, or 8 independently selected R^{4A} substituents;
or, any R^{c4} and R^{d4} attached to the same N atom, together with the N atom to which they are attached, form a 5-10 membered heteroaryl, wherein the 5-10 membered heteroaryl is optionally substituted with 1, 2, 3, or 4 independently selected R^{4A} substituents;
each R^{b4} is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{b4} are each optionally substituted with 1, 2, 3, 4, 5, 6, 7, or 8 independently selected R^{4A} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{j4} and R^{k4} is independently selected from OH, C₁₋₆ alkoxy, and C₁₋₆ haloalkoxy;
or any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4A} is independently selected from halo, oxo, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆alkyl-, (5-10 membered heteroaryl)-C₁₋₆ alkyl-, CN, NO₂, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, SF₅, P(O)R^{f41}R^{g41}, OP(O)(OR^{h41})(ORⁱ⁴¹), P(O)(OR^{h41})(ORⁱ⁴¹), and BR^{j41}R^{k41}, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{4A} are each optionally substituted with 1, 2, 3, 4, 5, 6, 7, or 8 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{a41}, R^{c41} and R^{d41} are each optionally substituted with 1, 2, 3, 4, 5, 6, 7, or 8 independently selected R^{4B} substituents;
or, any R^{c41} and R^{d41} attached to the same N atom, together with the N atom to which they are attached, form a 5-10 membered heteroaryl, wherein the 5-10 membered heteroaryl is optionally substituted with 1, 2, 3, or 4 independently selected R^{4B} substituents;
each R^{b41} is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{b41} are each optionally substituted with 1, 2, 3, 4, 5, 6, 7, or 8 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{f41} and R^{g41} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{h41} and Rⁱ⁴¹ is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{j41} and R^{k41} is independently selected from OH, C₁₋₆ alkoxy, and C₁₋₆ haloalkoxy;
or any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from halo, oxo, C₁₋₆ alkyl, C₁₋₆ haloalkyl, C₂₋₆ alkenyl, C₂₋₆ alkynyl, phenyl, 5-6 membered heteroaryl, phenyl-C₁₋₆ alkyl-, (5-6 membered heteroaryl)-C₁₋₆ alkyl-, CN, NO₂, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)R^{b42}, NR^{c42}S(O)NR^{c42}R^{d42}, NR^{c42}S(O)₂R^{b42}, NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, and OS(O)₂R^{b42}, wherein the C₁₋₆ alkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl- of R^{4B} are each optionally substituted with 1, 2, 3, or 4 independently selected R^{P} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl-, wherein the C₁₋₆ alkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl- of R^{a42}, R^{c42} and R^{d42} are each optionally substituted with 1, 2, 3, or 4 independently selected R^{P} substituents;
or, any R^{c42} and R^{d42} attached to the same N atom, together with the N atom to which they are attached, form a 5-6 membered heteroaryl, wherein the 5-6 membered heteroaryl is optionally substituted with 1, 2, 3, or 4 independently selected R^{P} substituents;
each R^{b42} is independently selected from H, C₁₋₆ alkyl, C₁₋₆ haloalkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl-, wherein the C₁₋₆ alkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl- of R^{b42} are each optionally substituted with 1, 2, 3, or 4 independently selected R^{P} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl-;
R⁵ is selected from —C₁₋₆ alkyl-(R^{a51})ₚ, halo, CN, NO₂, oxo, OR^{b51}, SR^{b51}, NR^{c51}R^{b51}, OC(O)R^{b51}, OC(NR^{c51})R^{b51}, OC(O)NR^{c51}R^{b51}, NR^{c51}C(O)R^{b51}, NR^{c51}C(O)OR^{b51}, NR^{c51}C(O)NR^{c51}R^{b51}, NR^{c51}SO₂R^{b51}, NR^{c51}SO₂NR^{c51}R^{b51}, NR^{c51}S(O)R^{b51}, C(O)OR^{b51}, C(O)R^{b51}, C(O)NR^{c51}R^{b51}, S(O)R^{b51}, S(O)NR^{c51}R^{b51}, S(O)₂R^{b51}, S(O)(NH)R^{b51}, and SO₂NR^{c51}R^{b51};
p is 0, 1, 2, 3, 4, 5, or 6;
each R^{a51} is independently selected from halo, CN, NO₂, oxo, OR^{a52}, NHR^{a52}, NR^{b52}R^{a52}, SR^{a52}, SO₂R^{a52}, C(O)R^{a52}, C(O)OR^{a52}, C(O)NHR^{a52}, C(O)NR^{b52}R^{a52}, OC(O)R^{a52}, S(O)R^{a52}, S(O)NR^{b52}R^{a52}, S(O)₂R^{a52}, and SO₂NR^{b52}R^{a52};
each R^{b51} and R^{c51} are independently selected from H, C₁₋₆ alkyl, and C₁₋₆ haloalkyl;
each R^{a52} and R^{b52} are independently selected from H, and C₁₋₆ alkyl;
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-; and
each R^{P} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-.

In some embodiments, R¹ is C₁₋₆ alkyl.

In some embodiments, R¹ is C₁₋₃ alkyl.

In some embodiments, R¹ is selected from methyl and trideuteromethyl.

In some embodiments, R¹ is methyl.

In some embodiments, R¹ is trideuteromethyl.

In some embodiments, Cy² is selected from C₃₋₁₀ cycloalkyl and 4-12 membered heterocycloalkyl, wherein the C₃₋₁₀ cycloalkyl and 4-12 membered heterocycloalkyl are each optionally substituted with 1, 2, 3, or 4 independently selected R² substituents.

In some embodiments, Cy² is selected from C₃₋₁₀ cycloalkyl and 4-10 membered heterocycloalkyl, wherein the C₃₋₁₀ cycloalkyl and 4-10 membered heterocycloalkyl are each optionally substituted with 1, 2, 3, or 4 independently selected R² substituents.

In some embodiments, Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents.

In some embodiments, Cy² is C₃₋₁₀ cycloalkyl, wherein the C₃₋₁₀ cycloalkyl is optionally substituted with 1, 2, 3, or 4 independently selected R² substituents.

In some embodiments, Cy² is monocyclic C₃₋₁₀ cycloalkyl or a bicyclic C₃₋₁₀ cycloalkyl, wherein the monocyclic C₃₋₁₀ cycloalkyl and bicyclic C₃₋₁₀ cycloalkyl are each optionally substituted with 1, 2, 3, or 4 independently selected R² substituents.

In some embodiments, Cy² is C₃₋₇ cycloalkyl, wherein the C₃₋₇ cycloalkyl is optionally substituted with 1, 2, 3, or 4 independently selected R² substituents.

In some embodiments, Cy² is monocyclic C₃₋₇ cycloalkyl or a bicyclic C₃₋₇ cycloalkyl, wherein the monocyclic C₃₋₇ cycloalkyl and bicyclic C₃₋₇ cycloalkyl are each optionally substituted with 1, 2, 3, or 4 independently selected R² substituents.

In some embodiments, Cy² is selected from cyclobutyl, cyclopentyl, deuterocyclopentyl, bicyclo[1.1.1]pentanyl, and bicyclo[2.1.1]hexanyl, wherein the cyclobutyl, cyclopentyl, deuterocyclopentyl, bicyclo[1.1.1]pentanyl, and bicyclo[2.1.1]hexanyl of Cy² are each optionally substituted with 1, 2, 3, or 4 independently selected R² substituents.

In some embodiments, Cy² is selected from cyclobutyl, cyclopentyl, bicyclo[1.1.1]pentanyl, and bicyclo[2.1.1]hexanyl, wherein the cyclobutyl, cyclopentyl, bicyclo[1.1.1]pentanyl, and bicyclo[2.1.1]hexanyl of Cy² are each optionally substituted with 1, 2, 3, or 4 independently selected R² substituents.

In some embodiments, Cy² is cyclobutyl, which is optionally substituted with 1, 2, 3, or 4 independently selected R² substituents.

In some embodiments, Cy² is cyclopentyl, which is optionally substituted with 1, 2, 3, or 4 independently selected R² substituents.

In some embodiments, Cy² is deuterocyclopentyl, which is optionally substituted with 1, 2, 3, or 4 independently selected R² substituents.

In some embodiments, Cy² is bicyclo[1.1.1]pentanyl, which is optionally substituted with 1, 2, 3, or 4 independently selected R² substituents.

In some embodiments, Cy² is bicyclo[2.1.1]hexanyl, which is optionally substituted with 1, 2, 3, or 4 independently selected R² substituents.

In some embodiments, Cy² is selected from:

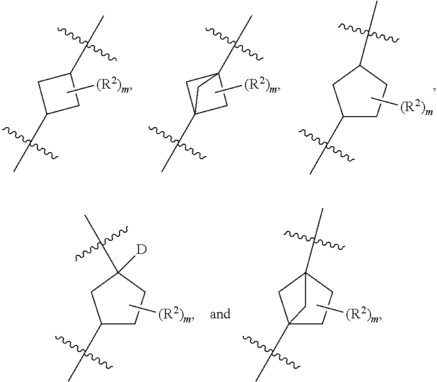

wherein m is 0, 1, 2, 3, or 4.

In some embodiments, Cy² is selected from:

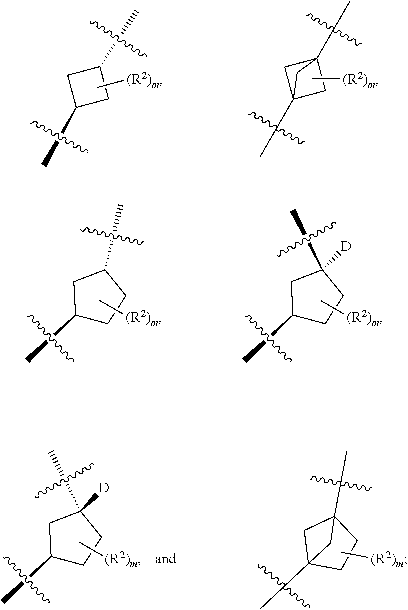

wherein m is 0, 1, 2, 3, or 4.

In some embodiments, each R² is independently selected from halo, oxo, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{2A} substituents.

In some embodiments, each R² is independently selected from halo, oxo, C₁₋₆ alkyl, C₁₋₆ haloalkyl, C₂₋₆ alkenyl, and C₂₋₆ alkynyl.

In some embodiments, each R² is independently selected from C₁₋₆ alkyl, C₁₋₆ haloalkyl, C₂₋₆ alkenyl, and C₂₋₆ alkynyl.

In some embodiments, each R² is independently selected from C₁₋₆ alkyl.

In some embodiments, each R² is independently selected from C₁₋₃ alkyl.

In some embodiments, each R² is methyl.

In some embodiments, Cy² is selected from cyclobutyl, cyclopentyl, deuterocyclopentyl, methylcyclopentyl, bicyclo[1.1.1]pentanyl, and bicyclo[2.1.1]hexanyl.

In some embodiments, Cy² is selected from cyclobutyl, cyclopentyl, methylcyclopentyl, bicyclo[1.1.1]pentanyl, and bicyclo[2.1.1]hexanyl.

In some embodiments, Cy² is selected from:

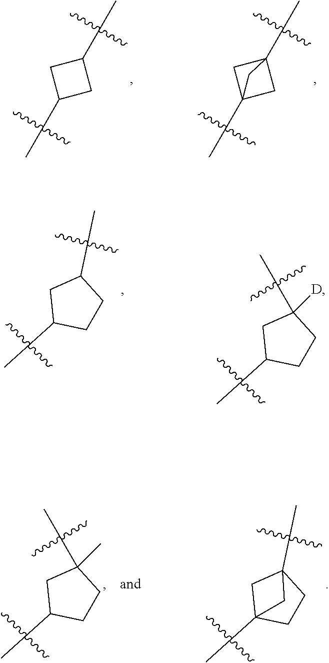

In some embodiments, Cy² is selected from:

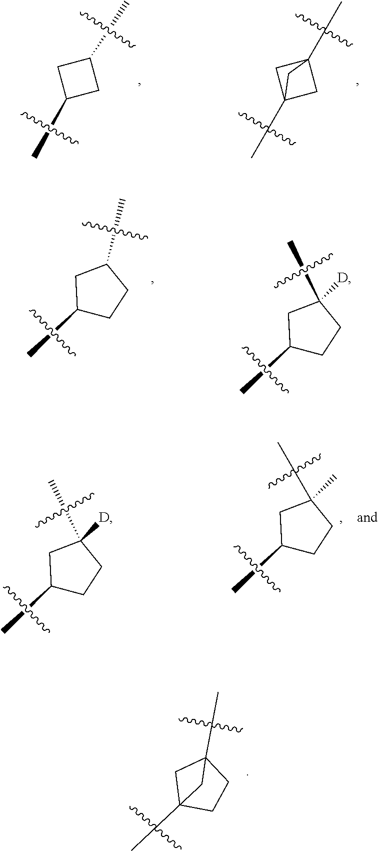

In some embodiments, R^{y2} is selected from H and C₁₋₆ alkyl.

In some embodiments, R^{y2} is H.

In some embodiments, R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents.

In some embodiments, R^{z2} is selected from C₁₋₆ alkyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents.

In some embodiments, R^{z2} is selected from C₁₋₆ alkyl, phenyl, C₃₋₇ cycloalkyl, phenyl-C₁₋₆ alkyl-, and C₃₋₇ cycloalkyl-C₁₋₆ alkyl-, wherein the C₁₋₆ alkyl, phenyl, C₃₋₇ cycloalkyl, phenyl-C₁₋₆ alkyl-, and C₃₋₇ cycloalkyl-C₁₋₆ alkyl- of R^{z2} are each optionally substituted with 1, 2, 3, or 4 independently selected R^{2A} substituents.

In some embodiments, R^{z2} is selected from methyl, ethyl, cyclopropyl, and phenylmethyl, wherein the methyl, ethyl, cyclopropyl, and phenylmethyl of R^{z2} are each optionally substituted with 1, 2, 3, or 4 independently selected R^{2A} substituents.

In some embodiments, each R^{2A} is independently selected from C₁₋₆ alkyl, C₁₋₆ haloalkyl, C₂₋₆ alkenyl, C₂₋₆ alkynyl, and OR^{a21}.

In some embodiments, each R^{2A} is independently selected from OR^{a21}.

In some embodiments, each R^{a21}, R^{b21}, R^{c21}, and R^{d21} is independently selected from H and C₁₋₆ alkyl.

In some embodiments, each R^{a21} is independently selected from H and C₁₋₆ alkyl.

In some embodiments, each R^{a21} is independently selected from H and C₁₋₃ alkyl.

In some embodiments, each R^{a21} is independently selected from C₁₋₆ alkyl.

In some embodiments, each R^{a21} is independently selected from C₁₋₃ alkyl.

In some embodiments, each R^{a21} is methyl.

In some embodiments, each R^{2A} is independently selected from C₁₋₆ alkyl, C₁₋₆ haloalkyl, C₂₋₆ alkenyl, C₂₋₆ alkynyl, and OR^{a21}; and
each R^{a21} is independently selected from H and C₁₋₆ alkyl.

In some embodiments, each R^{2A} is independently selected from OR^{a21}; and
each R^{a21} is independently selected from H and C₁₋₃ alkyl.

In some embodiments, each R^{2A} is methoxy.

In some embodiments, R^{z2} is selected from methyl, methoxyethyl, cyclopropyl, and phenylmethyl.

In some embodiments, R^{z2} is methyl.

In some embodiments, R^{z2} is methoxyethyl.

In some embodiments, R^{z2} is cyclopropyl.

In some embodiments, R^{z2} is phenylmethyl.

In some embodiments, R³ is selected from H and C₁₋₆ alkyl.

In some embodiments, R³ is H.

In some embodiments, Cy⁴ is a 8-10 membered bicyclic heteroaryl, wherein the 8-10 membered bicyclic heteroaryl is optionally substituted with 1, 2, 3, or 4 independently selected R⁴ substituents.

In some embodiments, Cy⁴ is a 9-10 membered bicyclic heteroaryl, wherein the 9-10 membered bicyclic heteroaryl is optionally substituted with 1, 2, 3, or 4 independently selected R⁴ substituents.

In some embodiments, Cy⁴ is selected from quinolinyl, naphthyridinyl, quinoxalinyl, and imidazo[1,2-b]pyridazinyl, wherein the quinolinyl, naphthyridinyl, quinoxalinyl, and imidazo[1,2-b]pyridazinyl are each optionally substituted with 1, 2, 3, or 4 independently selected R⁴ substituents.

In some embodiments, Cy⁴ is quinolinyl, wherein the quinolinyl is optionally substituted with 1, 2, 3, or 4 independently selected R⁴ substituents.

In some embodiments, Cy⁴ is naphthyridinyl, wherein the naphthyridinyl is optionally substituted with 1, 2, 3, or 4 independently selected R⁴ substituents.

In some embodiments, Cy⁴ is quinoxalinyl, wherein the quinoxalinyl is optionally substituted with 1, 2, 3, or 4 independently selected R⁴ substituents.

In some embodiments, Cy⁴ is imidazo[1,2-b]pyridazinyl, wherein the imidazo[1,2-b]pyridazinyl is optionally substituted with 1, 2, 3, or 4 independently selected R⁴ substituents.

In some embodiments, Cy⁴ is selected from:

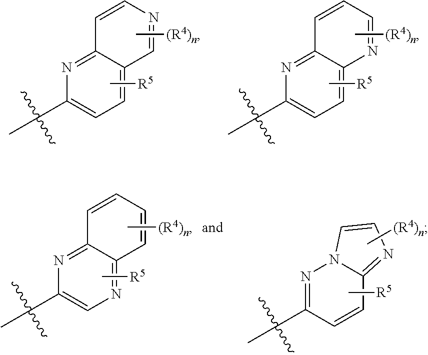

wherein n is 0, 1, 2, or 3.

In some embodiments, Cy⁴ is selected from:

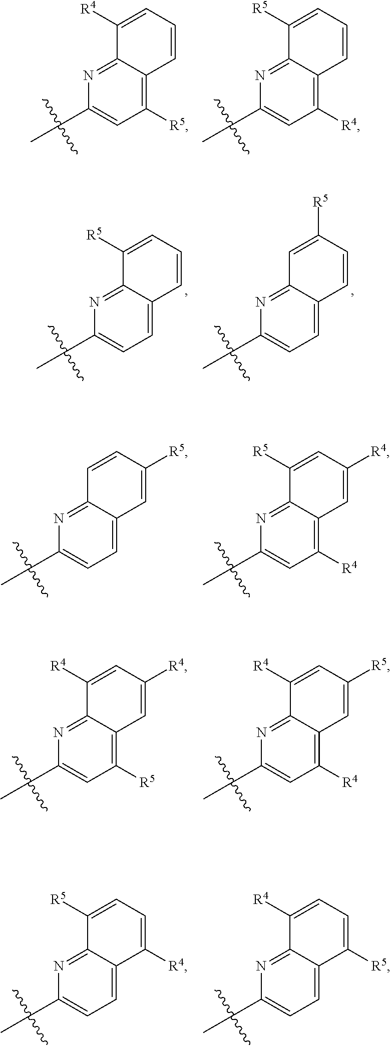

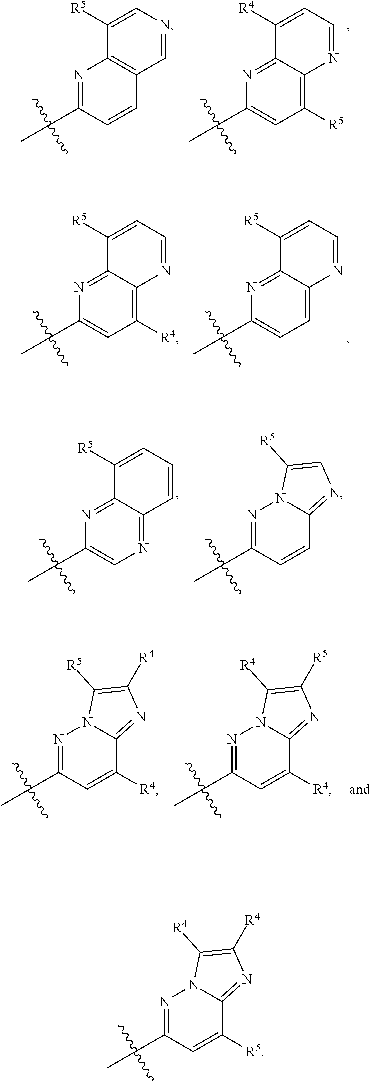

In some embodiments, each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4}, wherein each C₁₋₆ alkyl of R⁴ is optionally substituted with 1 or 2 independently selected R^{4A} substituents.

In some embodiments, each R⁴ is independently selected from C₁₋₆ alkyl, C₁₋₆ haloalkyl, and NR^{c4}C(O)R^{b4}, wherein each C₁₋₆ alkyl of R⁴ is optionally substituted with 1 or 2 independently selected R^{4A} substituents.

In some embodiments, each R⁴ is independently selected from C₁₋₆ alkyl, C₁₋₆ haloalkyl, and NHC(O)R^{b4}, wherein each C₁₋₆ alkyl of R⁴ is optionally substituted with 1 or 2 independently selected R^{4A} substituents.

In some embodiments, each R⁴ is independently selected from methyl, isopropyl, trifluoromethyl, and NR^{c4}C(O)R^{b4}.

In some embodiments, each R⁴ is independently selected from methyl, isopropyl, trifluoromethyl, and NHC(O)R^{b4}.

In some embodiments, each R^{b4} and R^{c4} is independently selected from H, C₁₋₆ alkyl, and C₁₋₆ haloalkyl, wherein the C₁₋₆ alkyl of R^{b4} and R^{c4} are each optionally substituted with 1, 2, 3, or 4 independently selected R^{4A} substituents.

In some embodiments, each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents.

In some embodiments, each R^{b4} is independently selected from H and C₁₋₆ alkyl, wherein the C₁₋₆ alkyl of R^{b4} are each optionally substituted with 1, 2, 3, or 4 independently selected R^{4A} substituents.

In some embodiments, each R^{b4} is independently selected from H and C₁₋₆ alkyl, wherein the C₁₋₆ alkyl of R^{b4} are each optionally substituted with 1, 2, 3, or 4 independently selected R^{4A} substituents; and
each R^{c4} is H.

In some embodiments, each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4}, wherein each C₁₋₆ alkyl, C₂₋₆ alkenyl, and C₂₋₆ alkynyl of R⁴ is optionally substituted with 1 or 2 independently selected R^{4A} substituents; and
each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents.

In some embodiments, each R⁴ is independently selected from C₁₋₆ alkyl, C₁₋₆ haloalkyl, and NHC(O)R^{b4}, wherein each C₁₋₆ alkyl of R⁴ is optionally substituted with 1 or 2 independently selected R^{4A} substituents; and
each R^{b4} is independently selected from H and C₁₋₆ alkyl, wherein each C₁₋₆ alkyl of R^{b4} is optionally substituted with 1 or 2 independently selected R^{4A} substituents.

In some embodiments, each R⁴ is independently selected from methyl, isopropyl, trifluoromethyl, and NHC(O)R^{b4}, wherein each methyl and isopropyl of R⁴ is optionally substituted with 1 or 2 independently selected R^{4A} substituents;
each R^{b4} is independently selected from H and C₁₋₆ alkyl, wherein each C₁₋₆ alkyl of R^{b4} is optionally substituted with 1 or 2 independently selected R^{4A} substituents.

In some embodiments, each R^{4A} is independently selected from C₁₋₆ alkyl, C₁₋₆ haloalkyl, C₂₋₆ alkenyl, C₂₋₆ alkynyl, and OR^{a41}.

In some embodiments, each R^{4A} is independently selected from C₁₋₆ alkyl and OR^{a41}.

In some embodiments, each R^{a41}, R^{b41}, R^{c41}, and R^{d41} is independently selected from H, C₁₋₆ alkyl, and C₁₋₆ haloalkyl.

In some embodiments, each R^{a41}, R^{b41}, R^{c41}, and R^{d41} is independently selected from H and C₁₋₆ alkyl.

In some embodiments, each R^{a41}, R^{b41}, R^{c41}, and R^{d41} is independently selected from H and C₁₋₃ alkyl.

In some embodiments, each R^{a41} is independently selected from H, C₁₋₆ alkyl, and C₁₋₆ haloalkyl.

In some embodiments, each R^{a41} is independently selected from H and C₁₋₆ alkyl.

In some embodiments, each R^{a41} is independently selected from H and C₁₋₃ alkyl.

In some embodiments, each R^{a41} is H.

In some embodiments, each R^{4A} is independently selected from C₁₋₆ alkyl, C₁₋₆ haloalkyl, C₂₋₆ alkenyl, C₂₋₆ alkynyl, and OR^{a41}; and
each R^{a41} is independently selected from H and C₁₋₆ alkyl.

In some embodiments, each R^{4A} is independently selected from C₁₋₆ alkyl and OR^{a41}; and
each R^{a41} is independently selected from H and C₁₋₃ alkyl.

In some embodiments, each R^{4A} is independently selected from methyl and hydroxy.

In some embodiments, each R⁴ is independently selected from methyl, hydroxymethyl, hydroxyisopropyl, trifluoromethyl, and NHC(O)CH₂OCH₃.

In some embodiments, R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51}.

In some embodiments, p is 0, 1, 2, 3, or 4.

In some embodiments, p is 0, 1, 2, or 3.

In some embodiments, p is 1, 2, 3, or 4.

In some embodiments, p is 1, 2, or 3.

In some embodiments, p is 0.

In some embodiments, p is 1.

In some embodiments, p is 2.

In some embodiments, p is 3.

In some embodiments, each R^{a51} is independently selected from halo, CN, and OR^{a52}.

In some embodiments, R^{b51} and R^{c51} are each independently selected from H and C₁₋₆ alkyl.

In some embodiments, R^{b51} and R^{c51} are each independently selected from H and C₁₋₃ alkyl.

In some embodiments, each R^{a52} is selected from H and C₁₋₆ alkyl.

In some embodiments, each R^{a52} is selected from H and C₁₋₃ alkyl.

In some embodiments, R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51};
p is 0, 1, 2, 3, or 4;
each R^{a51} is independently selected from halo, CN, and OR^{a52};
R^{b51} and R^{c51} are each independently selected from H and C₁₋₆ alkyl; and
R^{a52} is selected from H and C₁₋₆ alkyl.

In some embodiments, R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51};
p is 0, 1, 2, 3, or 4;
each R^{a51} is independently selected from halo, CN and OR^{a52};
R^{b51} and R^{c51} are each independently selected from H and C₁₋₆ alkyl; and
R^{a52} is selected from H and C₁₋₃ alkyl.

In some embodiments, R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51};
p is 0, 1, 2, 3, or 4;
each R^{a51} is independently selected from halo, CN and OR^{a52};
R^{b51} and R^{c51} are each independently selected from H and C₁₋₆ alkyl; and
R^{a52} is selected from H and methyl.

In some embodiments, R⁵ is selected from methyl, ethyl, isopropyl, difluoromethyl, trifluoromethyl, (trifluoromethyl)ethyl, fluoro, chloro, bromo, iodo, cyano, nitro, S(O)CH₃, S(O)₂CH₃, S(O)(NH)CH₃, C(O)N(CH₃)(CH(CH₃)₂), and —NHS(O)₂CH₃, wherein the methyl, ethyl, isopropyl, and (trifluoromethyl)ethyl of R⁵ are each optionally substituted with hydroxy, methoxy, or cyano.

In some embodiments, R⁵ is selected from methyl, difluoromethyl, trifluoromethyl, (trifluoromethyl)ethyl, (trifluoromethyl)(hydroxy)ethyl, hydroxymethyl, cyanomethyl, hydroxyethyl, hydroxyisopropyl, methoxyisopropyl, cyanoisopropyl, fluoro, chloro, bromo, iodo, cyano, nitro, S(O)CH₃, S(O)₂CH₃, S(O)(NH)CH₃, C(O)N(CH₃)(CH(CH₃)₂), and —NHS(O)₂CH₃.

In some embodiments:
R¹ is C₁₋₆ alkyl;
Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents;
each R² is independently selected from halo, oxo, C₁₋₆ alkyl, C₁₋₆ haloalkyl, C₂₋₆ alkenyl, and C₂₋₆ alkynyl;
R^{y2} is selected from H and C₁₋₆ alkyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents;
each R^{2A} is independently selected from C₁₋₆ alkyl, C₁₋₆ haloalkyl, C₂₋₆ alkenyl, C₂₋₆ alkynyl, and OR^{a21};
each R^{a21} is independently selected from H and C₁₋₆ alkyl;
R³ is selected from H and C₁₋₆ alkyl;
Cy⁴ is selected from 8-11 membered bicyclic heteroaryl, wherein the 8-11 membered bicyclic heteroaryl is optionally substituted with 1, 2, 3, 4, 5, 6, 7 or 8 independently selected R⁴ substituents;
each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4};
each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents;
each R^{4A} is independently selected from C₁₋₆ alkyl, C₁₋₆ haloalkyl, C₂₋₆ alkenyl, C₂₋₆ alkynyl, and OR^{a41};
each R^{a41} is independently selected from H and C₁₋₆ alkyl;
R⁵ is selected from —C₁₋₆ alkyl-(R^{a51}), halo, CN, NO₂, oxo, OR^{b51}, SR^{b51}, NR^{c51}R^{b51}, OC(O)R^{b51}, OC(NR^{c51})R^{b51}, OC(O)NR^{c51}R^{b51}, NR^{c51}C(O)R^{b51}, NR^{c51}C(O)OR^{b51}, NR^{c51}C(O)NR^{c51}R^{b51}, NR^{c51}SO₂R^{b51}, NR^{c51}SO₂NR^{c51}R^{b51}, NR^{c51}S(O)R^{b51}, C(O)OR^{b51}, C(O)R^{b51}, C(O)NR^{c51}R^{b51}, S(O)R^{b51}, S(O)NR^{c51}R^{b51}, S(O)₂R^{b51}, S(O)(NH)R^{b51}, and SO₂NR^{c51}R^{b51};
p is 0, 1, 2, 3, or 4;
each R^{a51} is independently selected from halo, CN, NO₂, oxo, OR^{a52}, NHR^{a52}, NR^{b52}R^{a52}, SR^{a52}, SO₂R^{a52}, C(O)R^{a52}, C(O)OR^{a52}, C(O)NHR^{a52}, C(O)NR^{b52}R^{a52}, OC(O)R^{a52}, S(O)R^{a52}, S(O)NR^{b52}R^{a52}, S(O)₂R^{a52}, and SO₂NR^{b52}R^{a52};
each R^{b51} and R^{c51} are independently selected from H, C₁₋₆ alkyl, and C₁₋₆ haloalkyl; and
each R^{a52} and R^{b52} are independently selected from H and C₁₋₆ alkyl.

In some embodiments:
R¹ is C₁₋₆ alkyl;
Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents;
each R² is independently selected from halo, oxo, C₁₋₆ alkyl, C₁₋₆ haloalkyl, C₂₋₆ alkenyl, and C₂₋₆ alkynyl;
R^{y2} is selected from H and C₁₋₆ alkyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents;
each R^{2A} is independently selected from C₁₋₆ alkyl, C₁₋₆ haloalkyl, C₂₋₆ alkenyl, C₂₋₆ alkynyl, and OR^{a21};
each R^{a21} is independently selected from H and C₁₋₆ alkyl;
R³ is selected from H and C₁₋₆ alkyl;
Cy⁴ is a 9-10 membered bicyclic heteroaryl, wherein the 9-10 membered bicyclic heteroaryl is optionally substituted with 1, 2, 3, or 4 independently selected R⁴ substituents;
each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4};
each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents;
each R^{4A} is independently selected from C₁₋₆ alkyl, C₁₋₆ haloalkyl, C₂₋₆ alkenyl, C₂₋₆ alkynyl, and OR^{a41};
each R^{a41} is independently selected from H and C₁₋₆ alkyl;
R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51};
p is 0, 1, 2, 3, or 4;
each R^{a51} is independently selected from halo, CN, NO₂, oxo, OR^{a52}, NHR^{a52}, NR^{b52}R^{a52}, SR^{a52}, SO₂R^{a52}, C(O)R^{a52}, C(O)OR^{a52}, C(O)NHR^{a52}, C(O)NR^{b52}R^{a52}, OC(O)R^{a52}, S(O)R^{a52}, S(O)NR^{b52}R^{a52}, S(O)₂R^{a52}, and SO₂NR^{b52}R^{a52};
each R^{b51} and R^{c51} are independently selected from H, C₁₋₆ alkyl, and C₁₋₆ haloalkyl; and
each R^{a52} and R^{b52} are independently selected from H and C₁₋₆ alkyl.

In some embodiments:
R¹ is C₁₋₆ alkyl;
Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents;
each R² is independently selected from halo, oxo, C₁₋₆ alkyl, C₁₋₆ haloalkyl, C₂₋₆ alkenyl, and C₂₋₆ alkynyl;
R^{y2} is selected from H and C₁₋₆ alkyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents;
each R^{2A} is independently selected from C₁₋₆ alkyl, C₁₋₆ haloalkyl, C₂₋₆ alkenyl, C₂₋₆ alkynyl, and OR^{a21};
each R^{a21} is independently selected from H and C₁₋₆ alkyl;
R³ is selected from H and C₁₋₆ alkyl;
Cy⁴ is selected from quinolinyl, naphthyridinyl, quinoxalinyl, and imidazo[1,2-b]pyridazinyl, wherein the quinolinyl, naphthyridinyl, quinoxalinyl, and imidazo[1,2-b]pyridazinyl are each optionally substituted with 1, 2, 3, or 4 independently selected R⁴ substituents;
each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4};
each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents;
each R^{4A} is independently selected from C₁₋₆ alkyl, C₁₋₆ haloalkyl, C₂₋₆ alkenyl, C₂₋₆ alkynyl, and OR^{a41};
each R^{a41} is independently selected from H and C₁₋₆ alkyl;
R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51};
p is 0, 1, 2, 3, or 4;
each R^{a51} is independently selected from halo, CN, NO₂, oxo, OR^{a52}, NHR^{a52}, NR^{b52}R^{a52}, SR^{a52}, SO₂R^{a52}, C(O)R^{a52}, C(O)OR^{a52}, C(O)NHR^{a52}, C(O)NR^{b52}R^{a52}, OC(O)R^{a52}, S(O)R^{a52}, S(O)NR^{b52}R^{a52}, S(O)₂R^{a52}, and SO₂NR^{b52}R^{a52};
each R^{b51} and R^{c51} are independently selected from H, C₁₋₆ alkyl, and C₁₋₆ haloalkyl; and
each R^{a52} and R^{b52} are independently selected from H and C₁₋₆ alkyl.

In some embodiments:
R¹ is C₁₋₆ alkyl;
Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents;
each R² is independently selected from halo, oxo, C₁₋₆ alkyl, C₁₋₆ haloalkyl, C₂₋₆ alkenyl, and C₂₋₆ alkynyl;
R^{y2} is selected from H and C₁₋₆ alkyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents;
each R^{2A} is independently selected from C₁₋₆ alkyl, C₁₋₆ haloalkyl, C₂₋₆ alkenyl, C₂₋₆ alkynyl, and OR^{a21};
each R^{a21} is independently selected from H and C₁₋₆ alkyl;
R³ is selected from H and C₁₋₆ alkyl;
Cy⁴ is selected from quinolinyl, naphthyridinyl, quinoxalinyl, and imidazo[1,2-b]pyridazinyl, wherein the quinolinyl, naphthyridinyl, quinoxalinyl, and imidazo[1,2-b]pyridazinyl are each optionally substituted with 1, 2, 3, or 4 independently selected R⁴ substituents;
each R⁴ is independently selected from methyl, isopropyl, trifluoromethyl, and NHC(O)R^{b4}, wherein each methyl and isopropyl of R⁴ is optionally substituted with 1 or 2 independently selected R^{4A} substituents;
each R^{b4} is independently selected from H and C₁₋₆ alkyl, wherein each C₁₋₆ alkyl of R^{b4} is optionally substituted with 1 or 2 independently selected R^{4A} substituents;
each R^{4A} is independently selected from C₁₋₆ alkyl, C₁₋₆ haloalkyl, C₂₋₆ alkenyl, C₂₋₆ alkynyl, and OR^{a41};
each R^{a41} is independently selected from H and C₁₋₆ alkyl;
R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51};
p is 0, 1, 2, 3, or 4;
each R^{a51} is independently selected from halo, CN, NO₂, oxo, OR^{a52}, NHR^{a52}, NR^{b52}R^{a52}, SR^{a52}, SO₂R^{a52}, C(O)R^{a52}, C(O)OR^{a52}, C(O)NHR^{a52}, C(O)NR^{b52}R^{a52}, OC(O)R^{a52}, S(O)R^{a52}, S(O)NR^{b52}R^{a52}, S(O)₂R^{a52}, and SO₂NR^{b52}R^{a52};
each R^{b51} and R^{c51} are independently selected from H, C₁₋₆ alkyl, and C₁₋₆ haloalkyl; and
each R^{a52} and R^{b52} are independently selected from H and C₁₋₆ alkyl.

In some embodiments:
R¹ is C₁₋₆ alkyl;
Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents;
each R² is independently selected from halo, oxo, C₁₋₆ alkyl, C₁₋₆ haloalkyl, C₂₋₆ alkenyl, and C₂₋₆ alkynyl;
R^{y2} is selected from H and C₁₋₆ alkyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents;
each R^{2A} is independently selected from C₁₋₆ alkyl, C₁₋₆ haloalkyl, C₂₋₆ alkenyl, C₂₋₆ alkynyl, and OR^{a21};
each R^{a21} is independently selected from H and C₁₋₆ alkyl;
R³ is selected from H and C₁₋₆ alkyl;
Cy⁴ is selected from quinolinyl, naphthyridinyl, quinoxalinyl, and imidazo[1,2-b]pyridazinyl, wherein the quinolinyl, naphthyridinyl, quinoxalinyl, and imidazo[1,2-b]pyridazinyl are each optionally substituted with 1, 2, 3, or 4 independently selected R⁴ substituents;
each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4};
each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents;
each R^{4A} is independently selected from C₁₋₆ alkyl, C₁₋₆ haloalkyl, C₂₋₆ alkenyl, C₂₋₆ alkynyl, and OR^{a41};
each R^{a41} is independently selected from H and C₁₋₆ alkyl; and
R⁵ is selected from methyl, difluoromethyl, trifluoromethyl, (trifluoromethyl)ethyl, (trifluoromethyl)(hydroxy)ethyl, hydroxymethyl, cyanomethyl, hydroxyethyl, hydroxyisopropyl, methoxyisopropyl, cyanoisopropyl, fluoro, chloro, bromo, iodo, cyano, nitro, S(O)CH₃, S(O)₂CH₃, S(O)(NH)CH₃, C(O)N(CH₃)(CH(CH₃)₂), and —NHS(O)₂CH₃.

In some embodiments:
R¹ is C₁₋₆ alkyl;
Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents;
each R² is independently selected from halo, oxo, C₁₋₆ alkyl, C₁₋₆ haloalkyl, C₂₋₆ alkenyl, and C₂₋₆ alkynyl;
R^{y2} is selected from H and C₁₋₆ alkyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents;
each R^{2A} is independently selected from C₁₋₆ alkyl, C₁₋₆ haloalkyl, C₂₋₆ alkenyl, C₂₋₆ alkynyl, and OR^{a21};
each R^{a21} is independently selected from H and C₁₋₆ alkyl;
R³ is selected from H and C₁₋₆ alkyl;
Cy⁴ is selected from quinolinyl, naphthyridinyl, quinoxalinyl, and imidazo[1,2-b]pyridazinyl, wherein the quinolinyl, naphthyridinyl, quinoxalinyl, and imidazo[1,2-b]pyridazinyl are each optionally substituted with 1, 2, 3, or 4 independently selected R⁴ substituents;
each R⁴ is independently selected from methyl, isopropyl, trifluoromethyl, and NHC(O)R^{b4}, wherein each methyl and isopropyl of R⁴ is optionally substituted with 1 or 2 independently selected R^{4A} substituents;
each R^{b4} is independently selected from H and C₁₋₆ alkyl, wherein each C₁₋₆ alkyl of R^{b4} is optionally substituted with 1 or 2 independently selected R^{4A} substituents;
each R^{4A} is independently selected from C₁₋₆ alkyl, C₁₋₆ haloalkyl, C₂₋₆ alkenyl, C₂₋₆ alkynyl, and OR^{a41};
each R^{a41} is independently selected from H and C₁₋₆ alkyl; and
R⁵ is selected from methyl, difluoromethyl, trifluoromethyl, (trifluoromethyl)ethyl, (trifluoromethyl)(hydroxy)ethyl, hydroxymethyl, cyanomethyl, hydroxyethyl, hydroxyisopropyl, methoxyisopropyl, cyanoisopropyl, fluoro, chloro, bromo, iodo, cyano, nitro, S(O)CH₃, S(O)₂CH₃, S(O)(NH)CH₃, C(O)N(CH₃)(CH(CH₃)₂), and —NHS(O)₂CH₃.

In some embodiments, the compound of Formula I is a compound of Formula II:

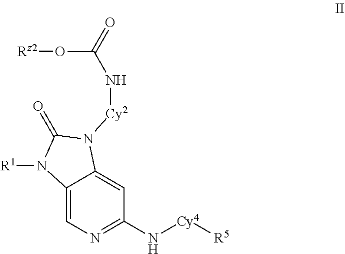

or a pharmaceutically acceptable salt thereof.

In some embodiments, the compound of Formula I or Formula II is a compound of Formula IIa, IIb, IIc, IId, IIe, IIf, IIg, IIh, IIi, or IIj:

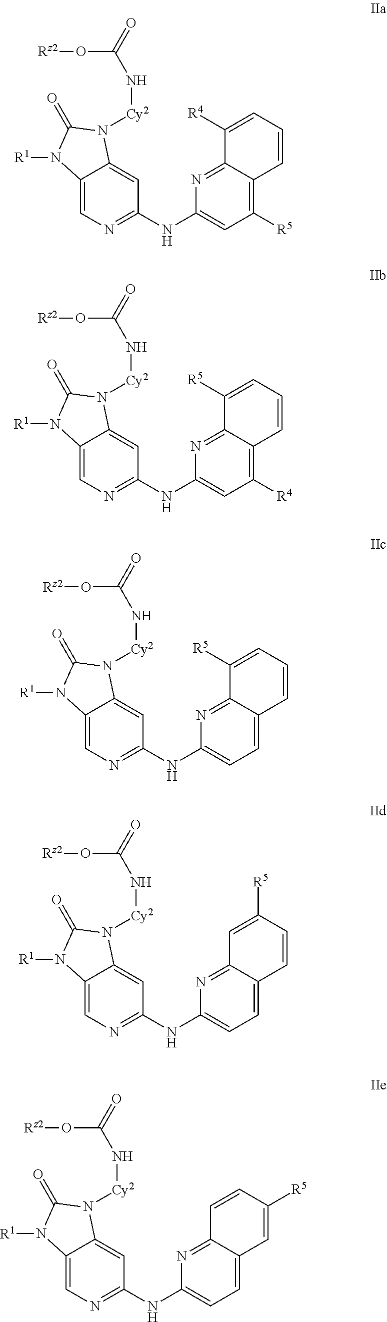

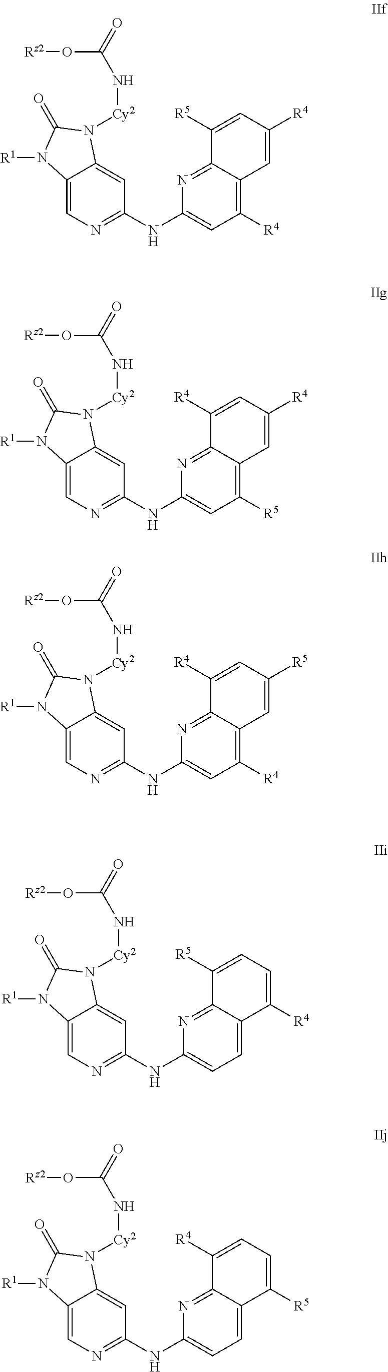

or a pharmaceutically acceptable salt thereof.

In some embodiments, the compound of Formula I is a compound of Formula III:

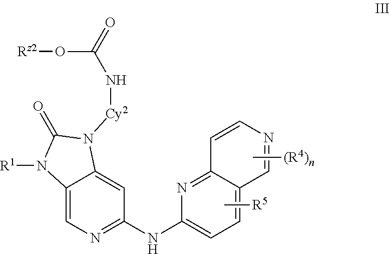

or a pharmaceutically acceptable salt thereof, wherein n is 0, 1, 2, 3, or 4.

In some embodiments, the compound of Formula I or Formula III is a compound of Formula IIIa:

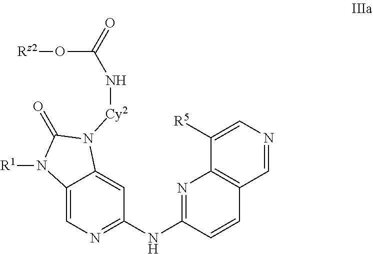

or a pharmaceutically acceptable salt thereof.

In some embodiments, the compound of Formula I is a compound of Formula IV:

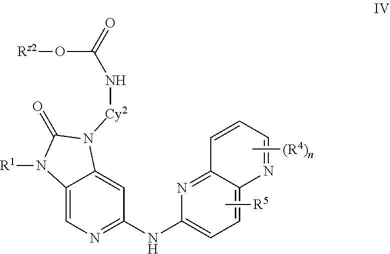

or a pharmaceutically acceptable salt thereof, wherein n is 0, 1, 2, 3, or 4.

In some embodiments, the compound of Formula I or Formula IV is a compound of Formula IVa, IVb, or IVc:

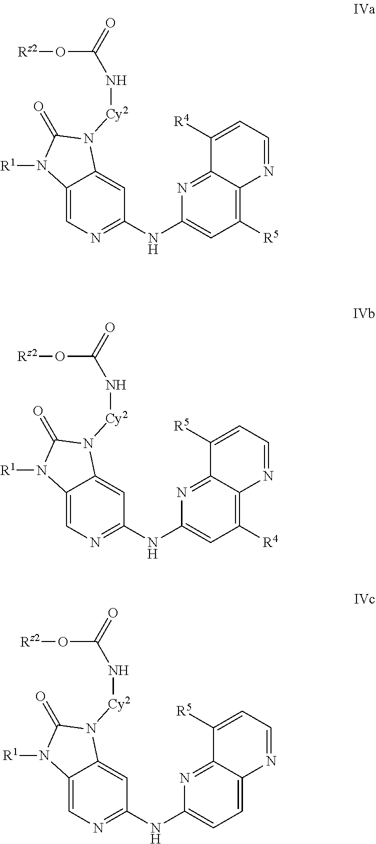

or a pharmaceutically acceptable salt thereof.

In some embodiments, the compound of Formula I is a compound of Formula V:

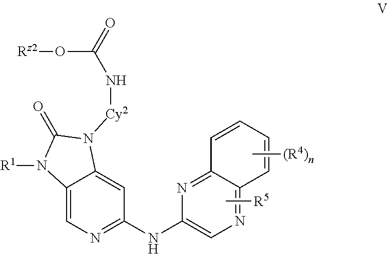

or a pharmaceutically acceptable salt thereof, wherein n is 0, 1, 2, 3, or 4.

In some embodiments, the compound of Formula I or Formula V is a compound of Formula Va:

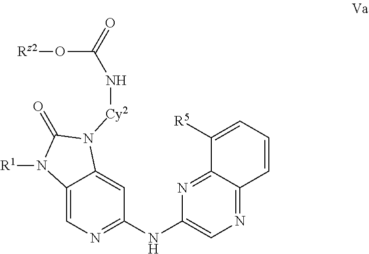

or a pharmaceutically acceptable salt thereof.

In some embodiments, the compound of Formula I is a compound of Formula VI:

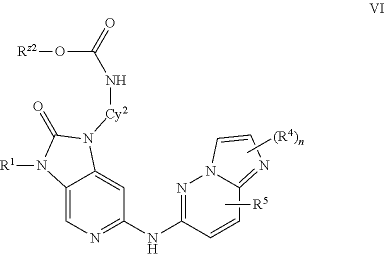

or a pharmaceutically acceptable salt thereof, wherein n is 0, 1, 2, or 3.

In some embodiments, the compound of Formula I or Formula VI is a compound of Formula VIa, Vib, Vic, or VId:

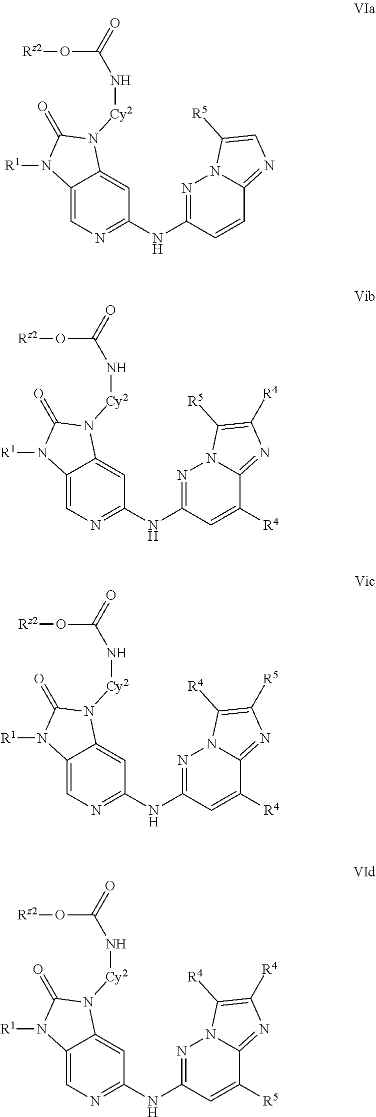

or a pharmaceutically acceptable salt thereof.

In some embodiments, the compound provided herein is selected from:
methyl ((1R,3R)-3-(6-((4-(hydroxymethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(1-hydroxyethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-2-oxo-6-((4-(1,1,1-trifluoro-2-hydroxypropan-2-yl)-8-(trifluoromethyl)quinolin-2-yl)amino)-2,3-dihydro-1H-imidazo[4,5-c]pyridin-1-yl)cyclopentyl)carbamate;
methyl ((1R,3R)-3-(3-(methyl-d₃)-6-((4-methyl-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(3-(methyl-d₃)-6-((4-(methylsulfinyl)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(3-(methyl-d₃)-6-((4-(methylsulfonyl)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(3-(methyl-d₃)-6-((4-(S-methylsulfonimidoyl)-8-(trifluoromethyl)quinolin-2-yl)amino)-2-oxo-2,3-dihydro-1H-imidazo[4,5-c]pyridin-1-yl)cyclopentyl)carbamate;
benzyl ((1R,3R)-3-(3-(methyl-d₃)-2-oxo-6-((8-(trifluoromethyl)quinolin-2-yl)amino)-2,3-dihydro-1H-imidazo[4,5-c]pyridin-1-yl)cyclopentyl)carbamate;
methyl ((1R,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(isopropyl(methyl)carbamoyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate;
2-methoxyethyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate;
methyl ((trans)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclobutyl)carbamate;
cyclopropyl ((trans)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclobutyl)carbamate;
methyl ((1R,3R)-3-(6-((4-(2-methoxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(cyanomethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-cyanopropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-chloro-4-(2-hydroxypropan-2-yl)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate;
methyl ((1R,3R)-3-(6-((4-chloro-8-(trifluoromethyl)quinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((4-(2-methoxyacetamido)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-6-((4-(methylsulfonamido)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(6-((8-bromoquinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-chloro-8-(trifluoromethyl)quinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-cyano-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-fluoro-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-6-((8-methyl-1,6-naphthyridin-2-yl)amino)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-8-methyl-1,5-naphthyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-2-oxo-6-((3-(1,1,1-trifluoropropan-2-yl)imidazo[1,2-b]pyridazin-6-yl)amino)-2,3-dihydro-1H-imidazo[4,5-c]pyridin-1-yl)cyclopentyl)carbamate;
methyl ((1R,3R)-3-(3-(methyl-d₃)-2-oxo-6-((3-(trifluoromethyl)imidazo[1,2-b]pyridazin-6-yl)amino)-2,3-dihydro-1H-imidazo[4,5-c]pyridin-1-yl)cyclopentyl)carbamate;
methyl (4-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)bicyclo[2.1.1]hexan-1-yl)carbamate;
methyl (3-(3-(methyl-d₃)-2-oxo-6-((8-(trifluoromethyl)quinolin-2-yl)amino)-2,3-dihydro-1H-imidazo[4,5-c]pyridin-1-yl)bicyclo[1.1.1]pentan-1-yl)carbamate;
methyl ((1R,3R)-3-(6-((8-(2-cyanopropan-2-yl)-4-(2-hydroxypropan-2-yl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-6-((8-nitroquinolin-2-yl)amino)-2-oxo-2,3-dihydro-1H-imidazo[4,5-c]pyridin-1-yl)cyclopentyl)carbamate;
methyl ((1R,3R)-3-(6-((8-cyano-1,5-naphthyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((8-cyanoquinoxalin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((8-(difluoromethyl)quinolin-2-yl)amino)-3-methyl-2-oxo-2,3-dihydro-1H-imidazo[4,5-c]pyridin-1-yl)cyclopentyl)carbamate;
methyl ((1R,3R)-3-(3-methyl-6-((8-methylquinolin-2-yl)amino)-2-oxo-2,3-dihydro-1H-imidazo[4,5-c]pyridin-1-yl)cyclopentyl)carbamate;
methyl ((1S,3S)-3-(6-((8-chloroquinolin-2-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((8-(trifluoromethyl)quinolin-2-yl)amino)-2,3-dihydro-1H-imidazo[4,5-c]pyridin-1-yl)cyclopentyl)carbamate;
methyl ((1R,3R)-3-(6-((3-cyanoimidazo[1,2-b]pyridazin-6-yl)amino)-3-methyl-2-oxo-2,3-dihydro-1H-imidazo[4,5-c]pyridin-1-yl)cyclopentyl)carbamate;
methyl ((1R,3R)-3-(6-((8-cyanoquinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((7-cyanoquinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-cyanoquinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((3-iodo-2,8-dimethylimidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate; and
methyl ((1R,3R)-3-(6-((5-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
   or a pharmaceutically acceptable salt thereof

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The term “Cₙ₋ₘ alkyl” is understood to include deuterated analogs of saturated hydrocarbon groups as defined herein, including but not limited to, groups such as trideuteromethyl (CD₃), pentadeuteroethyl (CD₂CD₃), and the like.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms. The term “Cₙ₋ₘ alkenyl” is understood to include deuterated analogs of alkenyl groups as defined herein, including but not limited to, groups such as trideuteroethenyl (—CD=CD₂), tetradeuteropropenyl, (—CD=CD-CD₂), and the like.

As used herein, “Cₙ₋ₘ alkynyl” refers to an alkyl group having one or more triple carbon-carbon bonds and having n to m carbons. Example alkynyl groups include, but are not limited to, ethynyl, propynyl (e.g., propyn-1-yl, propyn-2-yl, prop-2-yn-1-yl), and the like. In some embodiments, the alkynyl moiety contains 2 to 6, 2 to 4, or 2 to 3 carbon atoms. The term “Cₙ₋ₘ alkynyl” is understood to include deuterated analogs of alkynyl groups as defined herein, including but not limited to, groups such as deuteroethynyl (—C≡CD), trideuteropropyn-1-yl, (—C≡CCD₃), and the like.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. The term “Cₙ₋ₘ alkoxy” is understood to include deuterated analogs of the alkyl moiety of the alkoxy groups as defined herein, including but not limited to, groups such as trideuteromethoxy (—OCD₃), pentadeuteroethoxy (—OCD₂CD₃), and the like.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl. The term “aryl” is understood to include deuterated analogs of the aryl groups as defined herein, including but not limited to, groups such as pentadeuterophenyl (i.e., perdeuterophenyl, phenyl-d₅), perdeuteronaphthyl, and the like.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like. The term “Cₙ₋ₘ haloalkyl” is understood to include deuterated analogs of the haloalkyl groups as defined herein, including but not limited to, groups such as deuterodifluoromethyl (—CDF₂), dideuterofluoromethyl (—CD₂F), and the like.

As used herein, the term “carbonyl”, employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, azaspiro[2.4]heptanyl, and the like. In some embodiments, cycloalkyl is cyclopropyl, cyclobutyl, cyclopentyl, or cyclohexyl. The term “cycloalkyl” is understood to include deuterated analogs of the cycloalkyl groups as defined herein, including but not limited to, groups such as perdeuterocyclopropyl, perdeuterocyclobutyl, perdeuterocyclopentyl, perdeuterocyclohexyl, and the like.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1, 2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, pyrazolo[1,5-a]pyrimidinyl, indazolyl, imidazo[1,2-b]pyridazinyl, pyrazolo[1,5-a]pyrimidinyl, and the like. The term “heteroaryl” is understood to include deuterated analogs of the heteroaryl groups as defined herein, including but not limited to, groups such as perdeuteropyridinyl, perdeuteropyrazinyl, perdeuteropyrimidinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. The term “heterocycloalkyl” is understood to include deuterated analogs of the heterocycloalkyl groups as defined herein, including but not limited to, groups such as perdeuteroazetidinyl, perdeuteropyrrolidinyl, perdeuteropiperidinyl, and the like.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 5,6-dihydro-4H-pyrrolo[1,2-b]pyrazolyl, 3-oxopiperazinyl, oxo-pyrrolidinyl, oxo-pyridinyl, 2,3-dihydrobenzo[b][1,4]dioxinyl, 3,4-dihydro-2H-benzo[b][1,4]oxazinyl, octahydro-2H-pyrido[1,2-a]pyrazinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms. The term “Cₚ cycloalkyl-Cₙ₋ₘ alkyl-” is understood to include deuterated analogs of the cycloalkyl and/or alkyl moieties of the Cₒ₋ₚ cycloalkyl-Cₙ₋ₘ alkyl- groups as defined herein.

As used herein “Cₚ aryl-Cₙ₋ₘ alkyl-” refers to a group of formula aryl-alkylene-, wherein the aryl has o to p carbon atoms and the alkylene linking group has n to m carbon atoms. The term “Cₒ₋ₚ aryl-Cₙ₋ₘ alkyl-” is understood to include deuterated analogs of the aryl and/or alkyl moieties of the Cₒ₋ₚ aryl-Cₙ₋ₘ alkyl- groups as defined herein.

As used herein, “heteroaryl-Cₙ₋ₘ alkyl-” refers to a group of formula heteroaryl-alkylene-, wherein alkylene linking group has n to m carbon atoms. The term “heteroaryl-Cₙ₋ₘ alkyl-” is understood to include deuterated analogs of the heteroaryl and/or alkyl moieties of the heteroaryl-Cₙ₋ₘ alkyl- groups as defined herein.

As used herein “heterocycloalkyl-Cₙ₋ₘ alkyl-” refers to a group of formula heterocycloalkyl-alkylene-, wherein alkylene linking group has n to m carbon atoms. The term “heterocycloalkyl-Cₙ₋ₘ alkyl-” is understood to include deuterated analogs of the heterocycloalkyl and/or alkyl moieties of the heterocycloalkyl-Cₙ₋ₘ alkyl- groups as defined herein.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like. The terms “alkyl linking group” and “alkylene linking group” are understood to include deuterated analogs of the alkylene groups as defined herein.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula I can be prepared, for example, using a process as illustrated in Scheme 1. In the process depicted in Scheme 1, the compounds of formula 1-1, where L₁ is a halogen (e.g., F, Cl, or Br), can react with the compounds 1-2 via nucleophilic aromatic substitution reactions (e.g., in the presence of a base, such as N,N-diisopropylethylamine) followed by reduction of the nitro group in compound 1-3 (e.g. under reductive conditions, such as treatment with Zn powder and NH₄Cl) resulting in the formation of the compounds of formula 1-4. The compounds of formula 1-4 can be converted to cyclic urea 1-5 under standard conditions (e.g., in the presence of N-succinimidyl carbonate). The compounds of formula 1-5 can be further transformed to the compounds of formula 1-6 via C—N bond formation (e.g., in the presence of alkyl halide and a base). The compounds of formula 1-6, where L₂ is a halogen (e.g., Cl, or Br), can react with the compounds of formula 1-7 via Buchwald-Hartwig cross-coupling reactions (e.g., in the presence of tris(dibenzylideneacetone)dipalladium(0), Xantphos and cesium carbonate) to yield the compounds of formula 1-8. The compounds of formula 1-8 can undergo NH deprotection (e.g., treatment with HCl or TFA), followed by carbamate formation (e.g., in the presence of chloroformate and a base) to form the compounds of Formula I.

Alternatively, the compounds of formula 1-6 can be used to prepare compounds of formula 1-9 through NH deprotection (e.g., treatment with HCl or TFA), followed by carbamate formation (e.g., in the presence of chloroformate and a base). Then, the compounds of formula 1-9 can be coupled with the compounds of formula 1-7 under Buchwald-Hartwig cross-coupling reactions (e.g., in the presence of tris(dibenzylideneacetone)dipalladium(0), Xantphos and cesium carbonate) to afford the compounds of Formula I.

The compounds of formula 1-9 can be used to prepare compounds of formula 1-10 through Buchwald-Hartwig cross-coupling reactions (e.g., in the presence of tris(dibenzylideneacetone)dipalladium(0), Xantphos and cesium carbonate) followed by NH deprotection of the protecting group (e.g., treatment with HCl or TFA). Then, the compounds of formula 1-10 can be coupled with the compounds of formula 1-11, where X is a halogen (e.g., Cl, or Br), under Buchwald-Hartwig cross-coupling reactions (e.g., in the presence of tris(dibenzylideneacetone)dipalladium(0), Xantphos and cesium carbonate) to generate the compounds of Formula I.

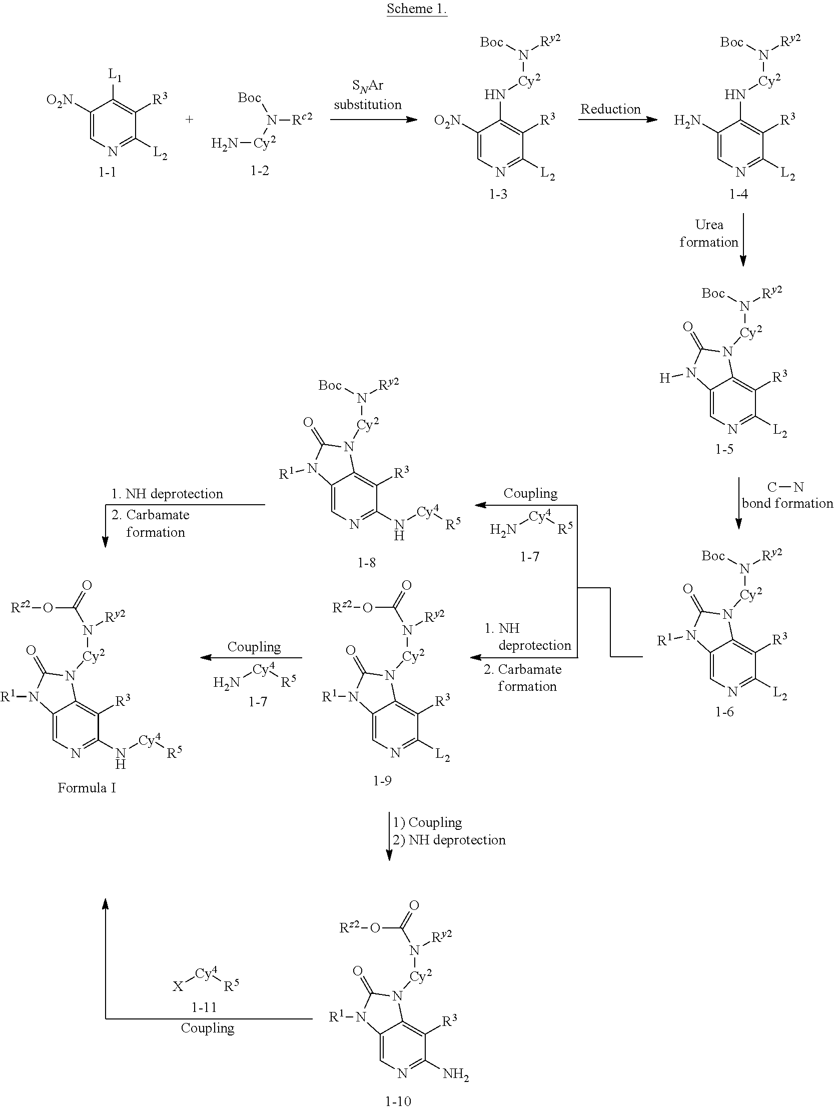

When Cy⁴ is a 10-membered bicyclic heteroaryl ring, the compounds of formula 1-7 and 1-11 can be prepared, for example, using processes as illustrated in Schemes 2-4.

In the process shown in Scheme 2, the compounds of formula 2-1 can undergo ring expansion under acidic condition (e.g., in the presence acetic acid), to give compounds of formula 2-2. Then, the compounds of formula 2-2 can be converted to compounds of formula 2-3 via esterification (e.g., in the presence of alcohol and SOCl₂) and chlorination (e.g., treatment with POCl₃). After functional group manipulations (e.g., amide coupling, reduction, hydrolysis, Grignard addition etc.) on the compound 2-3, the compounds of formula 1-11 can be prepared. The compounds of formula 1-11 can be elaborated into the compounds of formula 1-7 through Buchwald-Hartwig cross-coupling reactions (e.g., in the presence of a palladium catalyst and a suitable ligand) followed by NH deprotection of the protecting group (e.g., treatment with HCl or TFA).

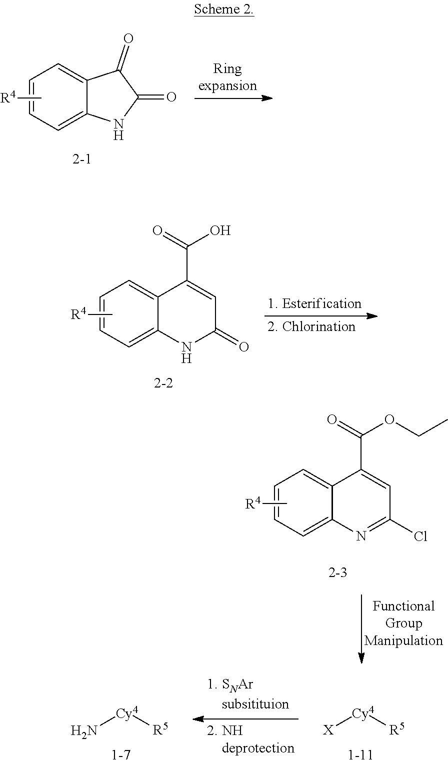

The compounds of formula 3-1 can undergo amide formation (e.g., in the presence of an acid chloride and a suitable base such as triethylamine) followed by cycloacylation reaction (e.g., in the presence of dehydrating reagents such as Eaton's reagent) to provide the compounds of formula 3-3. The compounds of formula 3-3 can be converted to compounds of formula 1-11 under chlorination reaction (e.g., treatment with POCl₃). The compounds of formula 1-11 can be elaborated into the compounds of formula 1-7 through Buchwald-Hartwig cross-coupling reactions (e.g., in the presence of a palladium catalyst and a suitable ligand) followed by NH deprotection of the protecting group (e.g., treatment with HCl or TFA) as depicted in Scheme 3.

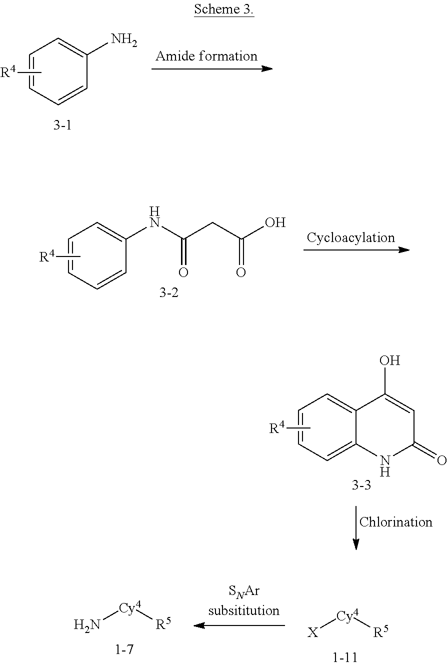

Alternatively, the compounds of formula 1-7 and 1-11 can be prepared, for example, using a process as illustrated in Scheme 4. The compounds of formula 4-1 can undergo coupling reaction with ethyl acrylate 4-2 under standard Heck reaction conditions (e.g., in the presence of a palladium catalyst and a suitable ligand/base) to generate the compounds of formula 4-3. The compounds of formula 4-3 can be subjected to cyclization reaction under acidic condition (e.g., in the presence of concentrated HCl) to provide the compounds of formula 4-4. Chlorination of the compound 4-4 (e.g., in the presence POCl₃) affords the compound of formula 1-11. The compounds of formula 1-11 can be elaborated into the compounds of formula 1-7 through Buchwald-Hartwig cross-coupling reactions (e.g., in the presence of a palladium catalyst and a suitable ligand) followed by NH deprotection of the protecting group (e.g., treatment with HCl or TFA).

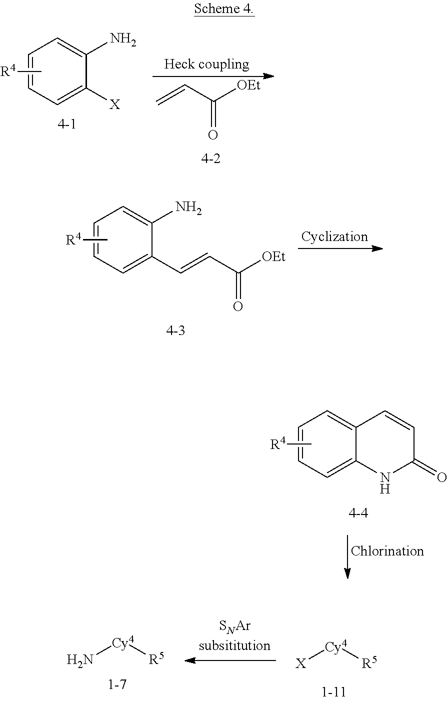

When Cy⁴ is imidazo[1,2-b]pyridazine ring, the compounds of formula 1-7 can be prepared, for example, using a process as illustrated in Scheme 5. The compounds of formula 5-1 can couple with the compounds 5-2 (e.g., M is B(OR)₂ Zn-hal, etc.) under standard Suzuki cross-coupling conditions (e.g., in the presence of a palladium catalyst and a suitable ligand/base), or standard Negishi cross-coupling conditions (e.g., in the presence of a palladium catalyst and a suitable ligand), to generate the compound of formula 5-3. Iodination of the compound 5-3 (e.g., in the presence of N-iodosuccinimide) affords the compound of formula 5-4. The compounds of formula 5-4 can react with the compounds 5-5 (e.g., M is B(OR)₂) under standard cross-coupling conditions (e.g., in the presence of a palladium catalyst and a suitable ligand/base) to give compounds of formula 5-6 (1-11). The compounds of formula 5-6 can be elaborated into the compounds of formula 1-7 through Buchwald-Hartwig cross-coupling reactions (e.g., in the presence of a palladium catalyst and a suitable ligand) followed by NH deprotection of the protecting group (e.g., treatment with HCl or TFA).

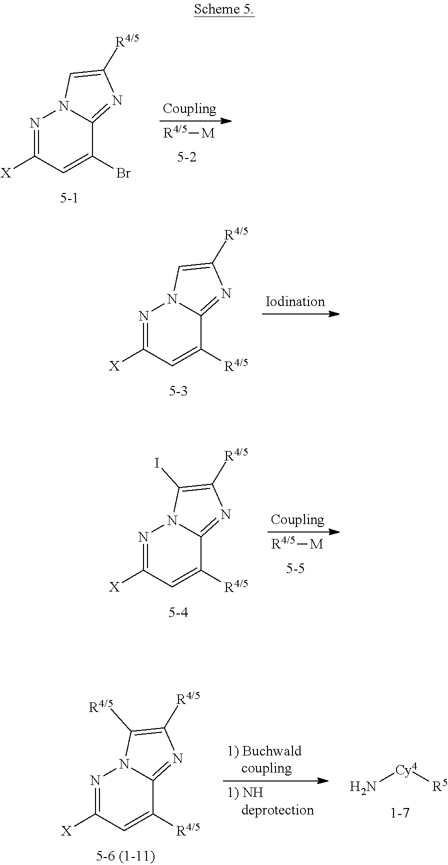

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “t” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can inhibit the activity of the V617F variant of the protein-tyrosine kinase JAK2 (i.e., “V617F” or “JAK2V617F”). Compounds which inhibit V617F are useful in providing a means of preventing the growth or inducing apoptosis in tumors, particularly by inhibiting angiogenesis. It is therefore anticipated that the compounds of the disclosure are useful in treating or preventing proliferative disorders such as cancers. In particular tumors with activating mutants of receptor tyrosine kinases or upregulation of receptor tyrosine kinases may be particularly sensitive to the inhibitors.

In certain embodiments, the disclosure provides a method for treating a V617F-related disorder in a patient in need thereof, comprising the step of administering to said patient a compound of the disclosure, or a pharmaceutically acceptable composition thereof.

Myeloproliferative diseases (MPD) are multipotent hematopoietic stem cell disorders characterized by excess production of various blood cells. MPNs include polycythemia vera (PV), essential thrombocythemia (ET), and idiopathic myelofibrosis (IMF). JAK2 V617F mutation is reported in about 95% of patients with PV, in 35% to 70% of patients with ET, and 50% of patients with IMF. Also, JAK2 exon 12 mutations are detected in some of the V617F-negative PV patients (Ma et al., J. Mol. Diagn., 11: 49-53, 2009). In some embodiments, the compounds of the disclosure can be useful in the treatment of myeloproliferative disorders (e.g., myeloproliferative neoplasms) in a patient in need thereof, such as polycythemia vera, essential thrombocythemia, myelofibrosis with myeloid metaplasia (MMM), primary myelofibrosis (PMF), chronic myelogenous leukemia (CML), chronic myelomonocytic leukemia (CMML), hypereosinophilic syndrome (HES), systemic mast cell disease (SMCD), and the like.

In some embodiments, the myeloproliferative disorder is selected from polycythemia vera, essential thrombocythemia, myelofibrosis with myeloid metaplasia, primary myelofibrosis, post-essential thrombocythemia myelofibrosis, and post polycythemia vera myelofibrosis.

In some embodiments, the myeloproliferative disorder is a myeloproliferative neoplasm.

In some embodiments, the myeloproliferative disorder is myelofibrosis (e.g., primary myelofibrosis (PMF) or post polycythemia vera/essential thrombocythemia myelofibrosis (Post-PV/ET MF)).

In some embodiments, the myeloproliferative disorder is primary myelofibrosis (PMF).

In some embodiments, the myeloproliferative disorder is post-essential thrombocythemia myelofibrosis (Post-ET MF).

In some embodiments, the myeloproliferative disorder is post polycythemia vera myelofibrosis (Post-PV MF).

In some embodiments, the myeloproliferative disorder is selected from primary myelofibrosis (PMF), polycythemia vera (PV), and essential thrombocythemia (ET).

In some embodiments, the myeloproliferative neoplasm is primary myelofibrosis (PMF).

In some embodiments, the myeloproliferative neoplasm is polycythemia vera (PV).

In some embodiments, the myeloproliferative neoplasm is essential thrombocythemia (ET).

Myeloproliferative diseases include disorders of a bone marrow or lymph node-derived cell type, such as a white blood cell. A myeloproliferative disease can manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CML), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

For example, the compounds of the disclosure are useful in the treatment of cancer. Example cancers include bladder cancer (e.g., urothelial carcinoma, squamous cell carcinoma, adenocarcinoma), breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma (e.g., intrahepatic, hilar or perihilar, distal extrahepatic), liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g. exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

Further example cancers include hematopoietic malignancies such as leukemia or lymphoma, multiple myeloma, chronic lymphocytic lymphoma, adult T cell leukemia, acute myeloid leukemia (AML), B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera (PV), essential thrombocythemia (ET), and primary myelofibrosis (PMF)), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphomas, and Burkitt's lymphoma.

In certain embodiments, provided herein is a method of treating cancer comprising administering to a patient in need thereof a therapeutically effect amount of a compound of the disclosure. In certain embodiments, the cancer is selected from T lymphoblastic lymphoma, glioblastoma, melanoma, rhabdosarcoma, lymphosarcoma, and osteosarcoma.

Other cancers treatable with the compounds of the disclosure include tumors of the eye, glioblastoma, melanoma, leiomyosarcoma, and urothelial carcinoma (e.g., ureter, urethra, bladder, urachus).

The compounds of the disclosure can also be useful in the inhibition of tumor metastases.

In some embodiments, the compounds of the disclosure as described herein can be used to treat Alzheimer's disease, HIV, or tuberculosis.

In some embodiments, the compounds of the disclosure can be useful in the treatment of myelodysplastic syndrome (MDS) in a patient in need thereof. In some embodiments, said patient having the myelodysplastic syndrome (MDS) is red blood cell transfusion dependent.

As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥10% of 1 cell |
| unilineage dysplasia | | line, <5% blasts |
| (RCUD) | | |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × | Dysplasia in ≥10% of cells |
| multilineage dysplasia | 10⁹/L monocytes | in ≥2 hematopoietic |
| | | lineages, ±15% ring |
| | | sideroblasts, <5% blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% to |
| | monocytes | 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ±Auer rods, 10% to |
| | monocytes | 19% blasts |
| Myelodysplastic | Cytopenias | Unilineage or no dysplasia but |
| syndrome, unclassified | | characteristic MDS |
| (MDS-U) | | cytogenetics, <5% blasts |
| MDS associated with | Anemia, platelets | Unilineage erythroid. Isolated |
| isolated del(5q) | normal or increased | del(5q), <5% blasts |

In some embodiments, the myelodysplastic syndrome is refractory cytopenia with unilineage dysplasia (RCUID).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del(5q).

In some embodiments, the myelodysplastic syndrome is refractory to erythropoiesis-stimulating agents.

In some embodiments, the compounds of the disclosure can be useful in the treatment of myeloproliferative disorder/myelodysplastic overlap syndrome (MPD/MDS overlap syndrome).

In some embodiments, the compounds of the disclosure can be useful in the treatment of leukemia.

In some embodiments, the compounds of the disclosure can be useful in the treatment of acute myeloid leukemia (AML).

In addition to oncogenic neoplasms, the compounds of the disclosure can be useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes.

The compounds provided herein may further be useful in the treatment of fibrotic diseases, such as where a disease symptom or disorder is characterized by fibrosis. Example fibrotic diseases include liver cirrhosis, glomerulonephritis, pulmonary fibrosis, systemic fibrosis, rheumatoid arthritis, and wound healing.

In some embodiments, the compounds provided herein can be used in the treatment of a hypophosphatemia disorder such as, for example, X-linked hypophosphatemic rickets, autosomal recessive hypophosphatemic rickets, and autosomal dominant hypophosphatemic rickets, or tumor-induced osteromalacia.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a V617F variant with a compound described herein includes the administration of a compound described herein to an individual or patient, such as a human, having a V617F variant, as well as, for example, introducing a compound described herein into a sample containing a cellular or purified preparation containing the V617F variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein for treatment or prevention of V617F-associated diseases, disorders or conditions, or diseases or conditions as described herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFδR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein can be used in combination with one or more inhibitors of the enzyme or protein receptors such as HPK1, SBLB, TUT4, A2A/A2B, CD19, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds described herein can be used in combination with targeted therapies, including JAK kinase inhibitors (ruxolitinib, additional JAK1/2 and JAK1-selective, baricitinib or itacitinib), Pim kinase inhibitors (e.g., LGH447, INCB053914 and SGI-1776), PI3 kinase inhibitors including PI3K-delta selective and broad spectrum PI3K inhibitors (e.g., INCB50465 and INCB50797), PI3K-gamma inhibitors such as PI3K-gamma selective inhibitors, MEK inhibitors, CSF1R inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibitors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, compounds described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US 20170107216, US 20170145025, US 20170174671, US 20170174679, US 20170320875, US 20170342060, US 20170362253, and US 20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is retifanlimab (also known as MGA012), nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is retifanlimab. In some embodiments, the anti-PD1 antibody is SHR-1210. Other anti-cancer agent(s) include antibody therapeutics such as 4-1BB (e.g. urelumab, utomilumab.

In some embodiments, the compounds of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the compounds described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (IEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with compounds described herein for the treatment of cancer include chemotherapeutic agents, targeted cancer therapies, immunotherapies or radiation therapy. Compounds described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with inhibitors described herein. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib Activation of intracellular signaling pathways is frequent in cancer, and agents targeting components of these pathways have been combined with receptor targeting agents to enhance efficacy and reduce resistance. Examples of agents that may be combined with compounds described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds described herein. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein. In some embodiments, the JAK inhibitor is selective for JAK1 over JAK2 and JAK3.

Other suitable agents for use in combination with compounds described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (e.g., vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel, mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure can be used in combination with bone marrow transplant for the treatment of a variety of tumors of hematopoietic origin (see e.g., U.S. Pat. Nos. 9,233,985, 10,065,974, 10,287,303, 8,524,867, the disclosures of which are incorporated by reference herein in their entireties).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating V617F in tissue samples, including human, and for identifying V617F inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes V617F assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ (i.e., trideuteromethyl) being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

In some embodiments, the compound provided herein (e.g., the compound of any of Formulas I-VId), or a pharmaceutically acceptable salt thereof, comprises at least one deuterium atom.

In some embodiments, the compound provided herein (e.g., the compound of any of Formulas I-VId), or a pharmaceutically acceptable salt thereof, comprises two or more deuterium atoms.

In some embodiments, the compound provided herein (e.g., the compound of any of Formulas I-VId), or a pharmaceutically acceptable salt thereof, comprises three or more deuterium atoms.

In some embodiments, for a compound provided herein (e.g., the compound of any of Formulas I-VId), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro V617F labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind V617F by monitoring its concentration variation when contacting with V617F, through tracking of the labeling. For example, a test compound (labeled) can be evaluated for its ability to reduce binding of another compound which is known to bind to V617F (i.e., standard compound). Accordingly, the ability of a test compound to compete with the standard compound for binding to V617F directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of V617F-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)).

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 30×100 mm or Waters Xbridge™ Cl₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

pH=10 purifications: Waters Xbridge™ Cl₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

### Intermediate 1. Methyl ((1R,3R)-3-(6-chloro-3-(methyl-d₃)-2-oxo-2,3-dihydro-1H-imidazo[4,5-c]pyridin-1-yl)cyclopentyl)carbamate

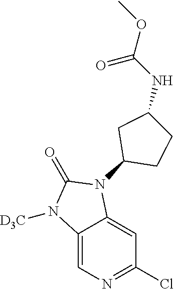 [SMILES: [2H]C([2H])([2H])n1c(=O)n([C@@H]2CC[C@@H](NC(=O)OC)C2)c2cc(Cl)ncc21]

### Step 1. Tert-Butyl ((1R,3R)-3-((2-chloro-5-nitropyridin-4-yl)amino)cyclopentyl)carbamate

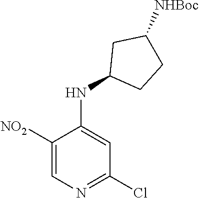 [SMILES: CC(C)(C)OC(=O)N[C@@H]1CC[C@@H](Nc2cc(Cl)ncc2[N+](=O)[O-])C1]

To a flask containing 2,4-dichloro-5-nitropyridine (18 g, 94 mmol) and DIPEA (19 mL, 108 mmol) in acetonitrile (45 mL) was added tert-butyl ((1R,3R)-3-aminocyclopentyl)carbamate (18 g, 90 mmol). The reaction mixture was heated to 55° C. for 2 h and then cooled to r.t. The mixture was diluted with water (150 mL) and filtered. The solid was washed with water (30 mL) for 3 times and dried in vacuo. The obtained crude product was used in the next step without further purification. LCMS calculated for C₁₅H₂₂ClN₄O₄(M+H)⁺: m/z=357.1; found 357.2.

### Step 2. Tert-Butyl ((1R,3R)-3-((5-amino-2-chloropyridin-4-yl)amino)cyclopentyl)carbamate

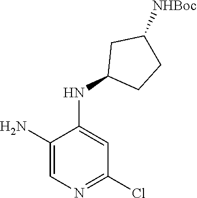 [SMILES: CC(C)(C)OC(=O)N[C@@H]1CC[C@@H](Nc2cc(Cl)ncc2N)C1]

A round-bottom flask containing zinc powder (57 g, 869 mmol) and ammonium chloride (47 g, 869 mmol) in MeOH (800 mL) was placed in a water bath at r.t. A solution of tert-butyl ((1R,3R)-3-((2-chloro-5-nitropyridin-4-yl)amino)cyclopentyl)carbamate (31 g, 86.9 mmol) in DCM/MeOH (200 mL, 20:1, v/v) was added via addition funnel to the zinc power suspension with vigorous stirring at the rate of 1 drop/sec. After stirring at r.t for 18 h, the reaction mixture was filtered and washed with DCM (200 mL) for three times. The filtrate was collected and concentrated. The residue was re-dissolved in DCM (300 mL), washed with water (100 mL) and extracted with DCM. The organic phase was washed with brine, dried over MgSO₄ and concentrated in vacuo. The obtained crude product was used in the next step without further purification. LCMS calculated for C₁₅H₂₄ClN₄O₂(M+H)⁺: m/z=327.2; found 327.2.

### Step 3. Tert-Butyl ((1R,3R)-3-(6-chloro-2-oxo-2,3-dihydro-1H-imidazo[4,5-c]pyridin-1-yl)cyclopentyl)carbamate

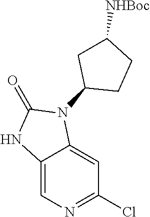 [SMILES: CC(C)(C)OC(=O)N[C@@H]1CC[C@@H](n2c(=O)[nH]c3cnc(Cl)cc32)C1]

To a flask containing tert-butyl ((1R,3R)-3-((5-amino-2-chloropyridin-4-yl)amino)cyclopentyl)carbamate (28 g, 86 mmol) in acetonitrile (280 mL) was added bis(2,5-dioxopyrrolidin-1-yl) carbonate (24 g, 94 mmol) in portions (over 15 times) during 30 min. After stirring at r.t for 16 h, the reaction mixture was filtered to obtain the solid product as off-white solid. The filtrate was concentrated in vacuo and washed with aqueous saturated sodium bicarbonate solution and extracted with DCM. The organic phase was washed with brine, dried over MgSO₄ and concentrated in vacuo. The obtained mixture was combined with the first portion of the product as off-white solid. The obtained crude product was used in the next step without further purification. LCMS calculated for C₁₆H₂₂ClN₄O₃(M+H)⁺: m/z=353.1; found: 353.1.

### Step 4. 1-((1R,3R)-3-Aminocyclopentyl)-6-chloro-3-(methyl-d₃)-1,3-dihydro-2H-imidazo[4,5-c]pyridin-2-one

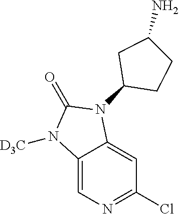 [SMILES: [2H]C([2H])([2H])n1c(=O)n([C@@H]2CC[C@@H](N)C2)c2cc(Cl)ncc21]

To a flask containing tert-butyl ((1R,3R)-3-(6-chloro-2-oxo-2,3-dihydro-1H-imidazo[4,5-c]pyridin-1-yl)cyclopentyl)carbamate (18.5 g, 52.4 mmol) and cesium carbonate (25.6 g, 79.0 mmol) in THF (131 mL) was added iodomethane-d₃ (15.2 g, 6.5 mL, 105 mmol). After stirring at r.t for 6 h, the reaction mixture was diluted with DCM (300 mL), filtered through a celite pad and washed with DCM (50 mL) for 3 times. The filtrate was washed with aqueous saturated sodium bicarbonate solution, and then brine, dried over MgSO₄ and concentrated in vacuo. The crude material was redissolved in DCM (20 ml) and TFA (50 ml). After stirring at 30° C. for 1 h, the reaction mixture was concentrated in vacuo. The crude material was redissolved in DCM (20 mL) and the pH of the mixture was adjusted to ˜10 with ammonia aqueous solution and then extracted into DCM. The organic phase was washed with brine, dried over MgSO₄ and concentrated in vacuo. The obtained crude product was used in the next step without further purification. LCMS calculated for C₁₂H₁₃D₃ClN₄O (M+H)⁺: m/z=270.1; found: 270.1.

### Step 5. Methyl ((1R,3R)-3-(6-chloro-3-(methyl-d₃)-2-oxo-2,3-dihydro-1H-imidazo[4,5-c]pyridin-1-yl)cyclopentyl)carbamate

To a flask containing 1-((1R,3R)-3-aminocyclopentyl)-6-chloro-3-(methyl-d₃)-1,3-dihydro-2H-imidazo[4,5-c]pyridin-2-one (13.5 g, 50 mmol) and DIPEA (7.8 g, 10.5 mL, 60.1 mmol) in DCM (330 mL) and MeOH (5 mL) at 0° C. was added methyl carbonochloridate (5.2 g, 4.3 mL, 55.1 mmol). After stirring at 0° C. for 5 min, the reaction mixture was quenched with MeOH and concentrated in vacuo. The obtained crude product was re-dissolved in DCM (250 mL) was washed with brine, dried over MgSO₄ and concentrated in vacuo. The product was purified by recrystallization from hot toluene to give the desired product as white solid. LCMS calculated for C₁₄H₁₅D₃ClN₄O₃ (M+H)⁺: m/z=328.1; found: 328.1.

### Intermediate 2. 2-(2-Amino-8-(trifluoromethyl)quinolin-4-yl)propan-2-ol

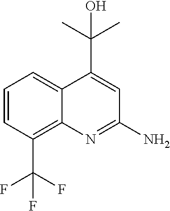 [SMILES: CC(C)(O)c1cc(N)nc2c(C(F)(F)F)cccc12]

### Step 1. 2-Oxo-8-(trifluoromethyl)-1,2-dihydroquinoline-4-carboxylic acid

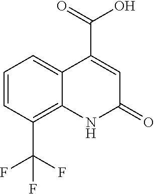 [SMILES: O=C(O)c1cc(=O)[nH]c2c(C(F)(F)F)cccc12]

To a flask containing 7-(trifluoromethyl)indoline-2,3-dione (1.0 g, 4.6 mmol) and malonic acid (1.5 g, 14.0 mmol) was added acetic acid (10 mL). The reaction was stirred at 110° C. for 40 h. The mixture was allowed to cool to r.t and water was added. The solid was collected by filtration and washed with water. The crude material was used in the next step without further purification. LCMS calculated for C₁₁H₇F₃NO₃ (M+H)⁺: m/z=258.0; found 258.0.

### Step 2. Ethyl 2-oxo-8-(trifluoromethyl)-4,2-dihydroquinoline-4-carboxylate

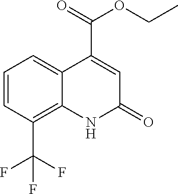 [SMILES: CCOC(=O)c1cc(=O)[nH]c2c(C(F)(F)F)cccc12]

To a suspension of 2-oxo-8-(trifluoromethyl)-1,2-dihydroquinoline-4-carboxylic acid (0.5 g, 2 mmol) in EtOH (10 mL), in an ice bath, was added thionyl chloride (3 mL) dropwise. The mixture was stirred at r.t for 15 min then refluxed for 2 h. The reaction mixture was cooled to r.t and the solvent was evaporated in vacuo. The crude material was used in the next step without further purification. LCMS calculated for C₁₃H₁₁F₃NO₃ (M+H)⁺: m/z=286.1; found 286.1.

### Step 3. Ethyl 2-chloro-8-(trifluoromethyl)quinoline-4-carboxylate

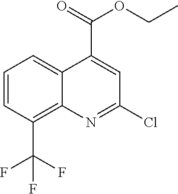 [SMILES: CCOC(=O)c1cc(Cl)nc2c(C(F)(F)F)cccc12]

A mixture of ethyl 2-oxo-8-(trifluoromethyl)-1,2-dihydroquinoline-4-carboxylate (570 mg, 2 mmol) and phosphoryl trichloride (6 mL) was stirred at 100° C. for 30 min. The reaction mixture was allowed to cool to r.t and concentrated in vacuo. The residue was quenched with ice water. The precipitated solid was then filtered and washed with water. The crude material was used in the next step without further purification. LCMS calculated for C₁₃H₁₀ClF₃NO₂ (M+H)⁺: m/z=304.0; found 304.0.

### Step 4. Ethyl 2-((2,4-dimethoxybenzyl)amino)-8-(trifluoromethyl)quinoline-4-carboxylate

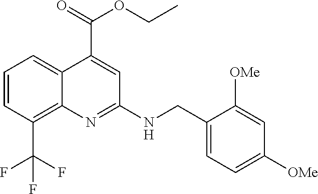 [SMILES: CCOC(=O)c1cc(NCc2ccc(OC)cc2OC)nc2c(C(F)(F)F)cccc12]

To a vial containing ethyl 2-chloro-8-(trifluoromethyl)quinoline-4-carboxylate (607 mg, 2 mmol) and (2,4-dimethoxyphenyl)methanamine (669 mg, 4 mmol) in DMSO (5 mL) was added triethylamine (0.7 mL, 5 mmol), which was then stirred at 110° C. for 1 h. The reaction mixture was cooled to r.t and water was added. The solid was then filtered and washed with water. The crude material was used in the next step without further purification. LCMS calculated for C₂₂H₂₂F₃N₂O₄(M+H)⁺: m/z=435.2; found 435.2.

### Step 5. 2-(2-Amino-8-(trifluoromethyl)quinolin-4-yl)propan-2-ol

To a vial containing ethyl 2-((2,4-dimethoxybenzyl)amino)-8-(trifluoromethyl)quinoline-4-carboxylate (870 mg, 2 mmol) in THF (10 ml) was added dropwise methylmagnesium bromide (4 ml, 3 M in Et₂O) at 0° C. The reaction was stirred at r.t for 15 min. The mixture was quenched with sat. NH₄Cl and extracted with EtOAc. The organic layers were combined, dried over Na₂SO₄, filtered, and the solvents were evaporated in vacuo. The crude material was then dissolved in TFA (5 mL) and stirred at 100° C. for 10 min. The reaction mixture was cooled to r.t, concentrated to remove volatiles, diluted with EtOAc and neutralized with sat. NaHCO₃. The organic layer was subsequently washed with water and brine, dried over Na₂SO₄, filtered, and the solvents were evaporated in vacuo. The crude material was purified by Biotage Isolera. LCMS calculated for C₁₃H₁₄F₃N₂O (M+H)⁺: m/z=271.1; found 271.1.

### Intermediate 3. 4-Chloro-8-(trifluoromethyl)quinolin-2-amine

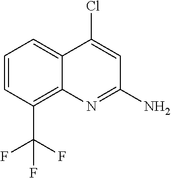 [SMILES: Nc1cc(Cl)c2cccc(C(F)(F)F)c2n1]

### Step 1. 3-Oxo-3-((2-(trifluoromethyl)phenyl)amino)propanoic acid

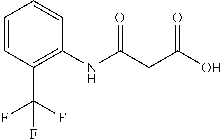 [SMILES: O=C(O)CC(=O)Nc1ccccc1C(F)(F)F]

To a reaction vial containing 2-(trifluoromethyl)aniline (0.65 g, 4 mmol), triethylamine (0.78 mL, 5.6 mmol) in DCM (12 mL) at 0° C. was added dropwise methyl 3-chloro-3-oxopropanoate (0.66 g, 4.8 mmol). The reaction was stirred at r.t for 1 h. The mixture was quenched with sat. NaHCO₃ and extracted with DCM. The organic layers were combined, dried over Na₂SO₄, filtered, and the solvents were evaporated in vacuo. The crude material was then dissolved in THF (10 mL), and aqueous solution of 2 M LiOH (10 mL) was then added. The reaction was stirred at r.t for 30 min. The mixture was acidified with 1 M HCl to pH 2 and extracted with EtOAc. The organic layer was subsequently washed with water and brine, dried over Na₂SO₄, filtered, and the solvents were evaporated in vacuo. The crude material was used in the next step without further purification. LCMS calculated for C₁₀H₉F₃NO₃ (M+H)⁺: m/z=248.1; found 248.1.

### Step 2. 4-Hydroxy-8-(trifluoromethyl)quinolin-2(1H)-one

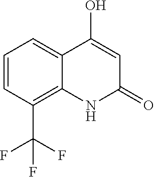 [SMILES: O=c1cc(O)c2cccc(C(F)(F)F)c2[nH]1]

A solution of 3-oxo-3-((2-(trifluoromethyl)phenyl)amino)propanoic acid (0.99 g, 4 mmol) in Eaton's reagent (15 mL) was stirred at 70° C. for 20 h. The reaction mixture was cooled to r.t and poured into crushed ice water. The solid precipitate was filtered and washed with water. The crude material was used in the next step without further purification. LCMS calculated for C₁₀H₇F₃NO₂ (M+H)⁺: m/z=230.0; found 230.0.

### Step 3. 2,4-Dichloro-8-(trifluoromethyl)quinoline

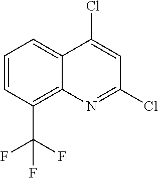 [SMILES: FC(F)(F)c1cccc2c(Cl)cc(Cl)nc12]

A solution of 4-hydroxy-8-(trifluoromethyl)quinolin-2(1H)-one (0.9 g, 4 mmol) in phosphoryl trichloride (15 ml) was refluxed with a condenser at 90° C. for 1 h. The reaction mixture was cooled to r.t and poured into crushed ice water. The solid precipitate was filtered and washed with water. The crude material was used in the next step without further purification. LCMS calculated for C₁₀H₅Cl₂F₃N (M+H)⁺: m/z=266.0; found 266.0.

### Step 4. 4-Chloro-8-(trifluoromethyl)quinolin-2-amine

A solution of 2,4-dichloro-8-(trifluoromethyl)quinoline (1 g, 3.76 mmol) in 1,4-dioxane (5 ml) and NH₄OH (5 mL) was microwaved for 1 h at 140° C. A mixture of two products was formed with approximately 1:1 ratio. The reaction mixture was cooled to r.t and diluted with EtOAc. The organic layer was subsequently washed with water and brine, dried over Na₂SO₄, filtered, and the solvents were evaporated in vacuo. The crude material was purified by Biotage Isolera with 4-chloro-8-(trifluoromethyl)quinolin-2-amine eluting at around 15% EtOAc/Hex. LCMS calculated for C₁₀H₇ClF₃N₂(M+H)⁺: m/z=247.0; found 247.0. 2-Chloro-8-(trifluoromethyl)quinolin-4-amine was eluted at 25% EtOAc/hex. LCMS calculated for C₁₀H₇ClF₃N₂(M+H)⁺: m/z=247.0; found 247.0.

### Example 1. Methyl ((1R,3R)-3-(6-((4-(hydroxymethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

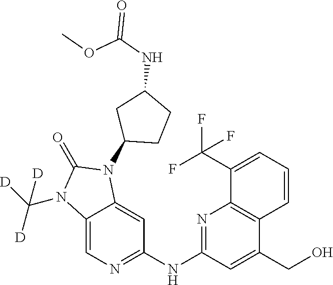 [SMILES: [2H]C([2H])([2H])n1c(=O)n([C@@H]2CC[C@@H](NC(=O)OC)C2)c2cc(Nc3cc(CO)c4cccc(C(F)(F)F)c4n3)ncc21]

To a solution of ethyl 2-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)-8-(trifluoromethyl)quinoline-4-carboxylate (12 mg, 0.021 mmol) in THF (0.5 mL) at 0° C., was added LiEt₃BH (0.10 mL, 0.10 mmol, 1 M in THF) dropwise. After 30 min, the reaction was quenched by the addition of MeOH (4 mL), acidified with TFA (0.2 mL), filtered, and purified via prep-LCMS (Xbridge C18 column, eluting with a gradient of acetonitrile/water containing 0.1% TFA, at flow rate of 60 mL/min) to afford the title compound. LCMS calculated for C₂₅H₂₃D₃F₃N₆O₄ (M+H)⁺: m/z=534.2; found 534.3. ¹H NMR (400 MHz, DMSO) δ 11.58 (s, 1H), 8.40 (s, 1H), 8.23 (d, J=8.3 Hz, 1H), 8.15 (d, J=7.4 Hz, 1H), 7.67 (s, 1H), 7.60 (t, J=7.9 Hz, 1H), 7.55 (s, 1H), 7.36 (d, J=7.1 Hz, 1H), 5.06 (s, 2H), 4.93 (p, J=8.6 Hz, 1H), 4.25 (q, J=6.8 Hz, 1H), 4.23 (br s, 1H), 3.55 (s, 3H), 2.34 (m, 1H), 2.28-2.02 (m, 3H), 1.95 (ddd, J=14.5, 9.6, 5.5 Hz, 1H), 1.64 (m, 1H).

### Example 2. Methyl ((1R,3R)-3-(6-((4-(1-hydroxyethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

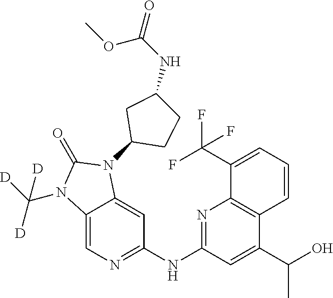 [SMILES: [2H]C([2H])([2H])n1c(=O)n([C@@H]2CC[C@@H](NC(=O)OC)C2)c2cc(Nc3cc(C(C)O)c4cccc(C(F)(F)F)c4n3)ncc21]

### Step 1. Methyl ((1R,3R)-3-(6-((4-formyl-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

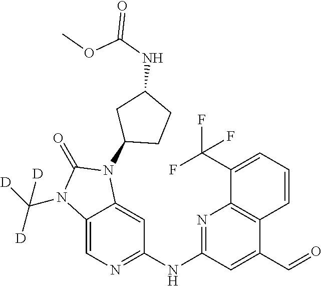 [SMILES: [2H]C([2H])([2H])n1c(=O)n([C@@H]2CC[C@@H](NC(=O)OC)C2)c2cc(Nc3cc(C=O)c4cccc(C(F)(F)F)c4n3)ncc21]

To a vial containing methyl ((1R,3R)-3-(6-((4-(hydroxymethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate (60 mg, 0.11 mmol) in DCM (2.5 mL) at 0° C., was added NaHCO₃ (47 mg, 0.56 mmol) and DMP (48 mg, 0.11 mmol). After 1 h, the reaction was quenched with saturated aqueous NaHCO₃ (5 mL) and diluted with DCM (5 mL). The layers were separated, and the aqueous layer was further extracted with DCM (3×3 mL). The combined organic layers were dried over Na₂SO₄, filtered, and concentrated in vacuo. The crude material was purified by Biotage Isolera to afford the title compound. LCMS calculated for C₂₅H₂₁D₃F₃N₆O₄ (M+H)⁺: m/z=532.2; found 532.2.

### Step 2. Methyl ((1R,3R)-3-(6-((4-(I-hydroxyethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

To a solution of methyl ((1R,3R)-3-(6-((4-formyl-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate (10 mg, 0.019 mmol) in THF (0.5 mL) at 0° C., was added methylmagnesium bromide (0.030 mL, 0.090 mmol, 3 M in Et₂O) dropwise. After 15 min, the reaction was quenched by the addition of MeOH (4 mL), acidified with TFA (0.2 mL), filtered, and purified via prep-LCMS (Xbridge C18 column, eluting with a gradient of acetonitrile/water containing 0.1% TFA, at flow rate of 60 mL/min) to afford the title compound. LCMS calculated for C₂₆H₂₅D₃F₃N₆O₄ (M+H)⁺: m/z=548.2; found 548.2. ¹H NMR (400 MHz, DMSO) δ 11.51 (s, 1H), 8.43-8.36 (m, 2H), 8.16 (d, J=7.4 Hz, 1H), 7.71-7.57 (d, J=6.6 Hz, 3H), 7.36 (d, J=7.1 Hz, 1H), 5.51 (q, J=6.4 Hz, 1H), 4.93 (p, J=8.3 Hz, 1H), 4.25 (q, J=6.9 Hz, 1H), 3.93 (br s, 1H), 3.55 (s, 3H), 2.34 (m, 1H), 2.25-2.02 (m, 3H), 2.01-1.89 (m, 1H), 1.69-1.58 (m, 1H), 1.48 (d, J=6.4 Hz, 3H).

### Example 3. Methyl ((1R,3R)-3-(3-(methyl-d₃)-2-oxo-6-((4-(1,1,1-trifluoro-2-hydroxypropan-2-yl)-8-(trifluoromethyl)quinolin-2-yl)amino)-2,3-dihydro-1H-imidazo[4,5-c]pyridin-1-yl)cyclopentyl)carbamate

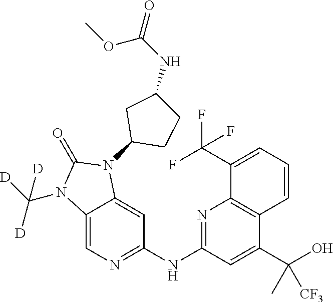 [SMILES: [2H]C([2H])([2H])n1c(=O)n([C@@H]2CC[C@@H](NC(=O)OC)C2)c2cc(Nc3cc(C(C)(O)C(F)(F)F)c4cccc(C(F)(F)F)c4n3)ncc21]

### Step 1. Methyl ((1R,3R)-3-(6-((4-acetyl-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

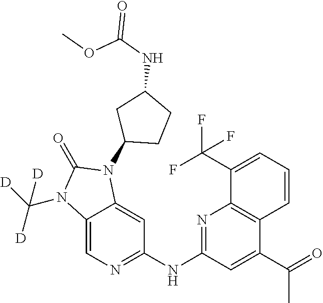 [SMILES: [2H]C([2H])([2H])n1c(=O)n([C@@H]2CC[C@@H](NC(=O)OC)C2)c2cc(Nc3cc(C(C)=O)c4cccc(C(F)(F)F)c4n3)ncc21]

To a solution of methyl ((1R,3R)-3-(6-((4-(hydroxymethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate (186 mg, 0.340 mmol) in DCM (5 mL) at 0° C., was added NaHCO₃ (143 mg, 1.70 mmol) and DMP (216 mg, 0.510 mmol). After 30 min the reaction was quenched with saturated aqueous NaHCO₃ (5 mL) and the layers separated. The aqueous layer was further extracted with DCM (3×3 mL) and the combined organic Layers were dried over Na₂SO₄, filtered, and concentrated in vacuo. The crude residue was used in the next step without further purification. LCMS calculated for C₂₆H₂₃D₃F₃N₆O₄ (M+H)⁺: m/z=546.2; found 546.3.

### Step 2. Methyl ((1R,3R)-3-(3-(methyl-d₃)-2-oxo-6-((4-(1,1,1-trifluoro-2-hydroxypropan-2-yl)-8-(trifluoromethyl)quinolin-2-yl)amino)-2,3-dihydro-1H-imidazo[4,5-c]pyridin-1-yl)cyclopentyl)carbamate

To a solution of methyl ((1R,3R)-3-(6-((4-acetyl-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate (10 mg, 0.018 mmol) in THF (0.5 mL) was added (trifluoromethyl)trimethylsilane (0.012 mL, 0.073 mmol) and TBAF (0.014 mL, 0.014 mmol, 1 M in THF). After 2 h, the reaction was diluted with MeOH (4 mL), acidified with TFA (0.2 mL), filtered, and purified via prep-LCMS (Xbridge C18 column, eluting with a gradient of acetonitrile/water containing 0.1% TFA, at flow rate of 60 mL/min) to afford the title compound. LCMS calculated for C₂₇H₂₄D₃F₆N₆O₄ (M+H)⁺: m/z=616.2; found 616.3.

### Example 4. Methyl ((1R,3R)-3-(3-(methyl-d₃)-6-((4-methyl-8-(trifluoromethyl)quinolin-2-yl)amino)-2-oxo-2,3-dihydro-1H-imidazo[4,5-c]pyridin-1-yl)cyclopentyl)carbamate

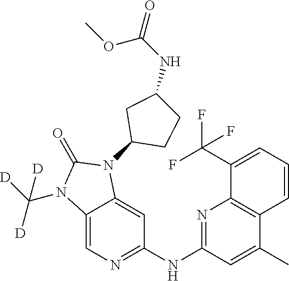 [SMILES: [2H]C([2H])([2H])n1c(=O)n([C@@H]2CC[C@@H](NC(=O)OC)C2)c2cc(Nc3cc(C)c4cccc(C(F)(F)F)c4n3)ncc21]

### Step 1. Tert-Butyl (tert-butoxycarbonyl)(4-chloro-8-(trifluoromethyl)quinolin-2-yl)carbamate

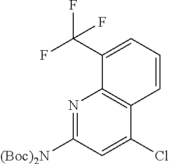

To a solution of Intermediate 3 (1.00 g, 4.05 mmol) in THF (40 mL) was added Et₃N (1.70 mL, 12.2 mmol), DMAP (0.074 g, 0.608 mmol) and Boc₂O (2.21 g, 10.1 mmol). The reaction mixture was stirred at ambient temperature overnight, after which the reaction was diluted with saturated aqueous NaHCO₃ (50 mL). The layers were separated, and the aqueous layer was further extracted with EtOAc (3×25 mL). The combined organic layers were dried over Na₂SO₄, filtered and concentrated in vacuo. The crude residue was used in the next step without further purification. LCMS calculated for C₂₀H₂₃ClF₃N₂O₄ (M+H)⁺: m/z=447.1; found 447.2.

### Step 2. 4-Methyl-8-(trifluoromethyl)quinolin-2-amine

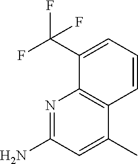 [SMILES: Cc1cc(N)nc2c(C(F)(F)F)cccc12]

To a vial containing tert-butyl (tert-butoxycarbonyl)(4-chloro-8-(trifluoromethyl)quinolin-2-yl)carbamate (40 mg, 0.090 mmol), [1,1′-bis(diphenylphosphino) ferrocene]dichloropalladium(II)-DCM adduct (15.0 mg, 0.018 mmol), Cs₂CO₃ (150 mg, 0.45 mmol) and 2,4,4,5,5-pentamethyl-1,3,2-dioxaborolane (51 mg, 0.36 mmol) was added 1,4-dioxane (1 mL) and the reaction mixture heated to 90° C. for 1 h. Upon completion, the reaction was cooled to ambient temperature, filtered, concentrated in vacuo and carried forward without any further purification (LCMS calculated for C₂₁H₂₆F₃N₂O₄(M+H)⁺: m/z=427.2; found 427.3). The crude residue was taken up in DCM/TFA (2 mL, 1:1 v/v). After 15 min, the reaction was concentrated in vacuo. The resultant residue was neutralized with saturated aqueous NaHCO₃ (5 mL) and extracted with DCM (4×2 mL). The combined organic layers were dried over Na₂SO₄, filtered, concentrated in vacuo and carried forward without further purification. LCMS calculated for C₁₁H₁₀F₃N₂(M+H)⁺: m/z=227.1; found 227.1.

### Step 3. Methyl ((1R,3R)-3-(3-(methyl-d3)-6-((4-methyl-8-(trifluoromethyl)quinolin-2-yl)amino)-2-oxo-2,3-dihydro-1H-imidazo[4,5-c]pyridin-1-yl)cyclopentyl)carbamate

To a vial containing Intermediate 1 (12 mg, 0.037 mmol), tris(dibenzylideneacetone) dipalladium(0) (7 mg, 0.0073 mmol), xantphos (9 mg, 0.015 mmol), Cs₂CO₃ (36 mg, 0.11 mmol) and 4-methyl-8-(trifluoromethyl)quinolin-2-amine (17 mg, 0.073 mmol) was added 1,4-dioxane (1 mL). The reaction was degassed by bubbling with N₂ for 5 min, after which it was heated to 85° C. for 6 h. After cooling the reaction to ambient temperature, it was diluted with acetonitrile (4 mL), acidified with TFA (0.2 mL), filtered, and purified via prep-LCMS (Xbridge C18 column, eluting with a gradient of acetonitrile/water containing 0.1% TFA, at flow rate of 60 mL/min) to afford the title compound. LCMS calculated for C₂₅H₂₃D₃F₃N₆O₃ (M+H)⁺: m/z=518.2; found 518.1. ¹H NMR (400 MHz, DMSO) δ 11.60 (s, 1H), 8.39 (s, 1H), 8.33 (d, J=8.3 Hz, 1H), 8.16 (d, J=7.4 Hz, 1H), 7.67-7.59 (m, 2H), 7.36 (d, J=7.0 Hz, 1H), 7.26 (s, 1H), 4.91 (p, J=8.4 Hz, 1H), 4.24 (q, J=6.8 Hz, 1H), 3.55 (s, 3H), 2.72 (s, 3H), 2.34 (m, 1H), 2.26-2.02 (m, 3H), 1.95 (m, 1H), 1.63 (m, 1H).

### Example 5. Methyl ((1R,3R)-3-(3-(methyl-d₃)-6-((4-(methylsulfinyl)-8-(trifluoromethyl)quinolin-2-yl)amino)-2-oxo-2,3-dihydro-1H-imidazo[4,5-c]pyridin-1-yl)cyclopentyl)carbamate

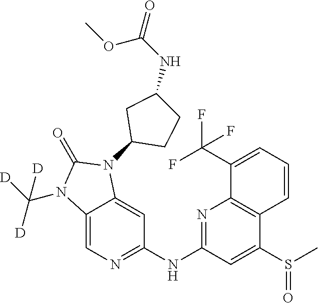 [SMILES: [2H]C([2H])([2H])n1c(=O)n([C@@H]2CC[C@@H](NC(=O)OC)C2)c2cc(Nc3cc(S(C)=O)c4cccc(C(F)(F)F)c4n3)ncc21]

### Step 1. Tert-Butyl (4-(methylthio)-8-(trifluoromethyl)quinolin-2-yl)carbamate

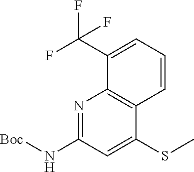 [SMILES: CSc1cc(NC(=O)OC(C)(C)C)nc2c(C(F)(F)F)cccc12]

To a solution of tert-butyl (tert-butoxycarbonyl)(4-chloro-8-(trifluoromethyl)quinolin-2-yl)carbamate (75.0 mg, 0.168 mmol) in DMF (2 mL) was added sodium methanethiolate (18 mg, 0.252 mmol) and the reaction heated to 70° C. After stirring at this temperature for 30 min, the reaction was cooled to ambient temperature and diluted with water (3 mL) and EtOAc (3 mL). The layers were separated, and the aqueous layer was further extracted with EtOAc (3×3 mL). The combined organic layers were washed with water (3×3 mL) and brine (3 mL). The organic layer was dried over Na₂SO₄, filtered, and concentrated in vacuo. The crude residue was carried forward without any further purification. LCMS calculated for C₁₆H₁₈F₃N₂O₂S (M+H)⁺: m/z=359.1; found 359.0.

### Step 2. 4-(Methylsulfinyl)-8-(trifluoromethyl)quinolin-2-amine

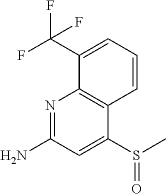 [SMILES: CS(=O)c1cc(N)nc2c(C(F)(F)F)cccc12]

To a vial containing tert-butyl (4-(methylthio)-8-(trifluoromethyl)quinolin-2-yl)carbamate (60 mg, 0.168 mmol) in DMF (2 mL) was added m-CPBA (29 mg, 0.168 mmol) and the reaction was stirred at ambient temperature. After 30 min, the reaction was quenched with saturated aqueous Na₂S₂O₃ (3 mL) and diluted with EtOAc (3 mL). The layers were separated, and the aqueous layer was further extracted with EtOAc (3×3 mL). The combined organic layers were washed with water (3×3 mL) and brine (3 mL). The organic layer was dried over Na₂SO₄, filtered, and concentrated in vacuo. The crude sulfoxide was carried forward without further purification (LCMS calculated for C₁₆H₁₈F₃N₂O₃S (M+H)⁺: m/z=375.1; found 375.2). The crude residue was taken up in DCM/TFA (2 mL, 1:1 v/v) and stirred at ambient temperature. After 15 min, the reaction was concentrated in vacuo, neutralized with saturated aqueous NaHCO₃ (5 mL) and extracted with DCM (3×3 mL). The combined organic layers were dried over Na₂SO₄, filtered, and concentrated in vacuo. The crude residue was carried forward without further purification. LCMS calculated for C₁₁H₁₀F₃N₂OS (M+H)⁺: m/z=275.1; found 275.2.

### Step 3. Methyl ((1R,3R)-3-(3-(methyl-d₃)-6-((4-(methylsulfinyl)-8-(trifluoromethyl)quinolin-2-yl)amino)-2-oxo-2,3-dihydro-1H-imidazo[4,5-c]pyridin-1-yl)cyclopentyl)carbamate

This compound was prepared according to the procedure described in Example 4, Step 3, using 4-(methylsulfinyl)-8-(trifluoromethyl)quinolin-2-amine in place of 4-methyl-8-(trifluoromethyl)quinolin-2-amine. LCMS calculated for C₂₅H₂₃D₃F₃N₆O₄S (M+H)⁺: m/z=566.2; found 566.2. ¹H NMR (400 MHz, DMSO) δ 11.65 (s, 1H), 8.41 (s, 1H), 8.24 (d, J=7.5 Hz, 1H), 8.19-8.11 (m, 2H), 7.66 (t, J=7.9 Hz, 1H), 7.34 (d, J=7.5 Hz, 1H), 6.36 (s, 1H), 4.96-4.82 (m, 1H), 4.30-4.14 (m, 1H), 3.54 (s, 3H), 2.95 (s, 3H), 2.41-1.90 (m, 4H), 1.86-1.75 (m, 1H), 1.58-1.48 (m, 1H).

### Examples 6-7. Methyl ((1R,3R)-3-(3-(methyl-d₃)-6-((4-(methylsulfonyl)-8-(trifluoromethyl)quinolin-2-yl)amino)-2-oxo-2,3-dihydro-1H-imidazo[4,5-c]pyridin-1-yl)cyclopentyl)carbamate and methyl ((1R,3R)-3-(3-(methyl-d₃)-6-((4-(S-methylsulfonimidoyl)-8-(trifluoromethyl)quinolin-2-yl)amino)-2-oxo-2,3-dihydro-1H-imidazo[4,5-c]pyridin-1-yl)cyclopentyl)carbamate

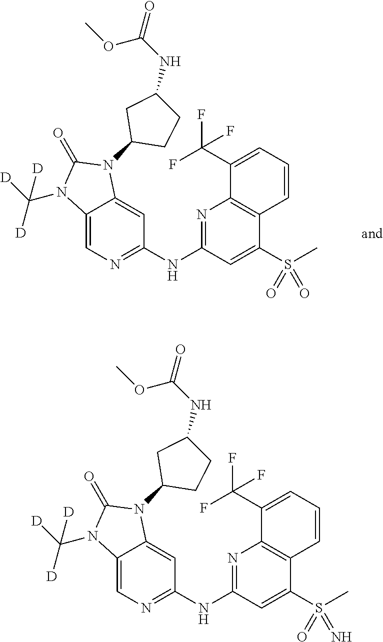 [SMILES: [2H]C([2H])([2H])n1c(=O)n([C@@H]2CC[C@@H](NC(=O)OC)C2)c2cc(Nc3cc(S(C)(=O)=O)c4cccc(C(F)(F)F)c4n3)ncc21 ; [2H]C([2H])([2H])n1c(=O)n([C@@H]2CC[C@@H](NC(=O)OC)C2)c2cc(Nc3cc(S(C)(=N)=O)c4cccc(C(F)(F)F)c4n3)ncc21]

### Step 1. 4-(Methylsulfonyl)-8-(trifluoromethyl)quinolin-2-amine & (2-amino-8-(trifluoromethyl)quinolin-4-yl)(imino)(methyl)-λ⁶-sulfanone

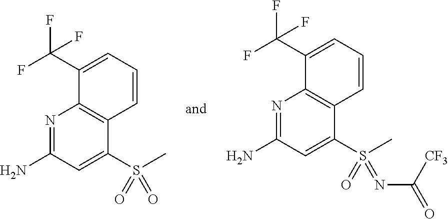 [SMILES: CS(=O)(=O)c1cc(N)nc2c(C(F)(F)F)cccc12 ; CS(=O)(=NC(=O)C(F)(F)F)c1cc(N)nc2c(C(F)(F)F)cccc12]

To a vial containing 4-(methylsulfinyl)-8-(trifluoromethyl)quinolin-2-amine (Example 5, Step 2) (30 mg, 0.11 mmol) as a solution in DCM (1 mL) was added 2,2,2-trifluoroacetamide (25 mg, 0.22 mmol), MgO (18 mg, 0.45 mmol) and rhodium(II) acetate dimer (3 mg, 0.0056 mmol). The reaction was stirred overnight under N₂. The reaction was quenched with water (1 mL) and the layers separated. The aqueous layer was further extracted with DCM (3×2 mL). The combined organic layers were dried over Na₂SO₄, filtered, and concentrated in vacuo. The crude material was purified by Biotage Isolera to afford a mixture of the title compounds. LCMS calculated for C₁₁H₁₀F₃N₂O₂S (M+H)⁺: m/z=291.0; found 291.0. LCMS calculated for C₁₃H₁₀F₆N₃O₂S (M+H)⁺: m/z=386.0; found 386.0.

### Step 2. Methyl ((1R,3R)-3-(3-(methyl-d₃)-6-((4-(methylsulfonyl)-8-(trifluoromethyl)quinolin-2-yl)amino)-2-oxo-2,3-dihydro-1H-imidazo[4,5-c]pyridin-1-yl)cyclopentyl)carbamate & methyl ((1R,33R)-3-(3-(methyl-d₃)-6-((4-(S-methylsulfonimidoyl)-8-(trifluoromethyl)quinolin-2-yl)amino)-2-oxo-2,3-dihydro-1H-imidazo[4,5-c]pyridin-1-yl)cyclopentyl)carbamate

The title compounds were prepared according to the procedure described in Example 4, Step 3, using a mixture of 4-(methylsulfonyl)-8-(trifluoromethyl)quinolin-2-amine and (2-amino-8-(trifluoromethyl)quinolin-4-yl)(imino)(methyl)-λ⁶-sulfanone in place of 4-methyl-8-(trifluoromethyl)quinolin-2-amine. The mixture of products was separated via prep-LCMS (Xbridge C18 column, eluting with a gradient of acetonitrile/water containing 0.1% TFA, at flow rate of 60 mL/min).

Example 6: LCMS calculated for C₂₅H₂₃D₃F₃N₆O₅S (M+H)⁺: m/z=582.2; found 582.3. ¹H NMR (400 MHz, DMSO) δ 10.81 (s, 1H), 8.67 (d, J=8.4 Hz, 1H), 8.39 (s, 1H), 8.24-8.15 (m, 2H), 7.64 (t, J=7.9 Hz, 1H), 7.30 (d, J=6.9 Hz, 1H), 4.81 (p, J=8.2 Hz, 1H), 4.26 (q, J=7.0 Hz, 1H), 3.86 (br s, 1H), 3.53 (s, 3H), 3.47 (s, 3H), 2.41 (m, 1H), 2.20 (m, 1H), 2.11 (q, J=7.9 Hz, 2H), 1.92 (m, 1H), 1.60 (q, J=9.6 Hz, 1H).

Example 7: LCMS calculated for C₂₅H₂₄D₃F₃N₇O₄S (M+H)⁺: m/z=581.2; found 581.3. ¹H NMR (400 MHz, DMSO) δ 10.71 (s, 1H), 8.66 (d, J=8.5 Hz, 1H), 8.44 (s, 1H), 8.24-8.13 (m, 3H), 7.62 (t, J=8.0 Hz, 1H), 6.51 (s, 1H), 4.81 (d, J=7.9 Hz, 1H), 4.31-4.23 (m, 1H), 3.88 (s, 1H), 3.53 (s, 3H), 3.47 (s, 3H), 2.24-2.15 (m, 1H), 2.14-2.06 (m, 2H), 2.04-1.87 (m, 2H), 1.58 (d, J=12.8 Hz, 1H).

### Example 8. Benzyl ((1R,3R)-3-(3-(methyl-d₃)-2-oxo-6-((8-(trifluoromethyl)quinolin-2-yl)amino)-2,3-dihydro-1H-imidazo[4,5-c]pyridin-1-yl)cyclopentyl)carbamate

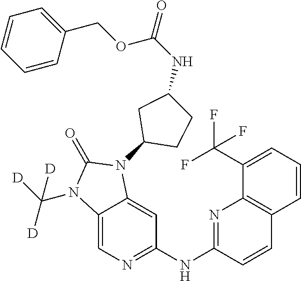 [SMILES: [2H]C([2H])([2H])n1c(=O)n([C@@H]2CC[C@@H](NC(=O)OCc3ccccc3)C2)c2cc(Nc3ccc4cccc(C(F)(F)F)c4n3)ncc21]

### Step 1. Tert-Butyl ((1R,3R)-3-(3-(methyl-d3)-2-oxo-6-((8-(trifluoromethyl)quinolin-2-yl)amino)-2,3-dihydro-1H-imidazo[4,5-c]pyridin-1-yl)cyclopentyl)carbamate

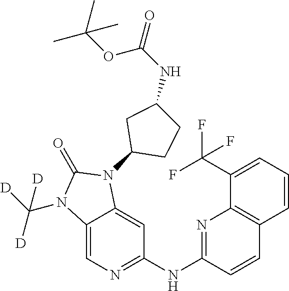 [SMILES: [2H]C([2H])([2H])n1c(=O)n([C@@H]2CC[C@@H](NC(=O)OC(C)(C)C)C2)c2cc(Nc3ccc4cccc(C(F)(F)F)c4n3)ncc21]

This compound was prepared according to the procedure described in Example 4, Step 3, using tert-butyl ((1R,3R)-3-(6-chloro-3-(methyl-d₃)-2-oxo-2,3-dihydro-1H-imidazo[4,5-c]pyridin-1-yl)cyclopentyl)carbamate in place of Intermediate 1, and 8-(trifluoromethyl)quinolin-2-amine in place of 4-methyl-8-(trifluoromethyl)quinolin-2-amine. LCMS calculated for C₂₇H₂₇D₃F₃N₆O₃ (M+H)⁺: m/z=546.3; found 546.3.

### Step 2. Benzyl ((1R,3R)-3-(3-(methyl-d₃)-2-oxo-6-((8-(trifluoromethyl)quinolin-2-yl)amino)-2,3-dihydro-1H-imidazo[4,5-c]pyridin-1-yl)cyclopentyl)carbamate

To a vial containing a solution of tert-butyl ((1R,3R)-3-(3-(methyl-d₃)-2-oxo-6-((8-(trifluoromethyl)quinolin-2-yl)amino)-2,3-dihydro-1H-imidazo[4,5-c]pyridin-1-yl)cyclopentyl)carbamate (312 mg, 0.572 mmol) in DCM/MeOH (10 mL, 1:1 v/v) was added 4 N HCl in 1,4-dioxane (5 mL). The reaction mixture was stirred for 30 min at ambient temperature, after which time it was concentrated in vacuo and the crude residue carried forward without any further purification (LCMS calculated for C₂₂H₁₉D₃F₃N₆O (M+H)⁺: m/z=446.2; found 446.2). To vial containing a solution of the crude amine (10 mg, 0.022 mmol) in DMF (0.5 mL) was added Et₃N (0.010 mL, 0.067 mmol) and benzyl chloroformate (0.0065 mL, 0.045 mmol). The reaction mixture was stirred at ambient temperature for 30 min, after which it was diluted with acetonitrile (4 mL), acidified with TFA (0.2 mL), filtered, and purified via prep-LCMS (Xbridge C18 column, eluting with a gradient of acetonitrile/water containing 0.1% TFA, at flow rate of 60 mL/min) to afford the title compound. LCMS calculated for C₃₀H₂₅D₃F₃N₆O₃ (M+H)⁺: m/z=580.2; found 580.3. ¹H NMR (400 MHz, DMSO) δ 11.41 (s, 1H), 8.45 (d, J=9.0 Hz, 1H), 8.37 (s, 1H), 8.21 (d, J=8.0 Hz, 1H), 8.14 (d, J=7.4 Hz, 1H), 7.60 (t, J=7.7 Hz, 1H), 7.51 (d, J=6.9 Hz, 1H), 7.45 (d, J=9.0 Hz, 1H), 7.37 (m, 5H), 5.03 (s, 2H), 4.92 (p, J=8.4 Hz, 1H), 4.59 (s, 1H), 4.28 (q, J=6.9 Hz, 1H), 2.37 (m, 1H), 2.29-2.18 (m, 1H), 2.17-2.02 (m, 2H), 1.96 (m, 1H), 1.64 (m, 1H).

### Example 9. Methyl ((1R,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

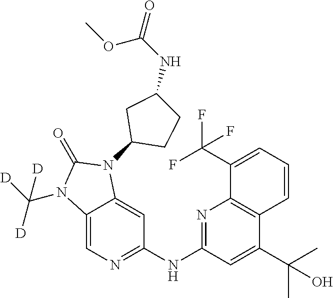 [SMILES: [2H]C([2H])([2H])n1c(=O)n([C@@H]2CC[C@@H](NC(=O)OC)C2)c2cc(Nc3cc(C(C)(C)O)c4cccc(C(F)(F)F)c4n3)ncc21]

A reaction vial containing methyl ((1R,3R)-3-(6-chloro-3-(methyl-d₃)-2-oxo-2,3-dihydro-1H-imidazo[4,5-c]pyridin-1-yl)cyclopentyl)carbamate (33 mg, 0.1 mmol), 2-(2-amino-8-(trifluoromethyl)quinolin-4-yl)propan-2-ol (32 mg, 0.12 mmol), xantphos-Pd-G3 (10 mg, 0.01 mmol) and cesium carbonate (99 mg, 0.3 mmol) was evacuated and backfilled with nitrogen. 1,4-Dioxane (1.5 mL) was added to the reaction mixture, then it was then stirred at 110° C. for 3 h. Upon completion, the reaction was diluted with DCM, filtered through Celite and concentrated in vacuo. The crude material was diluted with acetonitrile and purified by prep-LCMS (Xbridge C18 column, eluting with a gradient of acetonitrile/water containing 0.1% TFA, at flow rate of 60 mL/min). LCMS calculated for C₂₇H₂₇D₃F₃N₆O₄ (M+H)⁺: m/z=562.3; found 562.3. ¹H NMR (400 MHz, DMSO) δ 10.06 (s, 1H), 8.96 (d, J=8.5 Hz, 1H), 8.61 (s, 1H), 8.10 (s, 1H), 7.97 (d, J=7.4 Hz, 1H), 7.56 (s, 1H), 7.41 (t, J=7.9 Hz, 1H), 7.28 (d, J=7.1 Hz, 1H), 5.57 (s, 1H), 4.80 (m, 1H), 4.27 (m, 1H), 3.54 (s, 3H), 2.43 (m, 1H), 2.28-2.17 (m, 1H), 2.17-2.06 (m, 2H), 1.99-1.86 (m, 1H), 1.68 (s, 6H), 1.64-1.52 (m, 1H).

### Example 10. Methyl ((1R,3R)-3-(6-((4-(isopropyl(methyl)carbamoyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

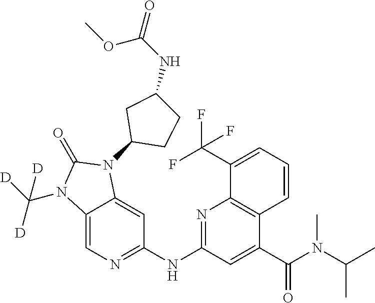 [SMILES: [2H]C([2H])([2H])n1c(=O)n([C@@H]2CC[C@@H](NC(=O)OC)C2)c2cc(Nc3cc(C(=O)N(C)C(C)C)c4cccc(C(F)(F)F)c4n3)ncc21]

### Step 1. Ethyl 2-((1-((1R,3R)-3-((methoxycarbonyl)amino)cyclopentyl)-3-(methyl-d)-2-oxo-2,3-dihydro-1H-imidazo[4,5-c]pyridin-6-yl)amino)-8-(trifluoromethyl)quinoline-4-carboxylate

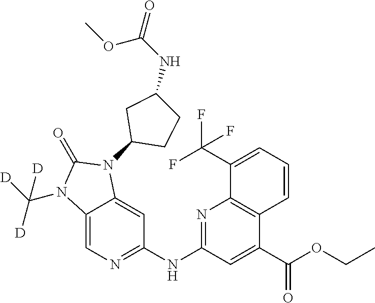 [SMILES: [2H]C([2H])([2H])n1c(=O)n([C@@H]2CC[C@@H](NC(=O)OC)C2)c2cc(Nc3cc(C(=O)OCC)c4cccc(C(F)(F)F)c4n3)ncc21]

A reaction vial containing methyl ((1R,3R)-3-(6-chloro-3-(methyl-d₃)-2-oxo-2,3-dihydro-1H-imidazo[4,5-c]pyridin-1-yl)cyclopentyl)carbamate (656 mg, 2 mmol), ethyl 2-amino-8-(trifluoromethyl)quinoline-4-carboxylate (625 mg, 2.2 mmol), xantphos-Pd-G3 (189 mg, 0.2 mmol) and cesium carbonate (1.9 g, 6 mmol) was evacuated and backfilled with nitrogen. 1,4-Dioxane (15 mL) was added to the reaction mixture, which was then stirred at 110° C. for 3 h. The reaction mixture was cooled to r.t and diluted with EtOAc. The organic layer was subsequently washed with water and brine, dried over Na₂SO₄, filtered, and the solvents were evaporated in vacuo. The crude material was purified by Biotage Isolera. LCMS calculated for C₂₇H₂₅D₃F₃N₆O₅ (M+H)⁺: m/z=576.2; found 576.2.

### Step 2. 2-((1-((1R,3R)-3-((Methoxycarbonyl)amino)cyclopentyl)-3-(methyl-d₃)-2-oxo-2,3-dihydro-1H-imidazo[4,5-c]pyridin-6-yl)amino)-8-(trifluoromethyl)quinoline-4-carboxylic acid

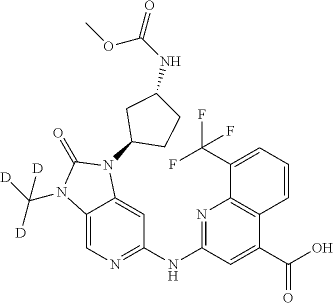 [SMILES: [2H]C([2H])([2H])n1c(=O)n([C@@H]2CC[C@@H](NC(=O)OC)C2)c2cc(Nc3cc(C(=O)O)c4cccc(C(F)(F)F)c4n3)ncc21]

To a reaction vial containing ethyl 2-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)-8-(trifluoromethyl)quinoline-4-carboxylate (800 mg, 1.4 mmol) in THF (10 mL) was added aqueous solution of 1 M LiOH (5.6 mL, 5.6 mmol) and stirred at r.t for 1 h. The mixture was concentrated in vacuo, then the solid was collected by filtration and washed with water. The crude material was used in the next step without further purification. LCMS calculated for C₂₅H₂₁D₃F₃N₆O₅ (M+H)⁺: m/z=548.2; found 548.2.

### Step 3. Methyl ((1R,3R)-3-(6-((4-(isopropyl(methyl)carbamoyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

To a reaction vial containing 2-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)-8-(trifluoromethyl)quinoline-4-carboxylic acid (8 mg, 0.015 mmol), HATU (11 mg, 0.029 mg) in DMF (0.5 mL) was added DIPEA (6 mg, 0.044 mmol), followed by N-methylpropan-2-amine (4 mg, 0.05 mmol). The mixture was stirred at 60° C. for 15 min. The reaction mixture was allowed to cool to r.t, diluted with acetonitrile and purified by prep-LCMS (Xbridge C18 column, eluting with a gradient of acetonitrile/water containing 0.1% TFA, at flow rate of 60 mL/min). LCMS calculated for C₂₉H₃₀D₃F₃N₇O₄(M+H)⁺: m/z=603.4; found 603.4. ¹H NMR (400 MHz, DMSO) δ 11.14 (s, 1H), 8.35 (s, 1H), 8.18 (d, J=7.4 Hz, 1H), 7.98 (s, 1H), 7.91 (d, J=8.2 Hz, 0.7H), 7.85 (d, J=8.2 Hz, 0.3H), 7.60 (d, J=7.9 Hz, 1H), 7.34 (s, 1H), 7.32 (s, 1H), 4.89 (p, J=7.9 Hz, 1H), 4.25 (p, J=6.9 Hz, 1H), 3.65 (p, J=6.6 Hz, 1H), 3.55 (s, 3H), 3.01 (s, 2H), 2.67 (s, 1H), 2.45-2.32 (m, 1H), 2.29-2.16 (m, 1H), 2.17-2.04 (m, 2H), 2.01-1.86 (m, 1H), 1.72-1.52 (m, 1H), 1.27 (d, J=6.8 Hz, 2H), 1.15 (d, J=6.5 Hz, 2H), 1.05 (d, J=6.6 Hz, 2H).

### Example 11. Methyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl-1-d)carbamate

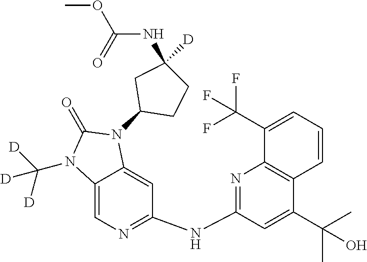 [SMILES: [2H]C([2H])([2H])n1c(=O)n([C@@H]2CC[C@]([2H])(NC(=O)OC)C2)c2cc(Nc3cc(C(C)(C)O)c4cccc(C(F)(F)F)c4n3)ncc21]

The title compound was prepared according to the procedures described in Example 9, with methyl ((1S,3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-chloro-3-(methyl-d₃)-2-oxo-2,3-dihydro-1H-imidazo[4,5-c]pyridin-1-yl)cyclopentyl)carbamate. LCMS calculated for C₂₇H₂₆D₄F₃N₆O₄ (M+H)⁺: m/z=563.3; found: 563.3. ¹H NMR (400 MHz, DMSO) δ 11.31 (s, 1H), 9.00 (d, J=8.6 Hz, 1H), 8.37 (s, 1H), 8.12 (d, J=7.6 Hz, 1H), 7.78 (s, 1H), 7.58 (s, 1H), 7.54 (s, 1H), 7.35 (s, 1H), 5.74 (s, 1H), 4.92 (m, 1H), 3.55 (s, 3H), 2.41-2.31 (m, 1H), 2.28-2.03 (m, 3H), 1.97-1.89 (m, 1H), 1.72 (s, 6H), 1.68-1.54 (m, 1H).

### Example 12. Methyl ((1R,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl-1-d)carbamate

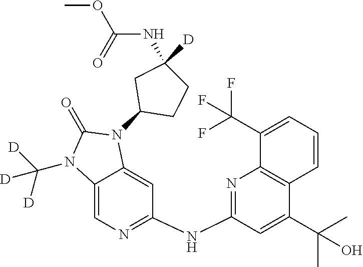 [SMILES: [2H]C([2H])([2H])n1c(=O)n([C@@H]2CC[C@@]([2H])(NC(=O)OC)C2)c2cc(Nc3cc(C(C)(C)O)c4cccc(C(F)(F)F)c4n3)ncc21]

The title compound was prepared according to the procedures described in Example 9, with methyl ((1R,3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-chloro-3-(methyl-d₃)-2-oxo-2,3-dihydro-1H-imidazo[4,5-c]pyridin-1-yl)cyclopentyl)carbamate. LCMS calculated for C₂₇H₂₆D₄F₃N₆O₄ (M+H)⁺: m/z=563.3; found: 563.3. ¹H NMR (400 MHz, DMSO) δ 11.40 (s, 1H), 9.01 (d, J=8.6 Hz, 1H), 8.39 (s, 1H), 8.12 (d, J=7.4 Hz, 1H), 7.95 (s, 1H), 7.59 (s, 1H), 7.56 (s, 1H), 7.42 (s, 1H), 5.73 (s, 1H), 4.98-4.54 (m, 1H), 3.55 (s, 3H), 2.35-1.92 (m, 5H), 1.89-1.79 (m, 1H), 1.72 (s, 6H).

### Example 13. Methyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate

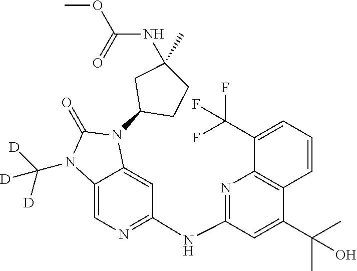 [SMILES: [2H]C([2H])([2H])n1c(=O)n([C@@H]2CC[C@](C)(NC(=O)OC)C2)c2cc(Nc3cc(C(C)(C)O)c4cccc(C(F)(F)F)c4n3)ncc21]

### Step 1. Tert-Butyl ((1S,3R)-3-(6-chloro-3-(methyl-d₃)-2-oxo-2,3-dihydro-1H-imidazo[4,5-c]pyridin-1-yl)-1-methylcyclopentyl)carbamate

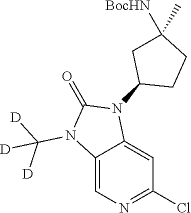 [SMILES: [2H]C([2H])([2H])n1c(=O)n([C@@H]2CC[C@](C)(NC(=O)OC(C)(C)C)C2)c2cc(Cl)ncc21]

The title compound was prepared according to the procedures described in Intermediate 1, Step 1-4 with tert-butyl ((1S,3R)-3-amino-1-methylcyclopentyl)carbamate replacing tert-butyl ((1R,3R)-3-aminocyclopentyl)carbamate. LCMS calculated for C₁₈H₂₃D₃ClN₄O₃(M+H)⁺: m/z=384.2; found: 384.2.

### Step 2. Tert-Butyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate

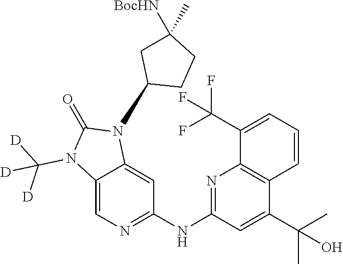 [SMILES: [2H]C([2H])([2H])n1c(=O)n([C@@H]2CC[C@](C)(NC(=O)OC(C)(C)C)C2)c2cc(Nc3cc(C(C)(C)O)c4cccc(C(F)(F)F)c4n3)ncc21]

A reaction vial containing tert-butyl ((1S,3R)-3-(6-chloro-3-(methyl-d₃)-2-oxo-2,3-dihydro-1H-imidazo[4,5-c]pyridin-1-yl)-1-methylcyclopentyl)carbamate (682 mg, 2 mmol), 2-(2-amino-8-(trifluoromethyl)quinolin-4-yl)propan-2-ol (594 mg, 2.2 mmol), xantphos-Pd-G3 (189 mg, 0.2 mmol) and cesium carbonate (1.9 g, 6 mmol) was evacuated and backfilled with nitrogen. 1,4-Dioxane (15 mL) was added to the reaction mixture, which was then stirred at 110° C. for 3 h. The reaction mixture was cooled to r.t and diluted with EtOAc. The organic layer was subsequently washed with water and brine, dried over Na₂SO₄, filtered, and the solvents were evaporated in vacuo. The crude material was purified by Biotage Isolera. LCMS calculated for C₃₁H₃₅D₃F₃N₆O₄ (M+H)⁺: m/z=618.3; found 618.3.

### Step 3. 1-((1R,3S)-3-Amino-3-methylcyclopentyl)-6-((4-(2-hydroxypropan-2-yl)-8-(trifluoromethyl)quinolin-2-yl)amino)-3-(methyl-d₃)-1,3-dihydro-2H-imidazo[4,5-c]pyridin-2-one

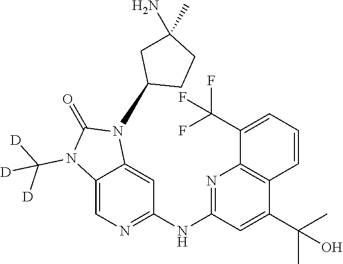 [SMILES: [2H]C([2H])([2H])n1c(=O)n([C@@H]2CC[C@](C)(N)C2)c2cc(Nc3cc(C(C)(C)O)c4cccc(C(F)(F)F)c4n3)ncc21]

To a reaction vial containing tert-butyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate (140 mg, 0.226 mmol) was added DCM (1 mL) and trifluoroacetic acid (0.5 mL). The reaction mixture was then stirred at r.t for 1 h. The reaction mixture was concentrated in vacuo and the crude material was used directly for next step. LCMS calculated for C₂₆H₂₇D₃F₃N₆O₂ (M+H)⁺: m/z=518.3; found 518.3.

### Step 4. Methyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate

To a reaction vial containing 1-((1R,3S)-3-amino-3-methylcyclopentyl)-6-((4-(2-hydroxypropan-2-yl)-8-(trifluoromethyl)quinolin-2-yl)amino)-3-(methyl-d₃)-1,3-dihydro-2H-imidazo[4,5-c]pyridin-2-one (10 mg, 0.02 mmol) and DIPEA (36 μL, 0.2 mmol) in DCM (0.5 mL) and MeOH (0.1 mL) was added methyl chloroformate (2 μL, 0.025 mmol). The reaction mixture was stirred at r.t for 20 min, then quenched with MeOH (2 ml) and solvents were evaporated in vacuo. The crude material was diluted with acetonitrile and purified by prep-LCMS (Xbridge C18 column, eluting with a gradient of acetonitrile/water containing 0.1% TFA, at flow rate of 60 mL/min). LCMS calculated for C₂₈H₂₉D₃F₃N₆O₄ (M+H)⁺: m/z=576.3; found: 576.3. ¹H NMR (400 MHz, DMSO-d₆) δ 11.26 (s, 1H), 9.00 (d, J=8.6 Hz, 1H), 8.37 (s, 1H), 8.12 (d, J=7.4 Hz, 1H), 7.99 (s, 1H), 7.58 (t, J=8.1 Hz, 1H), 7.54 (s, 1H), 7.44 (s, 1H), 5.72 (s, 1H), 4.81 (p, J=8.7 Hz, 1H), 3.53 (s, 3H), 2.51-2.36 (m, 1H), 2.38-2.20 (m, 2H), 2.21-2.02 (m, 2H), 1.86-1.78 (m, 1H), 1.72 (s, 6H), 1.40 (s, 3H).

### Example 14. 2-Methoxyethyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate

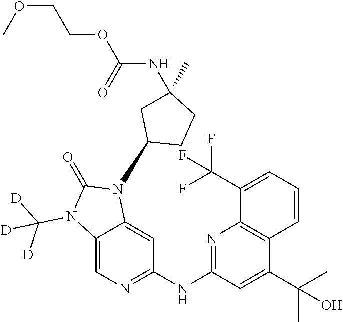 [SMILES: [2H]C([2H])([2H])n1c(=O)n([C@@H]2CC[C@](C)(NC(=O)OCCOC)C2)c2cc(Nc3cc(C(C)(C)O)c4cccc(C(F)(F)F)c4n3)ncc21]

The title compound was prepared according to the procedures described in Example 13, Step 4, with 2-methoxyethyl carbonochloridate replacing methyl chloroformate. LCMS calculated for C₃₀H₃₃D₃F₃N₆O₅ (M+H)⁺: m/z=620.3; found: 620.3.

### Example 15. Methyl ((trans)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclobutyl)carbamate

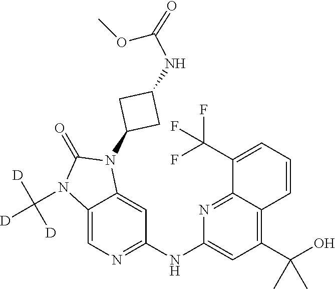 [SMILES: [2H]C([2H])([2H])n1c(=O)n([C@H]2C[C@H](NC(=O)OC)C2)c2cc(Nc3cc(C(C)(C)O)c4cccc(C(F)(F)F)c4n3)ncc21]

### Step 1. 1-((trans)-3-Aminocyclobutyl)-6-((4-(2-hydroxypropan-2-yl)-8-(trifluoromethyl)quinolin-2-yl)amino)-3-(methyl-d₃)-1,3-dihydro-2H-imidazo[4,5-c]pyridin-2-one

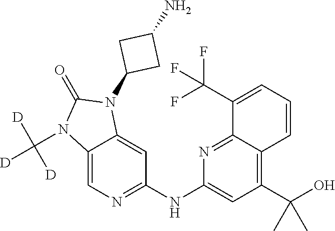 [SMILES: [2H]C([2H])([2H])n1c(=O)n([C@H]2C[C@H](N)C2)c2cc(Nc3cc(C(C)(C)O)c4cccc(C(F)(F)F)c4n3)ncc21]

The title compound was prepared according to the procedures described in Example 13, Step 1-3, with tert-butyl ((trans)-3-aminocyclobutyl)carbamate replacing tert-butyl ((1S,3R)-3-amino-1-methylcyclopentyl)carbamate. LCMS calculated for C₂₄H₂₃D₃F₃N₆O₂ (M+H)⁺: m/z=490.3; found: 490.3.

### Step 2. Methyl ((trans)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclobutyl)carbamate

The title compound was prepared according to the procedures described in Example 13, Step 4, with 1-((trans)-3-aminocyclobutyl)-6-((4-(2-hydroxypropan-2-yl)-8 (trifluoromethyl)quinolin-2-yl)amino)-3-(methyl-d₃)-1,3-dihydro-2H-imidazo[4,5-c]pyridin-2-one replacing 1-((1R,3S)-3-Amino-3-methylcyclopentyl)-6-((4-(2-hydroxypropan-2-yl)-8-(trifluoromethyl)quinolin-2-yl)amino)-3-(methyl-d₃)-1,3-dihydro-2H-imidazo[4,5-c]pyridin-2-one. LCMS calculated for C₂₆H₂₅D₃F₃N₆O₄ (M+H)⁺: m/z=548.3; found: 548.3.

### Example 16. Cyclopropyl ((trans)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclobutyl)carbamate

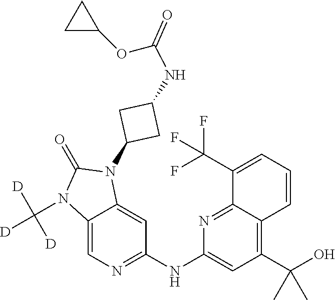 [SMILES: [2H]C([2H])([2H])n1c(=O)n([C@H]2C[C@H](NC(=O)OC3CC3)C2)c2cc(Nc3cc(C(C)(C)O)c4cccc(C(F)(F)F)c4n3)ncc21]

Pyridine (5 μL, 0.061 mmol) was added to a solution of cyclopropanol (2.4 mg, 0.041 mmol) and bis(2,5-dioxopyrrolidin-1-yl) carbonate (10.5 mg, 0.041 mmol) and then the mixture was stirred at 40° C. for 2 h. The reaction was cooled to r.t. and then the mixture was added to a solution of 1-((trans)-3-aminocyclobutyl)-6-((4-(2-hydroxypropan-2-yl)-8 (trifluoromethyl)quinolin-2-yl)amino)-3-(methyl-d₃)-1,3-dihydro-2H-imidazo[4,5-c]pyridin-2-one (10 mg, 0.020 mmol) and triethylamine (11 μl, 0.082 mmol) in DCM (0.500 ml). The reaction mixture was stirred at r.t for 30 min, then quenched with MeOH (2 ml) and solvents were evaporated in vacuo. The crude material was diluted with acetonitrile and purified by prep-LCMS (Xbridge C18 column, eluting with a gradient of acetonitrile/water containing 0.1% TFA, at flow rate of 60 mL/min). LCMS calculated for C₂₈H₂₇D₃F₃N₆O₄ (M+H)⁺: m/z=574.3; found: 574.3.

### Example 17. Methyl ((1R,3R)-3-(6-((4-(2-methoxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

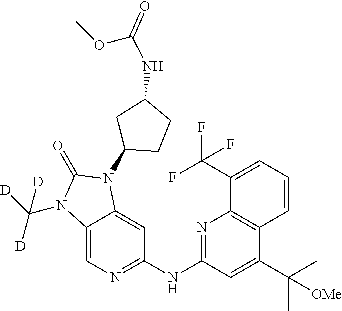 [SMILES: [2H]C([2H])([2H])n1c(=O)n([C@@H]2CC[C@@H](NC(=O)OC)C2)c2cc(Nc3cc(C(C)(C)OC)c4cccc(C(F)(F)F)c4n3)ncc21]

### Step 1. 2-(2-Chloro-8-(trifluoromethyl)quinolin-4-yl)propan-2-ol

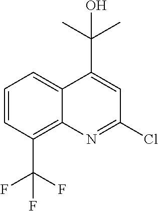 [SMILES: CC(C)(O)c1cc(Cl)nc2c(C(F)(F)F)cccc12]

To a vial containing ethyl 2-chloro-8-(trifluoromethyl)quinoline-4-carboxylate (607 mg, 2 mmol) in THF (10 ml) was added dropwise methylmagnesium bromide (4 ml, 3M in Et₂O) at 0° C. The reaction was stirred at r.t for 15 min. The mixture was quenched with sat. NH₄Cl and extracted with EtOAc. The organic layers were combined, dried over Na₂SO₄, filtered, and the solvents were evaporated in vacuo. The crude material was used in the next step without further purification. LCMS calculated for C₁₃H₁₂ClF₃NO (M+H)⁺: m/z=290.1; found: 290.1.

### Step 2. 2-Chloro-4-(2-methoxypropan-2-yl)-8-(trifluoromethyl)quinoline

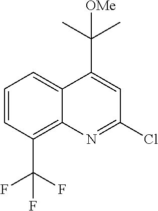 [SMILES: COC(C)(C)c1cc(Cl)nc2c(C(F)(F)F)cccc12]

To a vial containing 2-(2-chloro-8-(trifluoromethyl)quinolin-4-yl)propan-2-ol (100 mg, 0.35 mmol) and iodomethane (147 mg, 1.0 mmol) in THF (3 mL) was added slowly sodium hydride (60% dispersion in mineral oil, 42 mg, 1.0 mmol). After stirring overnight at r.t., the mixture was quenched with sat. NH₄Cl and extracted with EtOAc. The organic layers were combined, dried over Na₂SO₄, filtered, and the solvents were evaporated in vacuo. The crude material was purified by Biotage Isolera. LCMS calculated for C₁₄H₁₄ClF₃NO (M+H)⁺: m/z=304.1; found: 304.1.

### Step 3. Methyl ((1R,3R)-3-(6-((4-(2-methoxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

This compound was prepared according to the procedures described in Example 9, with 2-chloro-4-(2-methoxypropan-2-yl)-8-(trifluoromethyl)quinoline replacing 2-(2-amino-8-(trifluoromethyl)quinolin-4-yl)propan-2-ol. LCMS calculated for C₂₈H₂₉D₃F₃N₆O₄ (M+H)⁺: m/z=576.3; found 576.3. ¹H NMR (400 MHz, DMSO) δ 11.57 (s, 1H), 9.02 (d, J=8.6 Hz, 1H), 8.40 (s, 1H), 8.16 (d, J=7.6 Hz, 1H), 7.80 (s, 1H), 7.61 (t, J=7.8 Hz, 1H), 7.44 (s, 1H), 7.36 (s, 1H), 4.93 (t, J=7.8 Hz, 1H), 4.26 (q, J=6.9 Hz, 1H), 3.55 (s, 3H), 3.01 (s, 3H), 2.40-2.26 (m, 1H), 2.27-2.05 (m, 3H), 2.01-1.87 (m, 1H), 1.72 (s, 6H), 1.70-1.52 (m, 1H).

### Example 18. Methyl ((1R,3R)-3-(6-((4-(cyanomethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

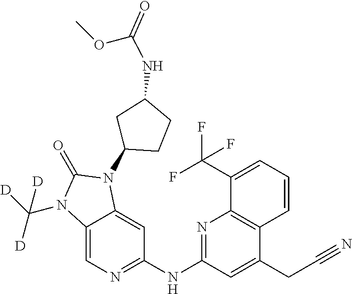 [SMILES: [2H]C([2H])([2H])n1c(=O)n([C@@H]2CC[C@@H](NC(=O)OC)C2)c2cc(Nc3cc(CC#N)c4cccc(C(F)(F)F)c4n3)ncc21]

### Step 1. Methyl ((1R,3R)-3-(6-((4-(hydroxymethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

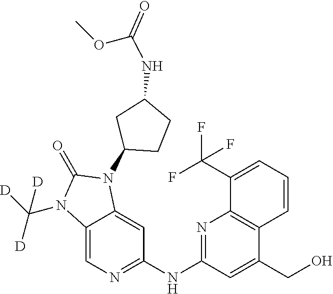 [SMILES: [2H]C([2H])([2H])n1c(=O)n([C@@H]2CC[C@@H](NC(=O)OC)C2)c2cc(Nc3cc(CO)c4cccc(C(F)(F)F)c4n3)ncc21]

To a vial containing ethyl 2-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)-8-(trifluoromethyl)quinoline-4-carboxylate (100 mg, 0.17 mmol) in DCM:MeOH (10 mL, 1:1, v/v) was added NaBH₄ (20 mg, 0.52 mmol). After stirring at r.t for 3 h, the mixture was quenched with sat. NH₄Cl and extracted with EtOAc. The organic layers were combined, dried over Na₂SO₄, filtered, and the solvents were evaporated in vacuo. The crude material was used in the next step without further purification. LCMS calculated for C₂₅H₂₃D₃F₃N₆O₄ (M+H)⁺: m/z=534.2; found: 534.2.

### Step 2. Methyl ((1R,3R)-3-(6-((4-(chloromethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

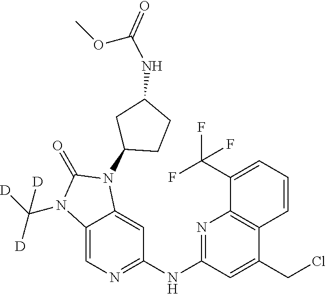 [SMILES: [2H]C([2H])([2H])n1c(=O)n([C@@H]2CC[C@@H](NC(=O)OC)C2)c2cc(Nc3cc(CCl)c4cccc(C(F)(F)F)c4n3)ncc21]

To a vial containing methyl ((1R,3R)-3-(6-((4-(hydroxymethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate (50 mg, 0.094 mmol) in DCM (2 mL) was added SOCl₂ (112 mg, 0.94 mmol) dropwise. After stirring r.t for 1 h, the mixture was quenched with sat. NaHCO₃ and extracted with EA. The organic layers were combined, dried over Na₂SO₄, filtered, and the solvents were evaporated in vacuo. The crude material was used in the next step without further purification. LCMS calculated for C₂₅H₂₂D₃ClF₃N₆O₃ (M+H)⁺: m/z=552.2; found: 552.2.

### Step 3. Methyl ((1R,3R)-3-(6-((4-(cyanomethyl)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

To a vial containing methyl ((1R,3R)-3-(6-((4-(chloromethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate (20 mg, 0.036 mmol) in DMF (0.3 mL) was added KCN (10 mg, 0.15 mmol). The reaction was stirred at 80° C. for 6 h. Upon completion, the reaction was diluted with DCM, filtered through Celite and concentrated in vacuo. The crude material was diluted with acetonitrile and purified by prep-LCMS (Xbridge C18 column, eluting with a gradient of acetonitrile/water containing 0.1% TFA, at flow rate of 60 mL/min). LCMS calculated for C₂₆H₂₂D₃F₃N₇O₃ (M+H)⁺: m/z=543.2; found 543.2. ¹H NMR (400 MHz, DMSO) δ 11.11 (s, 1H), 8.31 (s, 1H), 8.24 (d, J=8.2 Hz, 1H), 8.17 (d, J=7.5 Hz, 1H), 8.02 (s, 1H), 7.63 (t, J=8.0 Hz, 1H), 7.60 (s, 1H), 7.34 (d, J=7.0 Hz, 1H), 5.02-4.82 (m, 1H), 4.68 (s, 2H), 4.26 (q, J=6.9 Hz, 1H), 3.55 (s, 3H), 2.44-2.31 (m, 1H), 2.27-2.04 (m, 3H), 2.02-1.89 (m, 1H), 1.69-1.54 (m, 1H).

### Example 19. Methyl ((1R,3R)-3-(6-((4-(2-cyanopropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

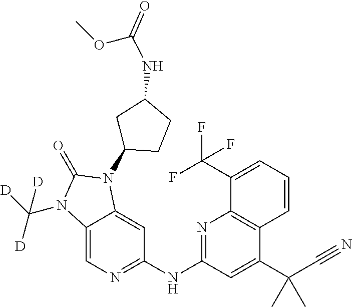 [SMILES: [2H]C([2H])([2H])n1c(=O)n([C@@H]2CC[C@@H](NC(=O)OC)C2)c2cc(Nc3cc(C(C)(C)C#N)c4cccc(C(F)(F)F)c4n3)ncc21]

### Step 1. (2-((2,4-Dimethoxybenzyl)amino)-8-(trifluoromethyl)quinolin-4-yl)methanol

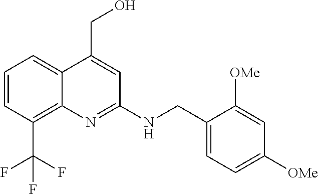 [SMILES: COc1ccc(CNc2cc(CO)c3cccc(C(F)(F)F)c3n2)c(OC)c1]

To a vial containing ethyl 2-((2,4-dimethoxybenzyl)amino)-8-(trifluoromethyl)quinoline-4-carboxylate (1 g, 2.3 mmol) in DCM:MeOH (20 mL, 1:1, v/v) was added NaBH₄ (0.44 g, 11.5 mmol). After stirring at r.t for 3 h, the mixture was quenched with sat. NH₄Cl and extracted with EtOAc. The organic layers were combined, dried over Na₂SO₄, filtered, and the solvents were evaporated in vacuo. The crude material was used in the next step without further purification. LCMS calculated for C₂₀H₂₀F₃N₂O₃(M+H)⁺: m/z=393.2; found: 393.2.

### Step 2. (2-((2,4-Dimethoxybenzyl)amino)-8-(trifluoromethyl)quinolin-4-yl)methyl methanesulfonate

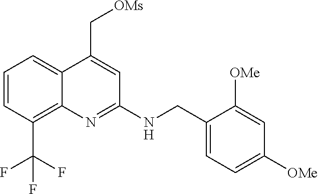 [SMILES: COc1ccc(CNc2cc(COS(C)(=O)=O)c3cccc(C(F)(F)F)c3n2)c(OC)c1]

To a vial containing (2-((2,4-dimethoxybenzyl)amino)-8-(trifluoromethyl)quinolin-4-yl)methanol (200 mg, 0.5 mmol) and triethyl amine (0.17 mL, 1.25 mmol) in DCM (2 mL) was added methanesulfonyl chloride (69 mg, 0.6 mmol) dropwise at 0° C. After stirring r.t for 2 h, the mixture was quenched with water and extracted with EtOAc. The organic layers were combined, dried over Na₂SO₄, filtered, and the solvents were evaporated in vacuo. The crude material was used in the next step without further purification. LCMS calculated for C₂₁H₂₂F₃N₂O₅S (M+H)⁺: m/z=471.1; found: 471.1.

### Step 3. 2-(2-((2,4-Dimethoxybenzyl)amino)-8-(trifluoromethyl)quinolin-4-yl)acetonitrile

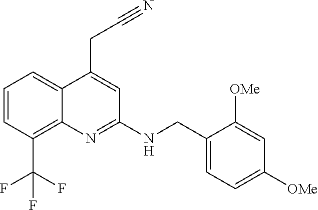 [SMILES: COc1ccc(CNc2cc(CC#N)c3cccc(C(F)(F)F)c3n2)c(OC)c1]

The title compound was prepared according to the procedures described in Example 18, Step 3, with (2-((2,4-dimethoxybenzyl)amino)-8-(trifluoromethyl)quinolin-4-yl)methyl methanesulfonate replacing ((1R,3R)-3-(6-((4-(chloromethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate. LCMS calculated for C₂₁H₁₉F₃N₃O₂(M+H)⁺: m/z=402.2; found 402.2.

### Step 4. Tert-Butyl (4-(cyanomethyl)-8-(trifluoromethyl)quinolin-2-yl)(2,4-dimethoxybenzyl)carbamate

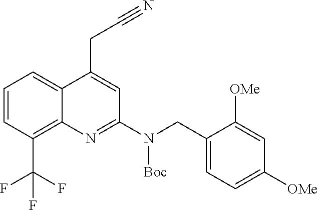 [SMILES: COc1ccc(CN(C(=O)OC(C)(C)C)c2cc(CC#N)c3cccc(C(F)(F)F)c3n2)c(OC)c1]

A solution of 2-(2-((2,4-dimethoxybenzyl)amino)-8-(trifluoromethyl)quinolin-4-yl)acetonitrile (2 g, 5 mmol), di-tert-butyl decarbonate (1.6 g, 7.5 mmol) and N,N-dimethylpyridin-4-amine (0.18 g, 1.5 mmol) in DCM (25 mL) was stirred at r.t for 1 h. The mixture was quenched with sat. NaHCO₃ and extracted with DCM. The organic layers were combined, dried over Na₂SO₄, filtered, and the solvents were evaporated in vacuo. The crude material was used in the next step without further purification. LCMS calculated for C₂₆H₂₇F₃N₃O₄(M+H)⁺: m/z=502.2; found 502.2.

### Step 5. Tert-Butyl (4-(2-cyanopropan-2-yl)-8-(trifluoromethyl)quinolin-2-yl)(2,4-dimethoxybenzyl)carbamate

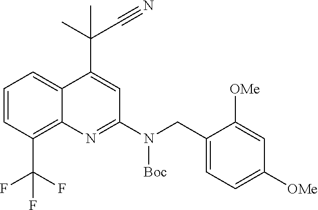 [SMILES: COc1ccc(CN(C(=O)OC(C)(C)C)c2cc(C(C)(C)C#N)c3cccc(C(F)(F)F)c3n2)c(OC)c1]

A mixture of tert-butyl (4-(cyanomethyl)-8-(trifluoromethyl)quinolin-2-yl)(2,4-dimethoxybenzyl)carbamate (150 mg, 0.3 mmol), iodomethane (212 mg, 1.5 mmol), tetrabutylammonium bromide (33 mg, 0.1 mmol) and KOH (10 M in water, 1 mL, 10 mmol) in THF (6 mL) was stirred at 80° C. overnight. The mixture was quenched with sat. NaHCO₃ and extracted with EA. The organic layers were combined, dried over Na₂SO₄, filtered, and the solvents were evaporated in vacuo. The crude material was used in the next step without further purification. LCMS calculated for C₂₈H₃₁F₃N₃O₄(M+H)⁺: m/z=530.2; found 530.2.

### Step 6. 2-(2-Amino-8-(trifluoromethyl)quinolin-4-yl)-2-methylpropanenitrile

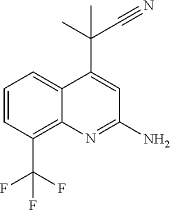 [SMILES: CC(C)(C#N)c1cc(N)nc2c(C(F)(F)F)cccc12]

A solution of tert-butyl (4-(2-cyanopropan-2-yl)-8-(trifluoromethyl)quinolin-2-yl)(2,4-dimethoxybenzyl)carbamate (53 mg, 0.1 mmol) in TFA (5 mL) was stirred at 100° C. for 10 min. The reaction mixture was cooled to r.t, concentrated to remove volatiles, diluted with EtOAc and neutralized with sat. NaHCO₃. The organic layer was subsequently washed with water and brine, dried over Na₂SO₄, filtered, and the solvents were evaporated in vacuo. The crude material was purified by Biotage Isolera. LCMS calculated for C₁₄H₁₃F₃N₃ (M+H)⁺: m/z=280.1; found 280.1.

### Step 7. Methyl ((1R,3R)-3-(6-((4-(2-cyanopropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

The title compound was prepared according to the procedures described in Example 9 with 2-(2-amino-8-(trifluoromethyl)quinolin-4-yl)-2-methylpropanenitrile replacing 2-(2-amino-8-(trifluoromethyl)quinolin-4-yl)propan-2-ol. LCMS calculated for C₂₈H₂₆D₃ClF₃N₇O₃ (M+H)⁺: m/z=571.3; found 571.3. ¹H NMR (400 MHz, DMSO) δ 11.07 (s, 1H), 8.65 (d, J=7.6 Hz, 1H), 8.30 (s, 1H), 8.17 (d, J=7.6 Hz, 1H), 8.08 (s, 1H), 7.68 (t, J=7.9 Hz, 1H), 7.58 (s, 1H), 7.34 (s, 1H), 4.88 (s, 1H), 4.26 (q, J=6.9 Hz, 1H), 3.55 (s, 3H), 2.44-1.97 (m, 5H), 1.95 (s, 6H), 1.60 (d, J=11.7 Hz, 1H).

### Example 20. Methyl ((1R,3R)-3-(6-((6-chloro-4-(2-hydroxypropan-2-yl)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

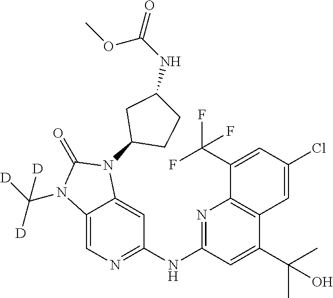 [SMILES: [2H]C([2H])([2H])n1c(=O)n([C@@H]2CC[C@@H](NC(=O)OC)C2)c2cc(Nc3cc(C(C)(C)O)c4cc(Cl)cc(C(F)(F)F)c4n3)ncc21]

### Step 1. Ethyl 2,6-dichloro-8-(trifluoromethyl)quinoline-4-carboxylate

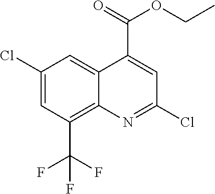 [SMILES: CCOC(=O)c1cc(Cl)nc2c(C(F)(F)F)cc(Cl)cc12]

This compound was prepared according to the procedures described in Intermediate 2, Step 1-3, with 5-chloro-7-(trifluoromethyl)indoline-2,3-dione replacing 7-(trifluoromethyl)indoline-2,3-dione in Step 1. LCMS calculated for C₁₃H₉Cl₂F₃NO₂ (M+H)⁺: m/z=338.0; found 338.0.

### Step 2. 2-(2,6-Dichloro-8-(trifluoromethyl)quinolin-4-yl)propan-2-ol

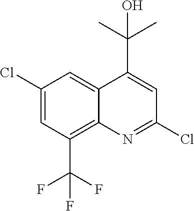 [SMILES: CC(C)(O)c1cc(Cl)nc2c(C(F)(F)F)cc(Cl)cc12]

To a solution of ethyl 2,6-dichloro-8-(trifluoromethyl)quinoline-4-carboxylate (0.34 g, 1.0 mmol) in THF (10 ml) was added dropwise methylmagnesium bromide (2 ml, 3M in Et₂O) at 0° C. The reaction was stirred at r.t for 15 min. The mixture was quenched with sat. NH₄Cl and extracted with EtOAc. The organic layer was combined, dried over Na₂SO₄, filtered, and the solvents were evaporated in vacuo. The crude material was purified by Biotage Isolera to give the product as a light-yellow solid. LCMS calculated for C₁₃H₁₁Cl₂F₃NO (M+H)⁺: m/z=324.0; found 324.0.

### Step 3. Methyl ((1R,3R)-3-(6-((tert-butoxycarbonyl)amino)-3-(methyl-d₃)-2-oxo-2,3-dihydro-1H-imidazo[4,5-c]pyridin-1-yl)cyclopentyl)carbamate

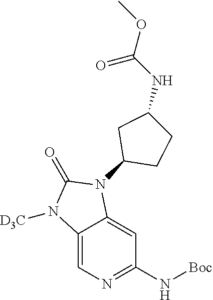 [SMILES: [2H]C([2H])([2H])n1c(=O)n([C@@H]2CC[C@@H](NC(=O)OC)C2)c2cc(NC(=O)OC(C)(C)C)ncc21]

A reaction vial containing methyl ((1R,3R)-3-(6-chloro-3-(methyl-d₃)-2-oxo-2,3-dihydro-1H-imidazo[4,5-c]pyridin-1-yl)cyclopentyl)carbamate (1 g, 3.0 mmol), tert-butyl carbamate (1.4 g, 12.2 mmol), xantphos (0.49 g, 0.92 mmol), Pd₂(dba)₃ (0.42 g, 0.46 mmol) and cesium carbonate (2.5 g, 7.6 mmol) was evacuated and backfilled with nitrogen. 1,4-Dioxane (20 mL) was added to the reaction mixture, which was then stirred at 97° C. for 24 h. Upon completion, the reaction was diluted with DCM, filtered through Celite and concentrated in vacuo. The crude material was purified by Biotage Isolera to give the product as a light-yellow solid. LCMS calculated for C₁₉H₂₅D₃N₅O₅(M+H)⁺: m/z=409.3; found 409.3.

### Step 4. Methyl ((1R,3R)-3-(6-amino-3-(methyl-d₃)-2-oxo-2,3-dihydro-1H-imidazo[4,5-c]pyridin-1-yl)cyclopentyl)carbamate

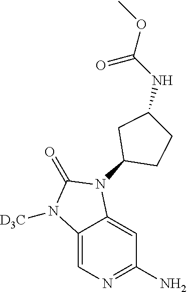 [SMILES: [2H]C([2H])([2H])n1c(=O)n([C@@H]2CC[C@@H](NC(=O)OC)C2)c2cc(N)ncc21]

To a reaction vial containing methyl ((1R,3R)-3-(6-((tert-butoxycarbonyl)amino)-3-(methyl-d₃)-2-oxo-2,3-dihydro-1H-imidazo[4,5-c]pyridin-1-yl)cyclopentyl)carbamate (1.2 g, 3.0 mmol) was added trifluoroacetic acid (5 mL). The reaction mixture was then stirred at r.t for 1 h. The reaction mixture was concentrated in vacuo and the crude material was used directly for next step. LCMS calculated for C₁₄H₁₇D₃N₅O₃(M+H)⁺: m/z=309.2; found 309.2.

### Step 5. Methyl ((1R,3R)-3-(6-((6-chloro-4-(2-hydroxypropan-2-yl)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

This compound was prepared according to the procedures described in Example 9 with methyl ((1R,3R)-3-(6-amino-3-(methyl-d₃)-2-oxo-2,3-dihydro-1H-imidazo[4,5-c]pyridin-1-yl)cyclopentyl)carbamate replacing methyl ((1R,3R)-3-(6-chloro-3-(methyl-d₃)-2-oxo-2,3-dihydro-1H-imidazo[4,5-c]pyridin-1-yl)cyclopentyl)carbamate and 2-(2,6-dichloro-8-(trifluoromethyl)quinolin-4-yl)propan-2-ol replacing 2-(2-amino-8-(trifluoromethyl)quinolin-4-yl)propan-2-ol. LCMS calculated for C₂₇H₂₆D₃ClF₃N₆O₄ (M+H)⁺: m/z=596.2; found 596.2. ¹H NMR (400 MHz, DMSO) δ 10.73 (s, 1H), 9.09 (d, J=2.4 Hz, 1H), 8.26 (s, 1H), 8.14 (s, 1H), 8.08 (s, 1H), 7.54 (s, 1H), 7.33 (d, J=6.7 Hz, 1H), 5.77 (s, 1H), 4.99-4.75 (m, 1H), 4.26 (q, J=6.9 Hz, 1H), 3.55 (s, 3H), 2.45-2.31 (m, 1H), 2.26-2.16 (m, 1H), 2.16-2.06 (m, 2H), 2.02-1.88 (m, 1H), 1.68 (s, 6H), 1.65-1.52 (m, 1H).

### Example 21. Methyl ((1R,3R)-3-(6-((4-chloro-8-(trifluoromethyl)quinolin-2-yl)amino)-3-methyl-2-oxo-2,3-dihydro-1H-imidazo[4,5-c]pyridin-1-yl)cyclopentyl)carbamate

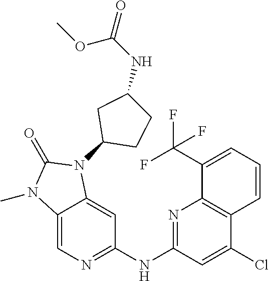 [SMILES: COC(=O)N[C@@H]1CC[C@@H](n2c(=O)n(C)c3cnc(Nc4cc(Cl)c5cccc(C(F)(F)F)c5n4)cc32)C1]

### Step 1. Methyl ((1R,3R)-3-(6-chloro-3-methyl-2-oxo-2,3-dihydro-1H-imidazo[4,5-c]pyridin-1-yl)cyclopentyl)carbamate

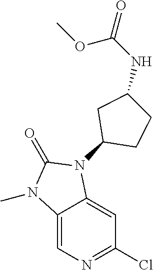 [SMILES: COC(=O)N[C@@H]1CC[C@@H](n2c(=O)n(C)c3cnc(Cl)cc32)C1]

The title compound was prepared according to the procedures described in Intermediate 1, Step 1-5, with iodomethane replacing iodomethane-d₃ in Step 4. LCMS calculated for C₁₄H₁₈ClN₄O₃(M+H)⁺: m/z=325.1; found 325.1.

### Step 2. Methyl ((1R,3R)-3-(6-((4-chloro-8-(trifluoromethyl)quinolin-2-yl)amino)-3-methyl-2-oxo-2,3-dihydro-1H-imidazo[4,5-c]pyridin-1-yl)cyclopentyl)carbamate

The title compound was prepared according to the procedures described in Example 9, with methyl ((1R,3R)-3-(6-chloro-3-methyl-2-oxo-2,3-dihydro-1H-imidazo[4,5-c]pyridin-1-yl)cyclopentyl)carbamate replacing methyl ((1R,3R)-3-(6-chloro-3-(methyl-d₃)-2-oxo-2,3-dihydro-1H-imidazo[4,5-c]pyridin-1-yl)cyclopentyl)carbamate and 4-chloro-8-(trifluoromethyl)quinolin-2-amine replacing 2-(2-amino-8-(trifluoromethyl)quinolin-4-yl)propan-2-ol. LCMS calculated for C₂₄H₂₃ClF₃N₆O₃ (M+H)⁺: m/z=535.2; found 535.2. ¹H NMR (400 MHz, DMSO) δ 10.48 (s, 1H), 8.38 (s, 1H), 8.33 (d, J=8.3 Hz, 1H), 8.18 (s, 1H), 8.16 (d, J=7.7 Hz, 1H), 7.72 (s, 1H), 7.60 (t, J=7.8 Hz, 1H), 7.31 (d, J=7.1 Hz, 1H), 4.94-4.75 (m, 1H), 4.26 (q, J=6.9 Hz, 1H), 3.54 (s, 3H), 3.37 (s, 3H), 2.46-2.29 (m, 1H), 2.27-2.15 (m, 1H), 2.16-2.05 (m, 2H), 2.02-1.85 (m, 1H), 1.79-1.41 (m, 1H).

### Example 22. Methyl ((1R,3R)-3-(6-((4-(2-methoxyacetamido)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

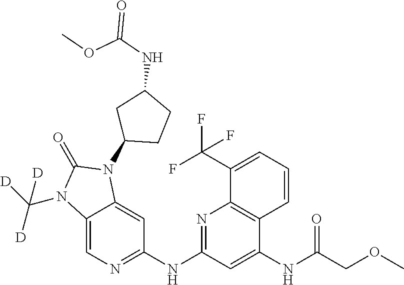 [SMILES: [2H]C([2H])([2H])n1c(=O)n([C@@H]2CC[C@@H](NC(=O)OC)C2)c2cc(Nc3cc(NC(=O)COC)c4cccc(C(F)(F)F)c4n3)ncc21]

### Step 1. 2-Chloro-8-(trifluoromethyl)quinolin-4-amine

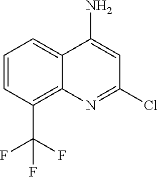 [SMILES: Nc1cc(Cl)nc2c(C(F)(F)F)cccc12]

This compound was prepared according to the procedures described in Intermediate 3, Step 1-4. 2-Chloro-8-(trifluoromethyl)quinolin-4-amine was eluted in 25% EA/hex. LCMS calculated for C₁₀H₇ClF₃N₂(M+H)⁺: m/z=247.0; found 247.0.

### Step 2. N-(2-Chloro-8-(trifluoromethyl)quinolin-4-yl)-2-methoxyacetamide

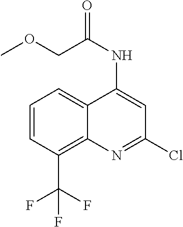 [SMILES: COCC(=O)Nc1cc(Cl)nc2c(C(F)(F)F)cccc12]

To a solution of 2-chloro-8-(trifluoromethyl)quinolin-4-amine (50 mg, 0.2 mmol) in pyridine (0.2 mL) was added 2-methoxyacetyl chloride (44 mg, 0.4 mmol). The mixture was stirred at r.t for 1 h. Water was added, the precipitated solid was collected by filtration and used in the next step without further purification. LCMS calculated for C₁₃H₁₁ClF₃N₂O₂ (M+H)⁺: m/z=319.1; found 319.1.

### Step 3. Methyl ((1R,3R)-3-(6-((4-(2-methoxyacetamido)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

This compound was prepared according to the procedures described in Example 9, with N-(2-chloro-8-(trifluoromethyl)quinolin-4-yl)-2-methoxyacetamide replacing 2-(2-amino-8-(trifluoromethyl)quinolin-4-yl)propan-2-ol. LCMS calculated for C₂₇H₂₆D₃F₃N₇O₅ (M+H)⁺: m/z=591.3; found 591.3. ¹H NMR (400 MHz, DMSO) δ 11.55 (s, 1H), 10.32 (s, 1H), 8.49 (d, J=8.5 Hz, 1H), 8.40 (s, 1H), 8.21 (d, J=7.5 Hz, 1H), 8.13 (s, 1H), 7.66 (t, J=8.0 Hz, 1H), 7.58 (s, 1H), 7.37 (d, J=7.0 Hz, 1H), 4.94 (p, J=8.6 Hz, 1H), 4.30 (s, 2H), 4.25 (q, J=6.7 Hz, 1H), 3.56 (s, 3H), 3.47 (s, 3H), 2.34 (m, 1H), 2.25-1.88 (m, 4H), 1.74-1.55 (m, 1H).

### Example 23. Methyl ((1R,3R)-3-(3-(methyl-d₃)-6-((4-(methylsulfonamido)-8-(trifluoromethyl)quinolin-2-yl)amino)-2-oxo-2,3-dihydro-1H-imidazo[4,5-c]pyridin-1-yl)cyclopentyl)carbamate

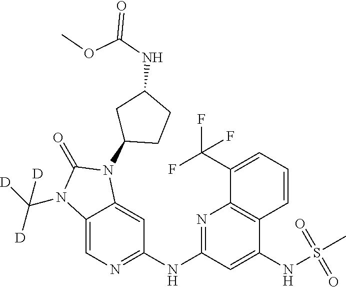 [SMILES: [2H]C([2H])([2H])n1c(=O)n([C@@H]2CC[C@@H](NC(=O)OC)C2)c2cc(Nc3cc(NS(C)(=O)=O)c4cccc(C(F)(F)F)c4n3)ncc21]

### Step 1. N-(2-Chloro-8-(trifluoromethyl)quinolin-4-yl)methanesulfonamide

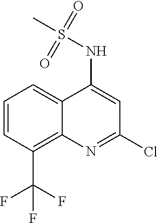 [SMILES: CS(=O)(=O)Nc1cc(Cl)nc2c(C(F)(F)F)cccc12]

The title compound was prepared according to the procedures described in Example 22, Step 2, with methanesulfonyl chloride replacing 2-methoxyacetyl chloride. LCMS calculated for C₁₁H₉ClF₃N₂O₂S (M+H)⁺: m/z=325.0; found 325.0.

### Step 2. Methyl ((1R,3R)-3-(3-(methyl-d₃)-6-((4-(methylsulfonamido)-8-(trifluoromethyl)quinolin-2-yl)amino)-2-oxo-2,3-dihydro-1H-imidazo[4,5-c]pyridin-1-yl)cyclopentyl)carbamate

The title compound was prepared according to the procedures described in Example 9, with N-(2-chloro-8-(trifluoromethyl)quinolin-4-yl)methanesulfonamide replacing 2-(2-amino-8-(trifluoromethyl)quinolin-4-yl)propan-2-ol. LCMS calculated for C₂₅H₂₄D₃F₃N₇O₅S (M+H)⁺: m/z=597.2; found 597.2. ¹H NMR (400 MHz, DMSO) δ 11.25 (s, 1H), 8.58 (d, J=8.3 Hz, 1H), 8.25 (s, 1H), 8.15 (d, J=7.5 Hz, 1H), 7.57 (t, J=7.9 Hz, 1H), 7.38 (d, J=7.2 Hz, 1H), 7.21 (s, 1H), 7.09 (s, 1H), 5.02-4.86 (m, 1H), 4.24 (q, J=6.7 Hz, 1H), 3.56 (s, 3H), 3.47 (s, 1H), 3.13 (s, 3H), 2.37-1.82 (m, 5H), 1.65 (dd, J=12.9, 7.2 Hz, 1H).

### Example 24. Methyl ((1R,3R)-3-(6-((8-bromoquinolin-2-yl)amino)-3-methyl-2-oxo-2,3-dihydro-1H-imidazo[4,5-c]pyridin-1-yl)cyclopentyl)carbamate

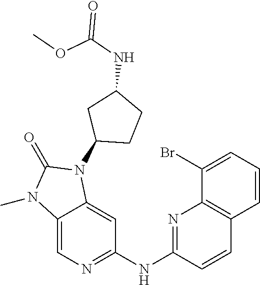 [SMILES: COC(=O)N[C@@H]1CC[C@@H](n2c(=O)n(C)c3cnc(Nc4ccc5cccc(Br)c5n4)cc32)C1]

The title compound was prepared according to the procedures described in Example 9, with methyl ((1R,3R)-3-(6-chloro-3-methyl-2-oxo-2,3-dihydro-1H-imidazo[4,5-c]pyridin-1-yl)cyclopentyl)carbamate replacing methyl ((1R,3R)-3-(6-chloro-3-(methyl-d₃)-2-oxo-2,3-dihydro-1H-imidazo[4,5-c]pyridin-1-yl)cyclopentyl)carbamate and 8-bromoquinolin-2-amine replacing 2-(2-amino-8-(trifluoromethyl)quinolin-4-yl)propan-2-ol. LCMS calculated for C₂₃H₂₄BrN₆O₃(M+H)⁺: m/z=511.1; found 511.1. ¹H NMR (400 MHz, DMSO) δ 11.76 (s, 1H), 8.52 (s, 1H), 8.43 (s, 1H), 8.14 (d, J=7.6 Hz, 1H), 7.98 (d, J=8.1 Hz, 1H), 7.56-7.31 (m, 3H), 6.52 (s, 1H), 4.96 (q, J=8.6 Hz, 1H), 4.23 (q, J=6.8 Hz, 1H), 3.56 (s, 3H), 3.40 (s, 3H), 2.42-2.28 (m, 1H), 2.27-1.90 (m, 4H), 1.74-1.56 (m, 1H).

### Example 25. Methyl ((1R,3R)-3-(6-((6-chloro-8-(trifluoromethyl)quinolin-2-yl)amino)-3-methyl-2-oxo-2,3-dihydro-1H-imidazo[4,5-c]pyridin-1-yl)cyclopentyl)carbamate

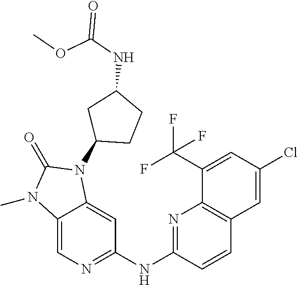 [SMILES: COC(=O)N[C@@H]1CC[C@@H](n2c(=O)n(C)c3cnc(Nc4ccc5cc(Cl)cc(C(F)(F)F)c5n4)cc32)C1]

### Step 1. Ethyl 3-(2-amino-5-chloro-3-(trifluoromethyl)phenyl)acrylate

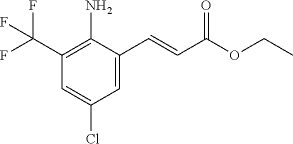 [SMILES: CCOC(=O)/C=C/c1cc(Cl)cc(C(F)(F)F)c1N]

A vial containing 2-bromo-4-chloro-6-(trifluoromethyl)aniline (933 mg, 3.4 mmol), ethyl acrylate (0.724 mL, 6.8 mmol), DIPEA (1.8 mL, 10.2 mmol), tri-o-tolylphosphane (207 mg, 0.68 mmol) and Pd(OAc)₂ (152 mg, 0.68 mmol) was evacuated and backfilled with nitrogen. DMF (7 mL) was added to the reaction mixture, which was then stirred at 100° C. overnight. The reaction was quenched by the addition of water and extracted with EtOAc. The organic layer was dried over Na₂SO₄, filtered, and the solvents were evaporated in vacuo. The crude material was purified by Biotage Isolera. LCMS calculated for C₁₂H₁₂ClF₃NO₂ (M+H)⁺: m/z=294.0; found 294.0.

### Step 2. 6-Chloro-8-(trifluoromethyl)quinolin-2(1H)-one

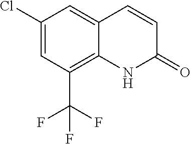 [SMILES: O=c1ccc2cc(Cl)cc(C(F)(F)F)c2[nH]1]

To a suspension of ethyl 3-(2-amino-5-chloro-3-(trifluoromethyl)phenyl)acrylate (587 mg, 2 mmol) in 1,4-dioxane (4 mL) was added 12 M HCl (3 mL). The mixture was stirred at 100° C. for 5 h. The reaction mixture was cooled to r.t and quenched by the addition of water. The solid was collected by filtration and used in the next step without further purification. LCMS calculated for C₁₀H₆ClF₃NO (M+H)⁺: m/z=248.0; found 248.0.

### Step 3. 2,6-Dichloro-8-(trifluoromethyl)quinoline

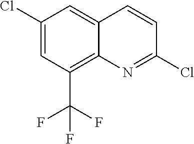 [SMILES: FC(F)(F)c1cc(Cl)cc2ccc(Cl)nc12]

A mixture of 6-chloro-8-(trifluoromethyl)quinolin-2(1H)-one (495 mg, 2 mmol) and phosphoryl trichloride (5 mL) was stirred at 110° C. for 1 h. The reaction mixture was allowed to cool to r.t and concentrated in vacuo. The residue was quenched with ice water. The solid was collected by filtration and used in the next step without further purification. LCMS calculated for C₁₀H₅Cl₂F₃N (M+H)⁺: m/z=266.0; found 266.0.

### Step 4. 6-Chloro-8-(trifluoromethyl)quinolin-2-amine

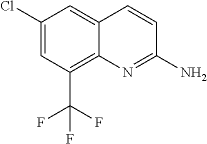 [SMILES: Nc1ccc2cc(Cl)cc(C(F)(F)F)c2n1]

To a vial containing 2,6-dichloro-8-(trifluoromethyl)quinoline (400 mg, 1.5 mmol) and 1,4-dioxane (4 mL) was added ammonia hydroxide (4 mL), which was then microwaved at 130° C. for 2 h. The reaction mixture was cooled to r.t and extracted with EtOAc. The organic layer was dried over Na₂SO₄, filtered, and the solvents were evaporated in vacuo. The crude material was used in the next step without further purification. LCMS calculated for C₁₀H₇ClF₃N₂(M+H)⁺: m/z=247.0; found 247.0.

### Step 5. Methyl ((1R,3R)-3-(6-((6-chloro-8-(trifluoromethyl)quinolin-2-yl)amino)-3-methyl-2-oxo-2,3-dihydro-1H-imidazo[4,5-c]pyridin-1-yl)cyclopentyl)carbamate

The title compound was prepared according to the procedures described in Example 9, with 6-chloro-8-(trifluoromethyl)quinolin-2-amine replacing 2-(2-amino-8-(trifluoromethyl)quinolin-4-yl)propan-2-ol. LCMS calculated for C₂₄H₂₃ClF₃N₆O₃ (M+H)⁺: m/z=535.2; found 535.2. ¹H NMR (400 MHz, DMSO) δ 10.97 (s, 1H), 8.31 (d, J=9.2 Hz, 1H), 8.30 (s, 1H), 8.28 (s, 1H), 8.10 (s, 1H), 8.09 (s, 1H), 7.49 (d, J=9.1 Hz, 1H), 7.33 (d, J=7.0 Hz, 1H), 4.86 (p, J=8.3 Hz, 1H), 4.26 (m, 1H), 3.55 (s, 3H), 3.39 (s, 3H), 2.38 (m, 1H), 2.28-2.04 (m, 3H), 1.94 (m, 1H), 1.61 (m, 1H).

### Example 26. Methyl ((1R,3R)-3-(6-((6-cyano-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

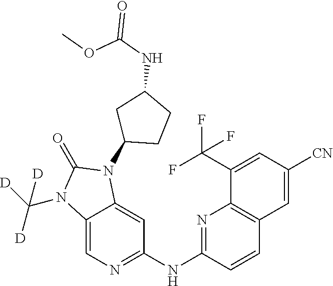 [SMILES: [2H]C([2H])([2H])n1c(=O)n([C@@H]2CC[C@@H](NC(=O)OC)C2)c2cc(Nc3ccc4cc(C#N)cc(C(F)(F)F)c4n3)ncc21]

### Step 1. 2-Amino-8-(trifluoromethyl)quinoline-6-carbonitrile

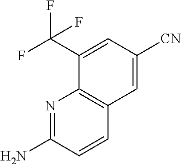 [SMILES: N#Cc1cc(C(F)(F)F)c2nc(N)ccc2c1]

A suspension of 6-chloro-8-(trifluoromethyl)quinolin-2-amine (100 mg, 0.41 mmol), Pd₂(dba)₃ (93 mg, 0.1 mmol), Sphos (83 mg, 0.2 mmol), Zn(CN)₂ (95 mg, 0.8 mmol) in DMF:H₂O (5 ml, 99:1, v/v) was microwaved at 170° C. for 1 h. The solution was diluted with water and extracted with EtOAc. The organic layer was dried over Na₂SO₄, filtered, and the solvents were evaporated in vacuo. The crude material was purified by Biotage Isolera. LCMS calculated for C₁₁H₇F₃O₃(M+H)⁺: m/z=238.1; found 238.1.

### Step 2. Methyl ((1R,3R)-3-(6-((6-cyano-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

The title compound was prepared according to the procedures described in Example 25, Step 5 with 2-amino-8-(trifluoromethyl)quinoline-6-carbonitrile replacing 6-chloro-8-(trifluoromethyl)quinolin-2-amine. LCMS calculated for C₂₅H₂₀D₃F₃N₇O₃ (M+H)⁺: m/z=529.2; found: 529.2. ¹H NMR (400 MHz, DMSO) δ 11.00 (s, 1H), 8.71 (d, J=1.8 Hz, 1H), 8.43 (s, 1H), 8.35 (d, J=9.2 Hz, 1H), 8.28 (s, 1H), 8.24 (s, 1H), 8.16 (d, J=1.9 Hz, 1H), 8.05 (d, J=9.1 Hz, 1H), 4.84 (p, J=9.5 Hz, 1H), 4.25 (p, J=7.0 Hz, 1H), 3.55 (s, 3H), 2.45-1.52 (m, 6H).

### Example 27. Methyl ((1R,3R)-3-(6-((6-fluoro-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

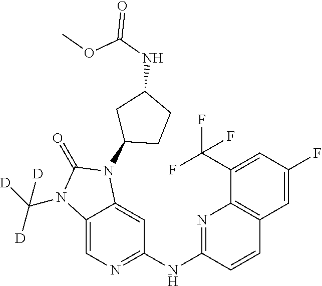 [SMILES: [2H]C([2H])([2H])n1c(=O)n([C@@H]2CC[C@@H](NC(=O)OC)C2)c2cc(Nc3ccc4cc(F)cc(C(F)(F)F)c4n3)ncc21]

The title compound was prepared according to the procedures described in Example 25, Step 1-5 with 6-fluoro-8-(trifluoromethyl)quinolin-2-amine replacing 6-chloro-8-(trifluoromethyl)quinolin-2-amine. LCMS calculated for C₂₄H₂₀D₃F₄N₆O₃ (M+H)⁺: m/z=522.2; found: 522.2. ¹H NMR (400 MHz, DMSO) δ 10.97 (s, 1H), 8.34 (d, J=9.8 Hz, 1H), 8.29 (s, 1H), 8.19-7.97 (m, 3H), 7.50 (d, J=9.1 Hz, 1H), 7.34 (d, J=6.7 Hz, 1H), 4.86 (t, J=8.6 Hz, 1H), 4.26 (q, J=6.9 Hz, 1H), 3.55 (s, 3H), 2.38 (m, 1H), 2.30-2.01 (m, 3H), 2.00-1.88 (m, 1H), 1.61 (m, 1H).

### Example 28. Methyl ((1R,3R)-3-(3-(methyl-d₃)-6-((8-methyl-1,6-naphthyridin-2-yl)amino)-2-oxo-2,3-dihydro-1H-imidazo[4,5-c]pyridin-1-yl)cyclopentyl)carbamate

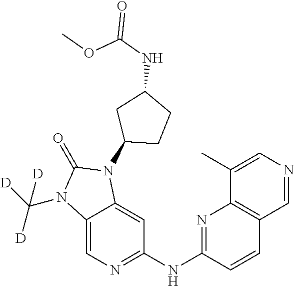 [SMILES: [2H]C([2H])([2H])n1c(=O)n([C@@H]2CC[C@@H](NC(=O)OC)C2)c2cc(Nc3ccc4cncc(C)c4n3)ncc21]

### Step 1. 8-Methyl-1,6-naphthyridin-2-ol

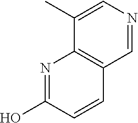

A vial containing 8-bromo-1,6-naphthyridin-2-ol (224 mg, 1.0 mmol), sodium carbonate (318 mg, 3.0 mmol), methylboronic acid (300 mg, 5.0 mmol) and Pd (PPh₃)₄ (170 mg, 0.15 mmol) was evacuated and backfilled with nitrogen. Toluene:water:ethanol (5 mL, 5:5:1, v/v) was added to the reaction mixture, which was then stirred at 100° C. for 6 h. The solution was diluted with water and extracted with EtOAc. The organic layer was dried over Na₂SO₄, filtered, and the solvents were evaporated in vacuo. The crude material was purified by Biotage Isolera. LCMS calculated for C₉H₉N₂O (M+H)⁺: m/z=161.1; found 161.1.

### Step 2. 2-Chloro-8-methyl-4,6-naphthyridine

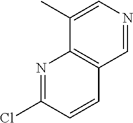

A mixture of 8-methyl-1,6-naphthyridin-2-ol (160 mg, 1.0 mmol) and phosphoryl trichloride (5 mL) was stirred at 110° C. for 1 h. The reaction mixture was allowed to cool to r.t and concentrated in vacuo. The residue was quenched with ice water. The solid was collected by filtration and used in the next step without further purification. LCMS calculated for C₉H₈N₂Cl (M+H)⁺: m/z=179.1; found 179.1.

### Step 3. 8-Methyl-4,6-naphthyridin-2-amine

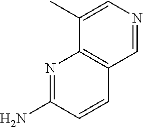

A solution of 2-chloro-8-methyl-1,6-naphthyridine (179 mg, 1.0 mmol) and ammonia hydroxide (4 mL) in 1,4-dioxane (3 mL) was microwaved at 140° C. for 1 h. The reaction mixture was cooled to r.t and extracted with EtOAc. The organic layer was dried over Na₂SO₄, filtered, and the solvents were evaporated in vacuo. The crude material was used in the next step without further purification. LCMS calculated for C₉H₁₀N₃ (M+H)⁺: m/z=160.1; found 160.1.

### Step 4. Methyl ((1R,3R)-3-(3-(methyl-d₃)-6-((8-methyl-1,6-naphthyridin-2-yl)amino)-2-oxo-2,3-dihydro-1H-imidazo[4,5-c]pyridin-1-yl)cyclopentyl)carbamate

The title compound was prepared according to the procedures described in Example 9, with 8-methyl-1,6-naphthyridin-2-amine replacing 2-(2-amino-8-(trifluoromethyl)quinolin-4-yl)propan-2-ol. LCMS calculated for C₂₃H₂₃D₃N₇O₃(M+H)⁺: m/z=451.3; found: 451.3.

### Example 29. Methyl ((1R,3R)-3-(6-((4-(2-hydroxypropan-2-yl)-8-methyl-1,5-naphthyridin-2-yl)amino)-3-(methyl-d₃)-2-oxo-2,3-dihydro-1H-imidazo[4,5-c]pyridin-1-yl)cyclopentyl)carbamate

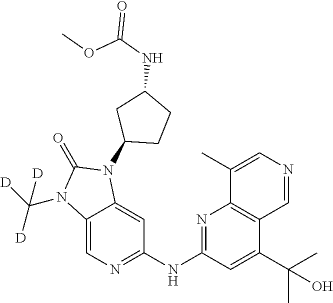 [SMILES: [2H]C([2H])([2H])n1c(=O)n([C@@H]2CC[C@@H](NC(=O)OC)C2)c2cc(Nc3cc(C(C)(C)O)c4cncc(C)c4n3)ncc21]

### Step 1. Methyl 2-chloro-5-(((2,2-dimethyl-4,6-dioxo-1,3-dioxan-5-ylidene)methyl)amino)isonicotinate

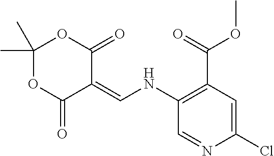 [SMILES: COC(=O)c1cc(Cl)ncc1NC=C1C(=O)OC(C)(C)OC1=O]

A suspension of methyl 5-amino-2-chloroisonicotinate (5 g, 26.8 mmol) and 5-(methoxymethylene)-2,2-dimethyl-1,3-dioxane-4,6-dione (6 g, 32.2 mmol) in toluene (40 mL) was stirred at 110° C. for 2 h. The solution was cooled down and hexane (100 mL) was added. The precipitated solid was collected by filtration and used in the next step without further purification. LCMS calculated for C₁₄H₁₄ClN₂O₆(M+H)⁺: m/z=341.1; found 341.1.

### Step 2. Methyl 2-chloro-8-hydroxy-1,5-naphthyridine-4-carboxylate

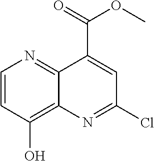 [SMILES: COC(=O)c1cc(Cl)nc2c(O)ccnc12]

A solution of methyl 2-chloro-5-(((2,2-dimethyl-4,6-dioxo-1,3-dioxan-5-ylidene)methyl)amino)isonicotinate (5 g, 14.7 mmol) in Downtherm A (20 mL) was stirred at 250° C. for 10 min. The solution was cooled down and Et₂O (100 mL) was added. The precipitated solid was collected by filtration and used in the next step without further purification. LCMS calculated for C₁₀H₈ClN₂O₃(M+H)⁺: m/z=239.0; found 239.0.

### Step 3. Methyl 8-bromo-2-chloro-1,5-naphthyridine-4-carboxylate

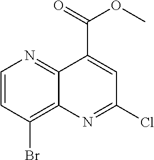 [SMILES: COC(=O)c1cc(Cl)nc2c(Br)ccnc12]

To a vial containing methyl 2-chloro-8-hydroxy-1,5-naphthyridine-4-carboxylate (1.5 g, 6.3 mmol) in DMF (10 mL) was added tribromophosphane (5 mL) at 0° C. After stirring at r.t for 2 h, the reaction mixture was poured into crushed ice and neutralized using sat. NaHCO₃. The suspension was extracted with EtOAc. The organic layer was dried over Na₂SO₄, filtered, and the solvents were evaporated in vacuo. The crude material was purified by Biotage Isolera. LCMS calculated for C₁₀H₇BrClN₂O₂(M+H)⁺: m/z=301.0; found 301.0.

### Step 4. Methyl 8-bromo-2-((2,4-dimethoxybenzyl)amino)-1,5-naphthyridine-4-carboxylate

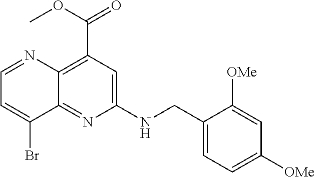 [SMILES: COC(=O)c1cc(NCc2ccc(OC)cc2OC)nc2c(Br)ccnc12]

To a vial containing methyl 8-bromo-2-chloro-1,5-naphthyridine-4-carboxylate (580 mg, 1.9 mmol) and (2,4-dimethoxyphenyl)methanamine (643 mg, 3.9 mmol) in DMSO (5 mL) was added triethylamine (0.67 mL, 4.8 mmol), which was then stirred at 110° C. for 1 h. The reaction mixture was cooled to r.t and water was added. The solid was then filtered and washed with water. The crude material was used in the next step without further purification. LCMS calculated for Cl₉H₁₉BrN₃O₄(M+H)⁺: m/z=432.1; found 432.1.

### Step 5. Methyl 2-((2,4-dimethoxybenzyl)amino)-8-methyl-1,5-naphthyridine-4-carboxylate

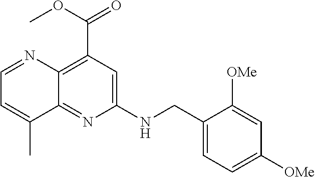 [SMILES: COC(=O)c1cc(NCc2ccc(OC)cc2OC)nc2c(C)ccnc12]

A vial containing methyl 8-bromo-2-((2,4-dimethoxybenzyl)amino)-1,5-naphthyridine-4-carboxylate (310 mg, 0.7 mmol), saturated potassium carbonate solution (1 mL), methylboronic acid (300 mg, 5.0 mmol) and Pd (PPh₃)₄ (170 mg, 0.15 mmol) was evacuated and backfilled with nitrogen. 1,4-Dioxane (4 mL) was added to the reaction mixture, which was then stirred at 100° C. for 6 h. The solution was diluted with water and extracted with EtOAc. The organic layer was dried over Na₂SO₄, filtered, and the solvents were evaporated in vacuo. The crude material was purified by Biotage Isolera. LCMS calculated for C₂₀H₂₂N₃O₄ (M+H)⁺: m/z=368.2; found 368.2.

### Step 6. Methyl 2-amino-8-methyl-1,5-naphthyridine-4-carboxylate

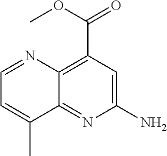 [SMILES: COC(=O)c1cc(N)nc2c(C)ccnc12]

A solution of methyl 2-((2,4-dimethoxybenzyl)amino)-8-methyl-1,5-naphthyridine-4-carboxylate (150 mg, 0.4 mmol) in TFA (3 mL) was stirred at 110° C. for 10 min. The reaction mixture was cooled to r.t, concentrated to remove volatiles, diluted with EtOAc and neutralized with sat. NaHCO₃. The organic layer was subsequently washed with water and brine, dried over Na₂SO₄, filtered, and the solvents were evaporated in vacuo. The crude material was purified by Biotage Isolera. LCMS calculated for C₁₁H₁₂N₃O₂ (M+H)⁺: m/z=218.1; found 218.1.

### Step 7. Methyl 2-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)-8-methyl-1,5-naphthyridine-4-carboxylate

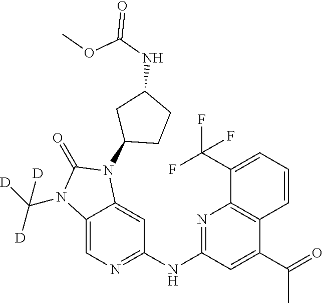 [SMILES: [2H]C([2H])([2H])n1c(=O)n([C@@H]2CC[C@@H](NC(=O)OC)C2)c2cc(Nc3cc(C(C)=O)c4cccc(C(F)(F)F)c4n3)ncc21]

This compound was prepared according to the procedures described in Example 10, Step 1, with methyl 2-amino-8-methyl-1,5-naphthyridine-4-carboxylate replacing ethyl 2-amino-8-(trifluoromethyl)quinoline-4-carboxylate. LCMS calculated for C₂₅H₂₅D₃N₇O₅(M+H)⁺: m/z=509.3; found 509.3.

### Step 8. Methyl ((1R,3R)-3-(6-((4-(2-hydroxypropan-2-yl)-8-methyl-1,5-naphthyridin-2-yl)amino)-3-(methyl-d₃)-2-oxo-2,3-dihydro-1H-imidazo[4,5-c]pyridin-1-yl)cyclopentyl)carbamate

To a vial containing methyl 2-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)-8-methyl-1,5-naphthyridine-4-carboxylate (24 mg, 0.05 mmol) in THF (1 ml) was added dropwise methylmagnesium bromide (0.1 ml, 3M in Et₂O) at 0° C. The reaction was stirred at r.t for 15 min. The mixture was quenched with sat. NH₄Cl, extracted with EtOAc and concentrated in vacuo. The crude material was diluted with acetonitrile and purified by prep-LCMS (Xbridge C18 column, eluting with a gradient of acetonitrile/water containing 0.1% TFA, at flow rate of 60 mL/min). LCMS calculated for C₂₆H₂₉D₃N₇O₄(M+H)⁺: m/z=509.3; found 509.3. ¹H NMR (400 MHz, DMSO) δ 11.97 (s, 1H), 8.76 (d, J=4.4 Hz, 1H), 8.52 (s, 1H), 7.83 (s, 1H), 7.72 (d, J=4.5 Hz, 1H), 7.40 (s, 2H), 5.95 (s, 1H), 4.98 (m, 1H), 4.23 (q, J=6.7 Hz, 1H), 3.56 (s, 3H), 2.51 (m, 3H), 2.33 (m, 1H), 2.28-1.87 (m, 4H), 1.81 (s, 6H), 1.66 (m, 1H).

### Example 30. Methyl ((1R,3R)-3-(3-(methyl-d₃)-2-oxo-6-((3-(1,1,1-trifluoropropan-2-yl)imidazo[1,2-b]pyridazin-6-yl)amino)-2,3-dihydro-1H-imidazo[4,5-c]pyridin-1-yl)cyclopentyl)carbamate

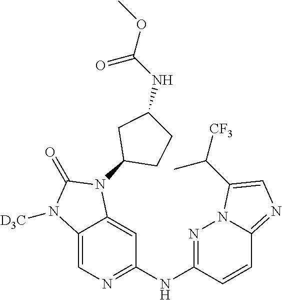 [SMILES: [2H]C([2H])([2H])n1c(=O)n([C@@H]2CC[C@@H](NC(=O)OC)C2)c2cc(Nc3ccc4ncc(C(C)C(F)(F)F)n4n3)ncc21]

### Step 1. Methyl ((1R,3R)-3-(6-((3-iodoimidazo[1,2-b]pyridazin-6-yl)amino)-3-(methyl-d3)-2-oxo-2,3-dihydro-1H-imidazo[4,5-c]pyridin-1-yl)cyclopentyl)carbamate

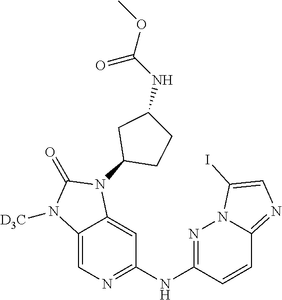 [SMILES: [2H]C([2H])([2H])n1c(=O)n([C@@H]2CC[C@@H](NC(=O)OC)C2)c2cc(Nc3ccc4ncc(I)n4n3)ncc21]

The title compound was prepared according to the procedures described for Example 46, using Intermediate 1 and imidazo[1,2-b]pyridazin-6-amine as starting material. LCMS calculated for C₂₀H₁₉D₃IN₈O₃(M+H)⁺: m/z=552.1; found 552.2.

### Step 2. Methyl ((1R,3R)-3-(3-(methyl-d)-2-oxo-6-((3-(1,1,1-trifluoropropan-2-yl)imidazo[1,2-b]pyridazin-6-yl)amino)-2,3-dihydro-1H-imidazo[4,5-c]pyridin-1-yl)cyclopentyl)carbamate

A vial containing 4,4,5,5-tetramethyl-2-(3,3,3-trifluoroprop-1-en-2-yl)-1,3,2-dioxaborolane (8 mg, 36 μmol), methyl ((1R,3R)-3-(6-((3-iodoimidazo[1,2-b]pyridazin-6-yl)amino)-3-(methyl-d₃)-2-oxo-2,3-dihydro-1H-imidazo[4,5-c]pyridin-1-yl)cyclopentyl)carbamate (10 mg, 18 μmol), (1,1′-bis(diphenylphosphino)ferrocene)-dichloropalladium(II) dichloromethane adduct (3 mg, 3.63 μmol) and potassium carbonate (5 mg, 36 μmol) was evacuated and backfilled with nitrogen three times, followed by the addition of 1,4-dioxane (0.5 mL) and water (50 μL). The vial was sealed and heated to 90° C. for 2 h. After cooling to r.t, the reaction mixture was filtered through a SiliaPrep SPE thiol cartridge (SPE-R51030B-06P) and washed with DCM. The mixture was washed with brine, dried over MgSO₄ and concentrated in vacuo. The crude product was dissolved in MeOH (3.0 mL), followed by the addition of Pd/C (10 wt %, 10 mg). The vial was purged with hydrogen gas for 5 min, and then stirred for 2 h under an atmosphere of hydrogen at 50° C. After cooling to room temperature, the reaction mixture was filtered through Celite and washed with DCM, followed by concentration of the filtrate in vacuo. The mixture was then diluted with acetonitrile and purified with prep-LCMS (Xbridge C18 column, eluting with a gradient of acetonitrile/water containing 0.1% TFA, at flow rate of 60 mL/min). LCMS calculated for C₂₃H₂₃D₃F₃N₈O₃ (M+H)⁺: m/z=522.2; found: 522.2.

### Example 31. Methyl ((1R,3R)-3-(3-(methyl-d₃)-2-oxo-6-((3-(trifluoromethyl)imidazo[1,2-b]pyridazin-6-yl)amino)-2,3-dihydro-1H-imidazo[4,5-c]pyridin-1-yl)cyclopentyl)carbamate

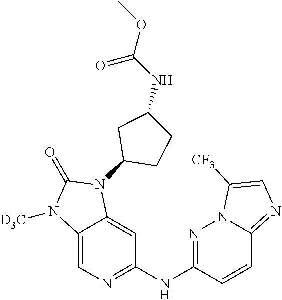 [SMILES: [2H]C([2H])([2H])n1c(=O)n([C@@H]2CC[C@@H](NC(=O)OC)C2)c2cc(Nc3ccc4ncc(C(F)(F)F)n4n3)ncc21]

### Step 1. 6-Chloro-3-(trifluoromethyl)imidazo[1,2-b]pyridazine

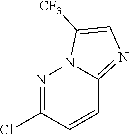 [SMILES: FC(F)(F)c1cnc2ccc(Cl)nn12]

To a microwave vial containing copper(I) iodide (68 mg, 0.358 mmol), potassium fluoride (156 mg, 2.68 mmol), 1,10-phenanthroline (65 mg, 0.358 mmol) and 6-chloro-3-iodoimidazo[1,2-b]pyridazine (250 mg, 0.895 mmol) was added DMSO (1.8 mL). The reaction system was purged with nitrogen. Then trimethyl borate (300 μl, 2.68 mmol) and (trifluoromethyl)trimethylsilane (429 μl, 2.68 mmol) were added via syringe under an atmosphere of nitrogen (gas generation observed). The reaction system was sealed with microwave cap and heated at 80° C. for 4 h on microwave. After cooling to r.t, the reaction mixture was diluted with diethyl ether (5 mL), washed with ammonia aqueous solution and extracted with DCM. The organic phase was washed with brine, dried over MgSO₄ and concentrated in vacuo. The obtained crude product was purified by Biotage Isolera to give the desired product as off-white solid. LCMS calculated for C₇H₄ClF₃N₃ (M+H)⁺: m/z=222.0; found 222.1.

### Step 2. Methyl ((1R,3R)-3-(3-(methyl-d₃)-2-oxo-6-((3-(trifluoromethyl)imidazo[1,2-b]pyridazin-6-yl)amino)-2,3-dihydro-1H-imidazo[4,5-c]pyridin-1-yl)cyclopentyl)carbamate

The title compound was prepared according to the procedures described for Example 46, using Intermediate 1 and 6-chloro-3-(trifluoromethyl)imidazo[1,2-b]pyridazine as starting material. LCMS calculated for C₂₁H₁₉D₃F₃N₈O₃ (M+H)⁺: m/z=494.2; found 494.2.

### Example 32. Methyl (4-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)bicyclo[2.1.1]hexan-1-yl)carbamate

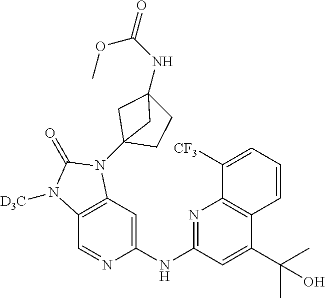 [SMILES: [2H]C([2H])([2H])n1c(=O)n(C23CCC(NC(=O)OC)(C2)C3)c2cc(Nc3cc(C(C)(C)O)c4cccc(C(F)(F)F)c4n3)ncc21]

### Step 1. Tert-Butyl (4-(6-chloro-3-(methyl-d₃)-2-oxo-2,3-dihydro-1H-imidazo[4,5-c]pyridin-1-yl)bicyclo[2.1.1]hexan-1-yl)carbamate

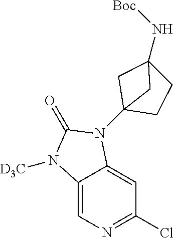 [SMILES: [2H]C([2H])([2H])n1c(=O)n(C23CCC(NC(=O)OC(C)(C)C)(C2)C3)c2cc(Cl)ncc21]

The title compound was prepared in accordance to the procedures described in Intermediate 1, Steps 1-4, with tert-butyl (4-aminobicyclo[2.1.1]hexan-1-yl)carbamate replacing tert-butyl ((1R,3R)-3-aminocyclopentyl)carbamate. LCMS calculated for C₁₈H₂₁D₃ClN₄O₃(M+H)⁺: m/z=382.1; found 382.2

### Step 2. 1-(4-Aminobicyclo[2.1.1]hexan-1-yl)-6-((4-(2-hydroxypropan-2-yl)-8-(trifluoromethyl)quinolin-2-yl)amino)-3-(methyl-d₃)-1,3-dihydro-2H-imidazo[4,5-c]pyridin-2-one

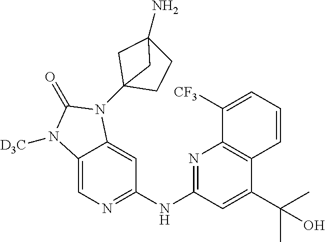 [SMILES: [2H]C([2H])([2H])n1c(=O)n(C23CCC(N)(C2)C3)c2cc(Nc3cc(C(C)(C)O)c4cccc(C(F)(F)F)c4n3)ncc21]

To a flask containing tert-butyl (4-(6-chloro-3-(methyl-d₃)-2-oxo-2,3-dihydro-1H-imidazo[4,5-c]pyridin-1-yl)bicyclo[2.1.1]hexan-1-yl)carbamate (150 mg, 0.393 mmol) 2-(2-amino-8-(trifluoromethyl)quinolin-4-yl)propan-2-ol (159 mg, 0.589 mmol), tris(dibenzylideneacetone)dipalladium(0) (54 mg, 0.059 mmol) and 9,9-dimethyl-4,5-bis(diphenylphosphino)xanthene (68 mg, 0.118 mmol) was added 1,4-dioxane (3.5 ml). The flask was then sparged with N₂ for 3 min. The mixture was stirred at 80° C. for 16 hours, which was then cooled to r.t and diluted with DCM. The reaction mixture was then passed through a celite filter. The filtrate was then diluted with water. The organic phases were then extracted, combined, washed with brine, dried over magnesium sulfate, filtered and concentrated in vacuo. The resulting crude residue was then diluted with DCM (1 mL) and TFA (1 mL), and stirred at room temperature for 15 minutes. The reaction mixture was then diluted with MeOH and purified by prep-LCMS (Xbridge C18 column, eluting with a gradient of acetonitrile/water containing 0.1% TFA, at flow rate of 60 mL/min). LCMS calculated for C₂₆H₂₅D₃F₃N₆O₂ (M+H)⁺: m/z=516.2; found 516.2.

### Step 3. Methyl (4-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)bicyclo[2.1.1]hexan-1-yl)carbamate

To a vial containing 1-(4-aminobicyclo[2.1.1]hexan-1-yl)-6-((4-(2-hydroxypropan-2-yl)-8-(trifluoromethyl)quinolin-2-yl)amino)-3-(methyl-d₃)-1,3-dihydro-2H-imidazo[4,5-c]pyridin-2-one (10 mg, 0.019 mmol), DCM and triethylamine (0.027 ml, 0.194 mmol) was added methyl carbonochloridate (6 mg, 0.058 mmol). The reaction mixture was stirred at room temperature for 15 min. The mixture was then diluted with MeOH and purified by prep-LCMS (Xbridge C18 column, eluting with a gradient of acetonitrile/water containing 0.1% TFA, at flow rate of 60 mL/min). LCMS calculated for C₂₈H₂₇D₃F₃N₆O₄ (M+H)⁺: m/z=574.2; found 574.2.

### Example 33. Methyl (3-(3-(methyl-d₃)-2-oxo-6-((8-(trifluoromethyl)quinolin-2-yl)amino)-2,3-dihydro-1H-imidazo[4,5-c]pyridin-1-yl)bicyclo[1.1.1]pentan-1-yl)carbamate

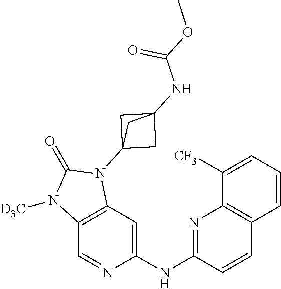 [SMILES: [2H]C([2H])([2H])n1c(=O)n(C23CC(NC(=O)OC)(C2)C3)c2cc(Nc3ccc4cccc(C(F)(F)F)c4n3)ncc21]

### Step 1. Tert-Butyl (3-(6-chloro-3-(methyl-d₃)-2-oxo-2,3-dihydro-1H-imidazo[4,5-c]pyridin-1-yl)bicyclo[1.1.1]pentan-1-yl)carbamate

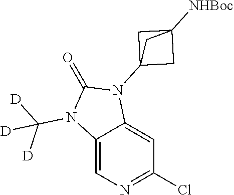 [SMILES: [2H]C([2H])([2H])n1c(=O)n(C23CC(NC(=O)OC(C)(C)C)(C2)C3)c2cc(Cl)ncc21]

The title compound was prepared in accordance to the procedures described for Intermediate 1, steps 1-4 with tert-butyl (3-aminobicyclo[1.1.1]pentan-1-yl)carbamate replacing tert-butyl ((1R,3R)-3-aminocyclopentyl)carbamate. LCMS calculated for C₁₇H₁₉D₃ClN₄O₃(M+H)⁺: m/z=368.2; found 368.2.

### Step 2. 1-(3-Aminobicyclo[1.1.1]pentan-1-yl)-3-(methyl-d₃)-6-((8-(trifluoromethyl)quinolin-2-yl)amino)-1,3-dihydro-2H-imidazo[4,5-c]pyridin-2-one

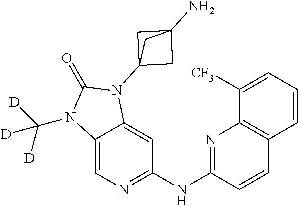 [SMILES: [2H]C([2H])([2H])n1c(=O)n(C23CC(N)(C2)C3)c2cc(Nc3ccc4cccc(C(F)(F)F)c4n3)ncc21]

To a vial containing tert-butyl (3-(6-chloro-3-(methyl-d₃)-2-oxo-2,3-dihydro-1H-imidazo[4,5-c]pyridin-1-yl)bicyclo[1.1.1]pentan-1-yl)carbamate (150 mg, 0.408 mmol) 8-(trifluoromethyl)quinolin-2-amine (130 mg, 0.612 mmol), tris(dibenzylideneacetone)dipalladium(0) (56 mg, 0.061 mmol) and 9,9-dimethyl-4,5-bis(diphenylphosphino)xanthene (71 mg, 0.122 mmol) was added 1,4-dioxane (3.7 mL). The vessel was then sparged with N₂ for 3 minutes and then heated at 80° C. for 16 hours. The reaction was cooled down to r.t, diluted with DCM and filtered over celite, washed with DCM. The combined organic phases were then washed with brine and dried over magnesium sulfate before being purified via Teledyne Isco. The combined organic phases were then concentrated and redissolved in DCM (1 mL) and TFA (1 mL). The mixture was stirred at r.t for 10 minutes. Solvents were removed in vacuo and the crude residue was used in the next step without further purification. LCMS calculated for C₂₂H₁₇D₃F₃N₆O (M+H)⁺: m/z=444.1; found 444.2

### Step 3. Methyl (3-(3-(methyl-d₃)-2-oxo-6-((8-(trifluoromethyl)quinolin-2-yl)amino)-2,3-dihydro-1H-imidazo[4,5-c]pyridin-1-yl)bicyclo[1.1.1]pentan-1-yl)carbamate

To a vial containing 1-(3-aminobicyclo[1.1.1]pentan-1-yl)-3-(methyl-d₃)-6-((8-(trifluoromethyl)quinolin-2-yl)amino)-1,3-dihydro-2H-imidazo[4,5-c]pyridin-2-one (10 mg, 0.023 mmol), DCM and triethylamine (0.031 ml, 0.226 mmol) was added methyl carbonochloridate (6 mg, 0.068 mmol). The reaction was stirred at room temperature for 15 minutes. The mixture was then diluted with MeOH and purified via prep-LCMS (Xbridge C18 column, eluting with a gradient of acetonitrile/water containing 0.1% TFA, at flow rate of 60 mL/min). LCMS calculated for C₂₄H₂₉D₃F₃N₆O₃ (M+H)⁺: 502.2; found 502.2.

### Example 34. Methyl ((1R,3R)-3-(6-((8-(2-cyanopropan-2-yl)-4-(2-hydroxypropan-2-yl)quinolin-2-yl)amino)-3-(methyl-d₃)-2-oxo-2,3-dihydro-1H-imidazo[4,5-c]pyridin-1-yl)cyclopentyl)carbamate

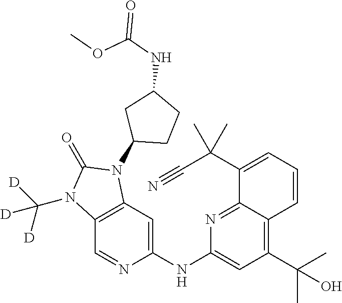 [SMILES: [2H]C([2H])([2H])n1c(=O)n([C@@H]2CC[C@@H](NC(=O)OC)C2)c2cc(Nc3cc(C(C)(C)O)c4cccc(C(C)(C)C#N)c4n3)ncc21]

### Step 1. 2-(8-Bromo-2-((2,4-dimethoxybenzyl)amino)quinolin-4-yl)propan-2-ol

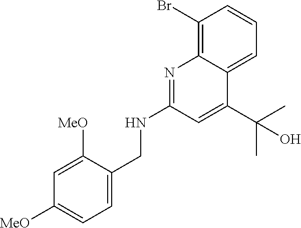 [SMILES: COc1ccc(CNc2cc(C(C)(C)O)c3cccc(Br)c3n2)c(OC)c1]

This compound was prepared according to the procedure described in Intermediate 2 (Steps 1-5), with 7-bromoindoline-2,3-dione replacing 7-(trifluoromethyl)indoline-2,3-dione in Step 1 and with methanol replacing ethanol in Step 2. LCMS calculated for C₂₁H₂₄BrN₂O₃(M+H)⁺: m/z=431.1; found 431.1.

### Step 2. 8-Bromo-N-(2,4-dimethoxybenzyl)-4-(2-(methoxymethoxy)propan-2-yl)quinolin-2-amine

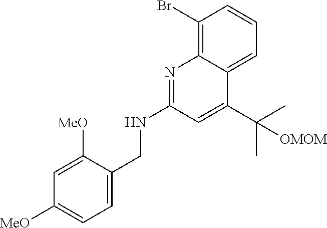

To a vial containing 2-(8-bromo-2-((2,4-dimethoxybenzyl)amino)quinolin-4-yl)propan-2-ol (818 mg, 1.54 mmol) in DCM (15 mL) was added DIPEA (0.8 mL, 4.62 mmol) and then subsequent dropwise addition of chloromethyl methyl ether (0.35 mL, 4.62 mmol), which was then stirred at 23° C. for 1 h. The reaction mixture was quenched with aqueous saturated ammonium chloride solution and then extracted into DCM. The organic phase was washed with brine, dried over MgSO₄ and concentrated in vacuo. The obtained crude product was purified by Biotage Isolera to give the desired product as an orange solid. LCMS calculated for C₂₃H₂₈BrN₂O₄(M+H)⁺: m/z=475.1; found 475.1.

### Step 3. 2-(2-((2,4-Dimethoxybenzyl)amino)-4-(2-(methoxymethoxy)propan-2-yl)quinolin-8-yl)acetonitrile

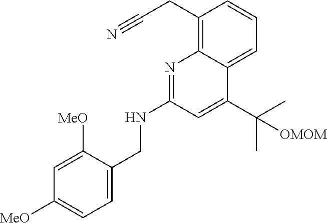

A reaction vial containing 8-bromo-N-(2,4-dimethoxybenzyl)-4-(2-(methoxymethoxy)propan-2-yl)quinolin-2-amine (1.95 g, 4.1 mmol), [1,1′-bis(diphenylphosphino)ferrocene]-dichloropalladium(II) (335 mg, 0.41 mmol), potassium fluoride (715 mg, 12.31 mmol), and 4-(4,4,5,5-tetramethyl-1,3,2-dioxaborolan-2-yl)isoxazole (1.2 g, 6.15 mmol) was evacuated and backfilled with nitrogen. DMSO (21.0 mL) was added to the reaction mixture, which was then stirred at 130° C. for 18 h. The reaction mixture was cooled to r.t and filtered over Celite. A saturated solution of NaCl was added to the solution and extracted into EtOAc. The organic phase was dried over Na₂SO₄ and the solvents were evaporated in vacuo. The obtained crude product was purified by Biotage Isolera to give the desired product as a brown solid. LCMS calculated for C₂₅H₃₀N₃O₄ (M+H)⁺: m/z=436.2; found 436.2.

### Step 4. Tert-Butyl (8-(cyanomethyl)-4-(2-(methoxymethoxy)propan-2-yl)quinolin-2-yl)(2,4-dimethoxybenzyl)carbamate

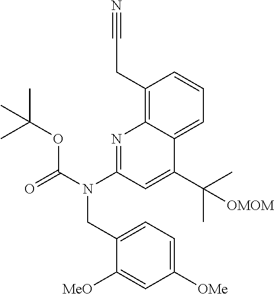

To a reaction vial containing 2-(2-((2,4-dimethoxybenzyl)amino)-4-(2-(methoxymethoxy)propan-2-yl)quinolin-8-yl)acetonitrile (290 mg, 0.67 mmol), DMAP (24 mg, 0.2 mmol) were added DCM (6.7 mL) and di-tert-butyl dicarbonate (0.23 mL, 1.0 mmol). The reaction mixture was allowed to stir at r.t. for 1 hour, after which it was quenched with saturated ammonium chloride and extracted with EtOAc. The organic layer was combined, dried over Na₂SO₄, filtered, and the solvents were evaporated in vacuo. The obtained crude product was purified by Biotage Isolera to give the desired product as a yellow solid. LCMS calculated for C₃₀H₃₈N₃O₆ (M+H)⁺: m/z=536.3; found 536.3.

### Step 5. Tert-Butyl (8-(2-cyanopropan-2-yl)-4-(2-(methoxymethoxy)propan-2-yl)quinolin-2-yl)(2,4-dimethoxybenzyl)carbamate

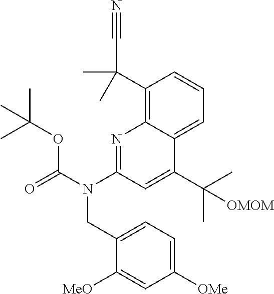

To a reaction vial containing tert-butyl (8-(cyanomethyl)-4-(2-(methoxymethoxy)propan-2-yl)quinolin-2-yl)(2,4-dimethoxybenzyl)carbamate (67 mg, 0.13 mmol) and sodium hydride (20 mg, 0.5 mmol, 60% dispersion in mineral oil) in THF (1.2 mL) was added iodomethane (31 μL, 0.5 mmol) dropwise. The reaction mixture was stirred at 50° C. for 18 h. The reaction mixture was quenched with water and extracted with EtOAc and the organic layer was subsequently washed with water and brine, dried over Na₂SO₄, filtered, and the solvents were evaporated in vacuo. The crude material was purified by Biotage Isolera to give the product. LCMS calculated for C₃₂H₄₂N₃O₆ (M+H)⁺: m/z=564.3; found 564.3.

### Step 6. 2-(2-Amino-4-(2-hydroxypropan-2-yl)quinolin-8-yl)-2-methylpropanenitrile

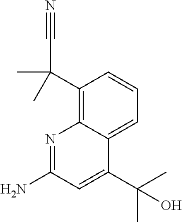 [SMILES: CC(C)(O)c1cc(N)nc2c(C(C)(C)C#N)cccc12]

To a vial containing tert-butyl (8-(2-cyanopropan-2-yl)-4-(2-(methoxymethoxy)propan-2-yl)quinolin-2-yl)(2,4-dimethoxybenzyl)carbamate (70 mg, 0.125 mmol) was added TFA (2 mL) and stirred at 100° C. for 10 min. The mixture was cooled to r.t and concentrated in vacuo and azeotroped with acetonitrile. The crude material was used in the next step without further purification. LCMS calculated for C₁₆H₂₀N₃O (M+H)⁺: m/z=270.2; found 270.2.

### Step 7. Methyl ((1R,3R)-3-(6-((8-(2-cyanopropan-2-yl)-4-(2-hydroxypropan-2-yl)quinolin-2-yl)amino)-3-(methyl-d₃)-2-oxo-2,3-dihydro-1H-imidazo[4,5-c]pyridin-1-yl)cyclopentyl)carbamate

To a reaction vial were added 2-(2-amino-4-(2-hydroxypropan-2-yl)quinolin-8-yl)-2-methylpropanenitrile (30 mg, 0.1 mmol), methyl ((1R,3R)-3-(6-chloro-3-(methyl-d₃)-2-oxo-2,3-dihydro-1H-imidazo[4,5-c]pyridin-1-yl)cyclopentyl)carbamate (36 mg, 0.1 mmol, Intermediate 1), xantphos-Pd-G3 (11 mg, 0.01 mmol), and cesium carbonate (109 mg, 0.33 mmol). This was evacuated and backfilled with nitrogen 3 times. Subsequently 1,4-dioxane (1.0 mL) was added. The vial was sealed and heated to 100° C. for 16 h. Upon completion, the reaction was diluted with DCM, filtered through Celite and concentrated in vacuo. The crude material was diluted with acetonitrile and purified by prep-LCMS (Xbridge C18 column, eluting with a gradient of acetonitrile/water containing 0.1% TFA, at flow rate of 60 mL/min. LCMS calculated for C₃₀H₃₃D₃N₇O₄ (M+H)⁺: m/z=561.3; found 561.3.

### Example 35. Methyl ((1R,3R)-3-(3-(methyl-d₃)-6-((8-nitroquinolin-2-yl)amino)-2-oxo-2,3-dihydro-1H-imidazo[4,5-c]pyridin-1-yl)cyclopentyl)carbamate

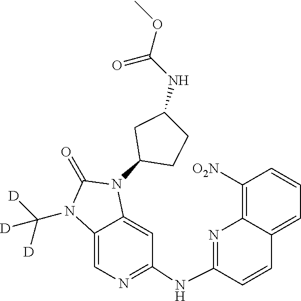 [SMILES: [2H]C([2H])([2H])n1c(=O)n([C@@H]2CC[C@@H](NC(=O)OC)C2)c2cc(Nc3ccc4cccc([N+](=O)[O-])c4n3)ncc21]

### Step 1. 8-Nitroquinolin-2-amine

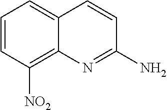 [SMILES: Nc1ccc2cccc([N+](=O)[O-])c2n1]

To a vial containing 2-chloro-8-nitroquinoline (200 mg, 0.96 mmol) and (2,4-dimethoxyphenyl)methanamine (321 mg, 1.9 mmol) in DMSO (2.7 mL) was added triethylamine (0.3 mL, 2.4 mmol), which was then stirred at 110° C. for 1 h. The reaction mixture was cooled to r.t and water was added. The solid was then filtered and washed with water. The crude material was transferred to a vial to which TFA (2 mL) was added. The reaction vial was stirred at 100° C. for 10 min. The mixture was cooled to r.t and concentrated in vacuo and azeotroped with acetonitrile. The obtained crude product was purified by Biotage Isolera to give the desired product as an orange solid. LCMS calculated for C₉H₈N₃O₂(M+H)⁺: m/z=190.1; found 190.1.

### Step 2. Methyl ((1R,3R)-3-(3-(methyl-d₃)-6-((8-nitroquinolin-2-yl)amino)-2-oxo-2,3-dihydro-1H-imidazo[4,5-c]pyridin-1-yl)cyclopentyl)carbamate

This compound was prepared according to the procedure described in Example 34 Step 7, with 8-nitroquinolin-2-amine replacing 2-(2-amino-4-(2-hydroxypropan-2-yl)quinolin-8-yl)-2-methylpropanenitrile. LCMS calculated for C₂₃H₂₁D₃N₇O₅(M+H)⁺: m/z=481.2; found 481.2.

### Example 36. Methyl ((1R,3R)-3-(6-((8-cyano-1,5-naphthyridin-2-yl)amino)-3-(methyl-d₃)-2-oxo-2,3-dihydro-1H-imidazo[4,5-c]pyridin-1-yl)cyclopentyl)carbamate

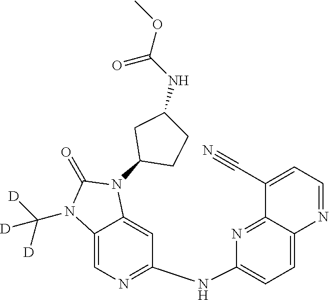 [SMILES: [2H]C([2H])([2H])n1c(=O)n([C@@H]2CC[C@@H](NC(=O)OC)C2)c2cc(Nc3ccc4nccc(C#N)c4n3)ncc21]

### Step 1. 8-Chloro-1,5-naphthyridin-2-amine

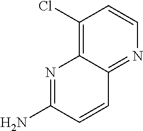 [SMILES: Nc1ccc2nccc(Cl)c2n1]

To a microwave vial containing 2,8-dichloro-1,5-naphthyridine (150 mg, 0.75 mmol) and 1,4-dioxane (1.5 mL) was added ammonium hydroxide (3 mL), which was then microwaved at 120° C. for 2 h. The reaction mixture was cooled to r.t and extracted with EtOAc. The organic layer was dried over Na₂SO₄, filtered, and the solvents were evaporated in vacuo. The crude material was used in the next step without further purification. LCMS calculated for C₈H₇ClN₃ (M+H)⁺: m/z=180.0; found 180.0.

### Step 2. 6-Amino-1,5-naphthyridine-4-carbonitrile

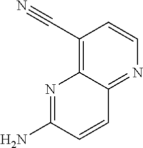 [SMILES: N#Cc1ccnc2ccc(N)nc12]

To a reaction vial was added 8-chloro-1,5-naphthyridin-2-amine (135 mg, 0.75 mmol), zinc cyanide (177 mg, 1.5 mmol), and xantphos-Pd-G3 (71 mg, 0.08 mmol), This was evacuated and backfilled with nitrogen 3 times. Subsequently dimethylacetamide (0.75 mL) was added. The vial was sealed and heated to 85° C. for 16 h. Upon completion, the reaction quenched with a solution of saturated ammonium chloride and extracted with EtOAc. The organic phase was washed with brine and dried over Na₂SO₄. The solvents were evaporated in vacuo and the crude material was used in the next step without further purification. LCMS calculated for C₉H₇N₄ (M+H)⁺: m/z=171.1; found 171.1.

### Step 3. Methyl ((1R,3R)-3-(6-((8-cyano-1,5-naphthyridin-2-yl)amino)-3-(methyl-d₃)-2-oxo-2,3-dihydro-1H-imidazo[4,5-c]pyridin-1-yl)cyclopentyl)carbamate

This compound was prepared according to the procedure described in Example 34 Step 7, with 6-amino-1,5-naphthyridine-4-carbonitrile replacing 2-(2-amino-4-(2-hydroxypropan-2-yl)quinolin-8-yl)-2-methylpropanenitrile. LCMS calculated for C₂₃H₂₀D₃N₈O₃(M+H)⁺: m/z=462.2; found 462.2.

### Example 37. Methyl ((1R,3R)-3-(6-((8-cyanoquinoxalin-2-yl)amino)-3-(methyl-d₃)-2-oxo-2,3-dihydro-1H-imidazo[4,5-c]pyridin-1-yl)cyclopentyl)carbamate

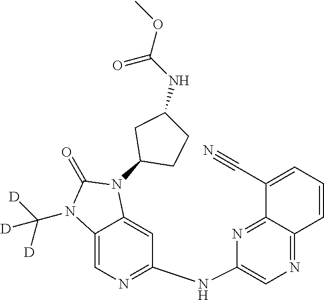 [SMILES: [2H]C([2H])([2H])n1c(=O)n([C@@H]2CC[C@@H](NC(=O)OC)C2)c2cc(Nc3cnc4cccc(C#N)c4n3)ncc21]

This compound was prepared according to the procedure described in Example 36 (Step 1-3), with 8-bromo-2-chloroquinoxaline replacing 2,8-dichloro-1,5-naphthyridine in Step 1. LCMS calculated for C₂₃H₂₀D₃N₈O₃(M+H)⁺: m/z=462.2; found 462.2.

### Example 38. Methyl ((1R,3R)-3-(6-((8-(difluoromethyl)quinolin-2-yl)amino)-3-methyl-2-oxo-2,3-dihydro-1H-imidazo[4,5-c]pyridin-1-yl)cyclopentyl)carbamate

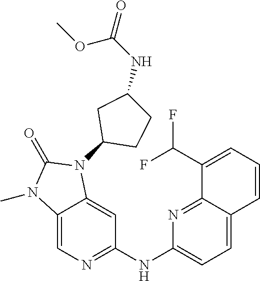 [SMILES: COC(=O)N[C@@H]1CC[C@@H](n2c(=O)n(C)c3cnc(Nc4ccc5cccc(C(F)F)c5n4)cc32)C1]

### Step 1. 2-Chloro-8-(difluoromethyl)quinoline

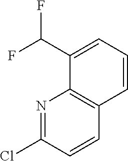 [SMILES: FC(F)c1cccc2ccc(Cl)nc12]

To a reaction vial was added 2-chloroquinoline-8-carbaldehyde (50 mg, 0.75 mmol) dissolved in DCM (2.6 mL) and cooled to −78° C. Deoxofluor (0.8 mL, 0.8 mmol, 1 M in DCM) was added dropwise and the reaction was stirred for 1 h and allowed to warm to room temperature over this time. The reaction was quenched with water and extracted with DCM. The organic phase was dried over Na₂SO₄ and the solvent was evaporated in vacuo. The crude material was used in the next step without further purification. LCMS calculated for C₁₀H₇ClF₂N (M+H)⁺: m/z=214.0; found 214.0.

### Step 2. 8-(Difluoromethyl)quinolin-2-amine

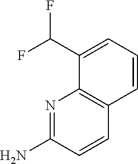 [SMILES: Nc1ccc2cccc(C(F)F)c2n1]

This compound was prepared according to the procedure described in Example 36 Step 1 with 2-chloro-8-(difluoromethyl)quinoline replacing 2,8-dichloro-1,5-naphthyridine. LCMS calculated for C₁₀H₉F₂N₂ (M+H)⁺: m/z=195.1; found 195.1.

### Step 3. Methyl ((1R,3R)-3-(6-((8-(difluoromethyl)quinolin-2-yl)amino)-3-methyl-2-oxo-2,3-dihydro-1H-imidazo[4,5-c]pyridin-1-yl)cyclopentyl)carbamate

To a reaction vial were added 8-(difluoromethyl)quinolin-2-amine (9.0 mg, 0.05 mmol), methyl ((1R,3R)-3-(6-chloro-3-methyl-2-oxo-2,3-dihydro-1H-imidazo[4,5-c]pyridin-1-yl)cyclopentyl)carbamate (10 mg, 0.03 mmol), Xantphos-Pd-G3 (3 mg, 3.0 mmol), and cesium carbonate (30 mg, 0.1 mmol). This was evacuated and backfilled with nitrogen 3 times. Subsequently 1,4-dioxane (0.5 mL) was added. The vial was sealed and heated to 100° C. for 16 h. Upon completion, the reaction was diluted with DCM, filtered through Celite and concentrated in vacuo. The crude material was diluted with acetonitrile and purified by prep-LCMS (Xbridge C18 column, eluting with a gradient of acetonitrile/water containing 0.1% TFA, at flow rate of 60 mL/min. LCMS calculated for C₂₄H₂₅F₂N₆O₃(M+H)⁺: m/z=483.2; found 483.2.

### Example 39. Methyl ((1R,3R)-3-(3-methyl-6-((8-methylquinolin-2-yl)amino)-2-oxo-2,3-dihydro-1H-imidazo[4,5-c]pyridin-1-yl)cyclopentyl)carbamate

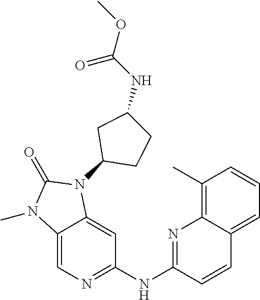 [SMILES: COC(=O)N[C@@H]1CC[C@@H](n2c(=O)n(C)c3cnc(Nc4ccc5cccc(C)c5n4)cc32)C1]

The title compound was prepared according to the procedure described in Example 38 Step 3 with 8-methylquinolin-2-amine replacing 8-(difluoromethyl)quinolin-2-amine. LCMS calculated for C₂₄H₂₆N₆O₃ (M+H)⁺: m/z=447.2; found 447.2

### Example 40. Methyl ((1S,3S)-3-(6-((8-chloroquinolin-2-yl)amino)-3-methyl-2-oxo-2,3-dihydro-1H-imidazo[4,5-c]pyridin-1-yl)cyclopentyl)carbamate

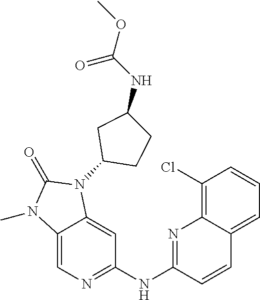 [SMILES: COC(=O)N[C@H]1CC[C@H](n2c(=O)n(C)c3cnc(Nc4ccc5cccc(Cl)c5n4)cc32)C1]

The title compound was prepared according to the procedure described in Example 38 Step 3 with 8-chloroquinolin-2-amine replacing 8-(difluoromethyl)quinolin-2-amine. LCMS calculated for C₂₃H₂₄ClN₆O₃(M+H)⁺: m/z=467.2; found 467.2

### Example 41. Methyl ((1R,3R)-3-(3-methyl-2-oxo-6-((8-(trifluoromethyl)quinolin-2-yl)amino)-2,3-dihydro-1H-imidazo[4,5-c]pyridin-1-yl)cyclopentyl)carbamate

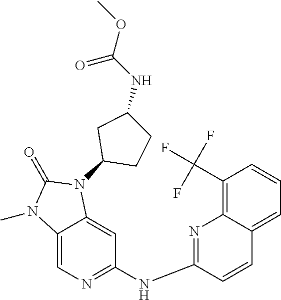 [SMILES: COC(=O)N[C@@H]1CC[C@@H](n2c(=O)n(C)c3cnc(Nc4ccc5cccc(C(F)(F)F)c5n4)cc32)C1]

This compound was prepared according to the procedure described in Example 38 Step 3 with 8-(trifluoromethyl)quinolin-2-amine replacing 8-(difluoromethyl)quinolin-2-amine. LCMS calculated for C₂₄H₂₄F₃N₆O₃(M+H)⁺: m/z=501.2; found 501.2

### Example 42. Methyl ((1R,3R)-3-(6-((3-cyanoimidazo[1,2-b]pyridazin-6-yl)amino)-3-methyl-2-oxo-2,3-dihydro-1H-imidazo[4,5-c]pyridin-1-yl)cyclopentyl)carbamate

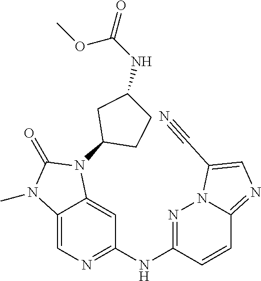 [SMILES: COC(=O)N[C@@H]1CC[C@@H](n2c(=O)n(C)c3cnc(Nc4ccc5ncc(C#N)n5n4)cc32)C1]

### Step 1. 6-Aminoimidazo[1,2-b]pyridazine-3-carbonitrile

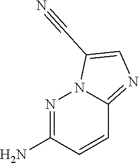 [SMILES: N#Cc1cnc2ccc(N)nn12]

To a reaction vial was added 3-bromoimidazo[1,2-b]pyridazin-6-amine (20 mg, 0.1 mmol), zinc cyanide (22 mg, 0.2 mmol), and Xantphos-Pd-G3 (9 mg, 9.4 μmol), This was evacuated and backfilled with nitrogen 3 times. Subsequently dimethylacetamide (0.5 mL) was added. The vial was sealed and heated to 85° C. for 16 h. Upon completion, the reaction was quenched with a solution of saturated ammonium chloride and extracted with EtOAc. The organic phase was washed with brine and dried over Na₂SO₄. The solvents were evaporated in vacuo and the crude material was used in the next step without further purification. LCMS calculated for C₇H₆N₅ (M+H)⁺: m/z=160.1; found 160.1.

### Step 2. Methyl ((1R,3R)-3-(6-((3-cyanoimidazo[1,2-b]pyridazin-6-yl)amino)-3-methyl-2-oxo-2,3-dihydro-1H-imidazo[4,5-c]pyridin-1-yl)cyclopentyl)carbamate

The title compound was prepared according to the procedure described in Example 38 Step 3, with 6-aminoimidazo[1,2-b]pyridazine-3-carbonitrile replacing 8-(difluoromethyl)quinolin-2-amine. LCMS calculated for C₂₁H₂₂N₉O₃ (M+H)⁺: m/z=448.2; found 448.2.

### Example 43. Methyl ((1R,3R)-3-(6-((8-cyanoquinolin-2-yl)amino)-3-methyl-2-oxo-2,3-dihydro-1H-imidazo[4,5-c]pyridin-1-yl)cyclopentyl)carbamate

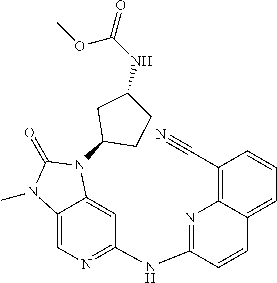 [SMILES: COC(=O)N[C@@H]1CC[C@@H](n2c(=O)n(C)c3cnc(Nc4ccc5cccc(C#N)c5n4)cc32)C1]

The title compound was prepared according to the procedure described in Example 42, with 8-bromoquinolin-2-amine replacing 3-bromoimidazo[1,2-b]pyridazin-6-amine. LCMS calculated for C₂₄H₂₄N₇O₃ (M+H)⁺: m/z=458.2; found 458.2.

### Example 44. Methyl ((1R,3R)-3-(6-((7-cyanoquinolin-2-yl)amino)-3-methyl-2-oxo-2,3-dihydro-1H-imidazo[4,5-c]pyridin-1-yl)cyclopentyl)carbamate

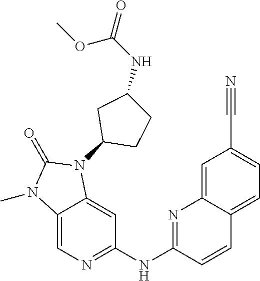 [SMILES: COC(=O)N[C@@H]1CC[C@@H](n2c(=O)n(C)c3cnc(Nc4ccc5ccc(C#N)cc5n4)cc32)C1]

The title compound was prepared according to the procedure described in Example 42, with 7-bromoquinolin-2-amine replacing 3-bromoimidazo[1,2-b]pyridazin-6-amine. LCMS calculated for C₂₄H₂₄N₇O₃ (M+H)⁺: m/z=458.2; found 458.2.

### Example 45. Methyl ((1R,3R)-3-(6-((6-cyanoquinolin-2-yl)amino)-3-methyl-2-oxo-2,3-dihydro-1H-imidazo[4,5-c]pyridin-1-yl)cyclopentyl)carbamate

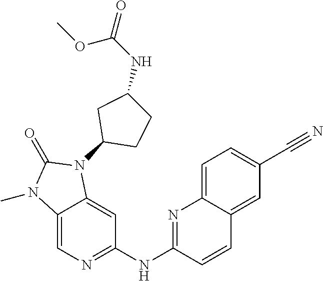 [SMILES: COC(=O)N[C@@H]1CC[C@@H](n2c(=O)n(C)c3cnc(Nc4ccc5cc(C#N)ccc5n4)cc32)C1]

The title compound was prepared according to the procedure described in Example 42, Step 2, with 6-bromoquinolin-2-amine replacing 3-bromoimidazo[1,2-b]pyridazin-6-amine. LCMS calculated for C₂₄H₂₄N₇O₃ (M+H)⁺: m/z=458.2; found 458.2.

### Example 46. Methyl ((1R,3R)-3-(6-((3-iodo-2,8-dimethylimidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate

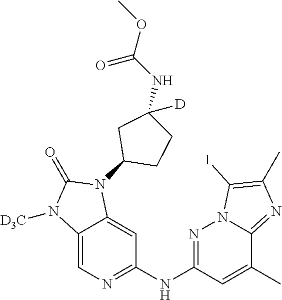 [SMILES: [2H]C([2H])([2H])n1c(=O)n([C@@H]2CC[C@@]([2H])(NC(=O)OC)C2)c2cc(Nc3cc(C)c4nc(C)c(I)n4n3)ncc21]

### Step 1. Tert-Butyl ((1R,3S)-3-hydroxycyclopentyl-3-d)carbamate

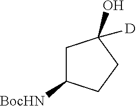 [SMILES: [2H][C@]1(O)CC[C@@H](NC(=O)OC(C)(C)C)C1]

To a flask containing tert-butyl (R)-(3-oxocyclopentyl)carbamate (10.0 g, 50.2 mmol) in MeOH (100 mL) at 0° C. was added sodium borodeuteride (1.26 g, 30.1 mmol) in portions (over 10 times) during 30 min. The reaction mixture was stirred at 0° C. for 3 h. The reaction mixture was concentrated, diluted with DCM (250 mL), and washed with 1.0 M sodium hydroxide solution. After extraction with DCM (100 mL) for 3 times, the organic layer was combined, dried over MgSO₄ and concentrated in vacuo. The crude material was purified by CombiFlash (earlier peak) to give the desired product as a white solid. LCMS calculated for C₆H₁₁DNO₃ (M+H−t-Bu)⁺. m/z=147.1; found 147.1.

### Step 2. Tert-Butyl ((1R,3R)-3-(1,3-dioxoisoindolin-2-yl)cyclopentyl-3-d)carbamate

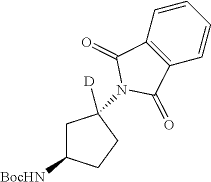 [SMILES: [2H][C@@]1(N2C(=O)c3ccccc3C2=O)CC[C@@H](NC(=O)OC(C)(C)C)C1]

To a flask containing tert-butyl ((1R,3R)-3-hydroxycyclopentyl-3-d)carbamate (3.98 g, 19.7 mmol) in THF (79 ml) was added triphenylphosphine (6.19 g, 23.61 mmol) and isoindoline-1,3-dione (4.34 g, 29.5 mmol). The reaction mixture was cooled down to 0° C. and the diisopropyl (E)-diazene-1,2-dicarboxylate (4.77 g, 4.65 mL, 23.61 mmol) was added dropwise to the flask. The reaction mixture was stirred at 0° C. to rt for 3 h. The reaction mixture was quenched with water (30 mL) and extraction with DCM. The organic layer was combined, dried over MgSO₄ and concentrated in vacuo. The crude material was purified by Biotage Isolera. LCMS calculated for C₁₈H₂₂DN₂O₄(M+H)⁺: m/z=332.2; found 332.1.

### Step 3. Tert-Butyl ((1R,3R)-3-aminocyclopentyl-3-d)carbamate

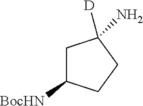 [SMILES: [2H][C@@]1(N)CC[C@@H](NC(=O)OC(C)(C)C)C1]

To a flask containing tert-butyl ((1R,3R)-3-(1,3-dioxoisoindolin-2-yl)cyclopentyl-3-d)carbamate (5.5 g, 16.60 mmol) in ethanol (55.3 mL) was added hydrazine (1.06 g, 1.0 mL, 33.2 mmol). The reaction mixture was stirred at 80° C. for 1.5 h with white solid generation during the reaction. After cooling down to r.t, the reaction mixture was filtered and washed with ethanol (100 mL) and DCM (100 mL) to filter out the white solid as side product. The filtrate was collected and concentrated in vacuo to give crude product. The obtained crude product was used in the next step without further purification. LCMS calculated for C₆H₁₂DN₂O₂(M+H−t-Bu)⁺: m/z=146.1; found: 146.1.

### Step 4. Methyl ((1R,3R)-3-aminocyclopentyl-1-d)carbamate

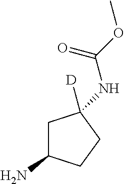

To a flask containing tert-butyl ((1R,3R)-3-aminocyclopentyl-3-d)carbamate (5.0 g, 24.8 mmol) and DIPEA (3.53 g, 4.77 mL, 27.3 mmol) in DCM (200 mL) and MeOH (5 mL) at 0° C. was added methyl chloroformate (1.92 mL, 24.8 mmol) dropwise. After stirring at 0° C. for 10 min., the reaction was quenched with MeOH (10 mL) and concentrated in vacuo. The residue was re-dissolved in DCM (200 mL) and washed with sodium bicarbonate saturated solution, dried over MgSO₄ and concentrated in vacuo. The crude material was treated with 4N HCl in 1,4-dioxane (20 mL). After stirring at r.t for 1 h, the reaction mixture was concentrated in vacuo. The obtained crude product was used in the next step without further purification. LCMS calculated for C₇H₁₄DN₂O₂(M+H)⁺: m/z=160.1; found: 160.1.

### Step 5. Methyl ((1R,3R)-3-(6-chloro-3-(methyl-d₃)-2-oxo-2,3-dihydro-1H-imidazo[4,5-c]pyridin-1-yl)cyclopentyl-1-d)carbamate

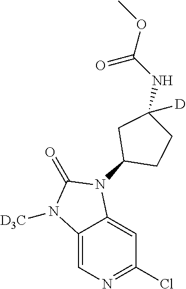 [SMILES: [2H]C([2H])([2H])n1c(=O)n([C@@H]2CC[C@@]([2H])(NC(=O)OC)C2)c2cc(Cl)ncc21]

The title compound was prepared according to the procedures described for Intermediate 1, using methyl ((1R,3R)-3-aminocyclopentyl-1-d)carbamate as starting material. LCMS calculated for C₁₄H₁₄D₄ClN₄O₃(M+H)⁺: m/z=329.1; found: 329.1.

### Step 6. 2,8-Dimethylimidazo[1,2-b]pyridazin-6-amine

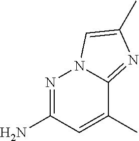

A vial containing tert-butyl carbamate (387 mg, 3.30 mmol), 6-chloro-2,8-dimethylimidazo[1,2-b]pyridazine (200 mg, 1.10 mmol), cesium carbonate (718 mg, 2.202 mmol), 4,5-bis(diphenylphosphino)-9,9-dimethylxanthene (102 mg, 0.176 mmol) and tris(dibenzylideneacetone)dipalladium(0) (81 mg, 0.088 mmol) was evacuated and backfilled with nitrogen three times, followed by the addition of 1,4-dioxane (5.5 mL). The vial was sealed and heated to 100° C. for 6 h. After cooling to r.t, the mixture was filtered through a SiliaPrep SPE thiol cartridge (SPE-R51030B-06P) and washed with DCM. The mixture was washed with brine, dried over MgSO₄ and concentrated in vacuo. The crude material was redissolved in DCM (3.0 mL) and TFA (3.0 mL). After stirring at 40° C. for 0.5 h, the reaction mixture was concentrated in vacuo. The crude material was redissolved in DCM (20 mL) and the pH of the mixture was adjusted to ˜10 with ammonia aqueous solution and then extracted into DCM. The organic phase was washed with brine, dried over MgSO₄ and concentrated in vacuo. The obtained crude product was purified by Biotage Isolera to give the desired product as off-white solid. LCMS calculated for C₈H₁N₄ (M+H)⁺: m/z=163.1; found: 163.0.

### Step 7. Methyl ((1R,3R)-3-(6-((2,8-dimethylimidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate

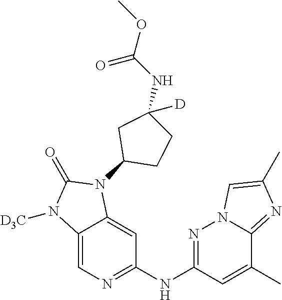 [SMILES: [2H]C([2H])([2H])n1c(=O)n([C@@H]2CC[C@@]([2H])(NC(=O)OC)C2)c2cc(Nc3cc(C)c4nc(C)cn4n3)ncc21]

A vial containing methyl ((1R,3R)-3-(6-chloro-3-(methyl-d₃)-2-oxo-2,3-dihydro-1H-imidazo[4,5-c]pyridin-1-yl)cyclopentyl-1-d)carbamate (100 mg, 0.304 mmol), 2,8-dimethylimidazo[1,2-b]pyridazin-6-amine (49 mg, 0.304 mmol), cesium carbonate (198 mg, 0.608 mmol), 4,5-bis(diphenylphosphino)-9,9-dimethylxanthene (46 mg, 0.079 mmol) and tris(dibenzylideneacetone)dipalladium(0) (36 mg, 0.040 mmol) was evacuated and backfilled with nitrogen three times, followed by the addition of 1,4-dioxane (3.0 mL). The vial was sealed and heated to 100° C. for 6 h. After cooling to r.t, the mixture was filtered through a SiliaPrep SPE thiol cartridge (SPE-R51030B-06P) and washed with DCM. The mixture was washed with brine, dried over MgSO₄ and concentrated in vacuo. The obtained crude product was purified by Biotage Isolera to give the desired product as off-white solid. LCMS calculated for C₂₂H₂₃D₄N₈O₃(M+H)⁺: m/z=455.2; found: 455.2

### Step 8. Methyl ((1R,3R)-3-(6-((3-iodo-2,8-dimethylimidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate

To a vial containing methyl ((1R,3R)-3-(6-((2,8-dimethylimidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate (140 mg, 0.308 mmol) in acetonitrile (1.5 mL) was added N-iodosuccinimide (87 mg, 0.385 mmol) and 3 drops of DMF. The reaction was stirred at 80° C. for 20 min. After cooling to r.t, the mixture was concentrated in vacuo. The mixture was then diluted with acetonitrile and purified with prep-LCMS (Xbridge C18 column, eluting with a gradient of acetonitrile/water containing 0.1% TFA, at flow rate of 60 mL/min). LCMS calculated for C₂₂H₂₂D₄IN₈O₃(M+H)⁺: m/z=581.1; found: 581.2.

### Example 47. Methyl ((1R,3R)-3-(6-((5-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

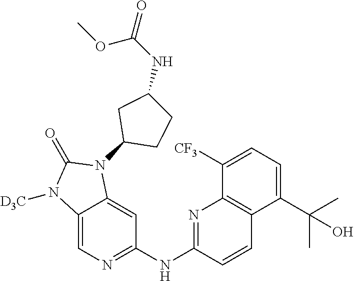 [SMILES: [2H]C([2H])([2H])n1c(=O)n([C@@H]2CC[C@@H](NC(=O)OC)C2)c2cc(Nc3ccc4c(C(C)(C)O)ccc(C(F)(F)F)c4n3)ncc21]

### Step 1. N-(5-Chloro-2-(trifluoromethyl)phenyl)-3,3-dimethoxypropanamide

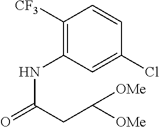 [SMILES: COC(CC(=O)Nc1cc(Cl)ccc1C(F)(F)F)OC]

To a solution of 5-chloro-2-(trifluoromethyl)aniline (1.00 g, 5.11 mmol) and methyl 3,3-dimethoxypropanoate (0.87 mL, 6.14 mmol) in THF (10 mL) at 0° C. was slowly added NaHMDS (7.7 mL, 7.7 mmol). The reaction contents were then allowed to warm to 23° C. and stirred for 3 h. Upon completion, the reaction mixture was quenched with sat. NH₄Cl (20 mL) and extracted three times with EtOAc (20 mL). The organic layers were combined and dried over Na₂SO₄ before being concentrated in vacuo to yield the desired crude product. LCMS calculated for C₁₂H₁₄ClF₃NO₃ (M+H)⁺: m/z=312.1; found 312.1.

### Step 2. 5-Chloro-8-(trifluoromethyl)quinolin-2(1H)-one

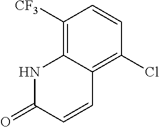 [SMILES: O=c1ccc2c(Cl)ccc(C(F)(F)F)c2[nH]1]

To a solution of N-(5-chloro-2-(trifluoromethyl)phenyl)-3,3-dimethoxypropanamide (1.60 g, 5.13 mmol) in DCM (3.4 mL) was added dropwise to H₂SO₄ (14 mL). The reaction mixture was then heated to 80° C. for 20 min. Upon completion, the reaction contents were cooled to r.t and the DCM was removed in vacuo. The crude residue was redissolved in EtOAc (15 mL) and was washed three times with sat. NaHCO₃ (15 mL). The organic layer was then dried over Na₂SO₄ and was concentrated in vacuo. The crude material was purified by Biotage Isolera. LCMS calculated for C₁₀H₆ClF₃NO (M+H)⁺: m/z=248.0; found 248.0.

### Step 3. 2,5-Dichloro-8-(trifluoromethyl)quinoline

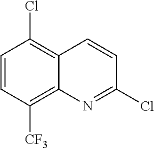 [SMILES: FC(F)(F)c1ccc(Cl)c2ccc(Cl)nc12]

To 5-chloro-8-(trifluoromethyl)quinolin-2(1H)-one (883 mg, 3.57 mmol) was added phosphoryl trichloride (8.3 mL, 89 mmol). The reaction was then heated to 100° C. and was allowed to stir for 30 min. Upon completion, the contents were cooled, then slowly added to ice water. The quenched reaction mixture was then allowed to stir for 1 h to produce a fine precipitate. The precipitate was then filtered and dried over vacuum for 6 h to deliver the desired product without further purification. LCMS calculated for C₁₀H₅Cl₂F₃N (M+H)⁺: m/z=266.0; found 266.0.

### Step 4. 5-Chloro-N-(2,4-dimethoxybenzyl)-8-(trifluoromethyl)quinolin-2-amine

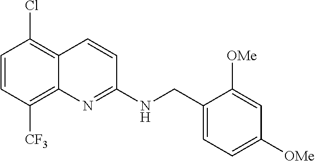 [SMILES: COc1ccc(CNc2ccc3c(Cl)ccc(C(F)(F)F)c3n2)c(OC)c1]

To a solution of 2,5-dichloro-8-(trifluoromethyl)quinoline (510 mg, 1.92 mmol) in DMSO (5.5 mL) was added (2,4-dimethoxyphenyl)methanamine (0.58 mL, 3.83 mmol) and triethylamine (0.67 mL, 4.79 mmol). The reaction was then heated to 110° C. for 1 h. Upon completion, the reaction contents were cooled to 23° C. before diluting with EtOAc (30 mL) and washing the organic layer three times with H₂O (15 mL). The organic layer was dried over Na₂SO₄, concentrated in vacuo. The crude material was purified by Biotage Isolera. LCMS calculated for C₁₉H₁₇ClF₃N₂O₂ (M+H)⁺: m/z=397.1; found 397.1.

### Step 5. Tert-Butyl (5-chloro-8-(trifluoromethyl)quinolin-2-yl)(2,4-dimethoxybenzyl)carbamate

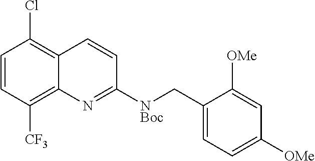 [SMILES: COc1ccc(CN(C(=O)OC(C)(C)C)c2ccc3c(Cl)ccc(C(F)(F)F)c3n2)c(OC)c1]

To a solution of 5-chloro-N-(2,4-dimethoxybenzyl)-8-(trifluoromethyl)quinolin-2-amine (336 mg, 0.848 mmol) in DCM (8.5 mL) was added di-tert-butyl dicarbonate (278 mg, 1.272 mmol), N,N-dimethylpyridin-4-amine (31 mg, 0.254 mmol), and Et₃N (0.47 mL, 3.39 mmol). The resulting reaction mixture was allowed to stir for 4 h. Upon completion, the reaction contents were concentrated directly before purification by flash column chromatography to provide the desired product. LCMS calculated for C₂₄H₂₅ClF₃N₂O₄ (M+H)⁺: m/z=497.1; found 497.2.

### Step 6. Tert-Butyl (5-cyano-8-(trifluoromethyl)quinolin-2-yl)(2,4-dimethoxybenzyl)carbamate

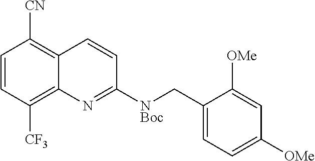 [SMILES: COc1ccc(CN(C(=O)OC(C)(C)C)c2ccc3c(C#N)ccc(C(F)(F)F)c3n2)c(OC)c1]

To tert-butyl (5-chloro-8-(trifluoromethyl)quinolin-2-yl)(2,4-dimethoxybenzyl)carbamate (368 mg, 0.740 mmol) was added zinc cyanide (174 mg, 1.480 mmol), Sphos (152 mg, 0.370 mmol), and Pd₂(dba)₃ (169 mg, 0.185 mmol) before dissolving in DMF (2.9 mL) and water (29 μL). The reaction vessel was then sealed and subjected to microwave irradiation at 170° C. for 35 min. Upon completion, the reaction was diluted with EtOAc (15 mL) and washing the organic layer three times with H₂O (10 mL). The organic layer was dried over Na₂SO₄, concentrated in vacuo, and purified by flash column chromatography to provide the desired product. LCMS calculated for C₂₅H₂₅F₃N₃O₄(M+H)⁺: m/z=488.2; found 488.1.

### Step 7. Methyl 2-amino-8-(trifluoromethyl)quinoline-5-carboxylate

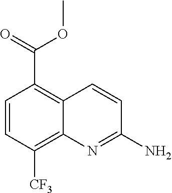 [SMILES: COC(=O)c1ccc(C(F)(F)F)c2nc(N)ccc12]

To a solution of tert-butyl (5-cyano-8-(trifluoromethyl)quinolin-2-yl)(2,4-dimethoxybenzyl)carbamate (350 mg, 0.718 mmol) in EtOH (3.3 mL) and water (0.33 mL) was added NaOH (144 mg, 3.59 mmol). The reaction was sealed and heated to 100° C. for 14 h. Upon completion, the reaction mixture was quenched by the addition of 1M HCl (3.6 mL) and was subsequently extracted three times with EtOAc (10 mL). The combined organic layers were dried over Na₂SO₄ and concentrated in vacuo to yield the crude product which was carried forward without further purification. The resulting crude 2-((tert-butoxycarbonyl)(2,4-dimethoxybenzyl)amino)-8-(trifluoromethyl)quinoline-5-carboxylic acid (354 mg, 0.700 mmol) was dissolved in MeOH (3.1 mL) and H₂SO₄ (0.39 mL). The reaction mixture was then heated to 100° C. and was allowed to stir for 1 h. Upon completion, the reaction contents were concentrated, then subsequently diluted with EtOAc (10 mL) and H₂O, extracted three times with EtOAc (10 mL), dried over Na₂SO₄, and concentrated in vacuo. The resulting crude residue was purified by flash column chromatography to provide the desired product. LCMS calculated for C₁₂H₁₀F₃N₂O₂ (M+H)⁺: m/z=271.1; found 271.1.

### Step 8. 2-(2-Amino-8-(trifluoromethyl)quinolin-5-yl)propan-2-ol

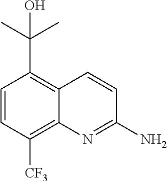 [SMILES: CC(C)(O)c1ccc(C(F)(F)F)c2nc(N)ccc12]

To a solution of methyl 2-amino-8-(trifluoromethyl)quinoline-5-carboxylate (62 mg, 0.229 mmol) in THF (2.3 mL) at 0° C. was added methylmagnesium bromide (0.38 mL, 1.147 mmol). The reaction contents were stirred for 5 min. Upon completion, the reaction contents were quenched by the addition of H₂O (5 mL), extracted three times with EtOAc (10 mL), dried over Na₂SO₄, and concentrated in vacuo. The crude material was purified by flash column chromatography to provide the desired product. LCMS calculated for C₁₃H₁₄F₃N₂O (M+H)⁺: m/z=271.1; found 271.2.

### Step 9. Methyl ((1R,3R)-3-(6-((5-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

To a vial containing methyl ((1R,3R)-3-(6-chloro-3-(methyl-d₃)-2-oxo-2,3-dihydro-1H-imidazo[4,5-c]pyridin-1-yl)cyclopentyl)carbamate (19 mg, 0.056 mmol), 2-(2-amino-8-(trifluoromethyl)quinolin-5-yl)propan-2-ol (31 mg, 0.113 mmol), Pd₂(dba)₃ (8 mg, 8.5 μmol), Xantphos (10 mg, 0.017 mmol) and Cs₂CO₃ (37 mg, 0.113 mmol) was added 1,4-dioxane (1.1 mL). The reaction was degassed with N₂ for 10 min. The reaction was then heated to 100° C. and was allowed to stir for 16 h. Upon completion, the reaction contents were diluted with acetonitrile (4 mL) and purified by prep-LCMS (Xbridge C18 column, eluting with a gradient of acetonitrile/water containing 0.1% TFA, at flow rate of 60 mL/min. LCMS calculated for C₂₇H₂₇D₃F₃N₆O₄ (M+H)⁺: m/z=562.2; found: 562.3.

### Example A. JAK2 LanthaScreen JH1 Binding Assay

JAK2 JH1 binding assay utilizes catalytic domain (JH1, amino acids 826-1132) of human JAK2 expressed as N-terminal FLAG-tagged, biotinylated protein in a baculovirus expression system (Carna Biosciences, Product #08-445-20N). The assay was conducted in black 384-well polystyrene plates in a final reaction volume of 20 μL. JAK2 JH1 (1.5 nM) was incubated with compounds (100 nL serially diluted in DMSO) in the presence of 50 nM fluorescent JAK2-JH1 tracer and 0.5 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 2 hours at 25° C., LanthaScreen signals were read on a PHERAstar FS plate reader (BMG LABTECH). Data was analyzed with IDBS Xlfit and GraphPad Prism 5.0 software using a four parameter dose response curve to determine IC50 for each compound.

### Example B. JAK2 LanthaScreen JH2-WT Binding Assay

JAK2 JH2-WT binding assay utilizes pseudo-kinase domain (JH2, amino-acids 536-812 with 3 surface mutations W659A, W777A, F₇₉₄H) of human Wild Type JAK2 expressed as C-terminal His-Avi-tagged, biotinylated protein in a baculovirus expression system (BPS Bioscience, Catalog #79463). The assay was conducted in black 384-well polystyrene plates in a final reaction volume of 20 μL. JAK2 JH2-WT (0.145 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS Xlfit and GraphPad Prism 5.0 software using a four parameter dose response curve to determine IC50 for each compound.

### Example C. JAK2 LanthaScreen JH2-V617F Binding Assay

JAK2 JH2-V617F binding assay utilizes pseudo-kinase domain (JH2, amino-acids 536-812 with 3 surface mutations W659A, W777A, F₇₉₄H) of human V617F mutant JAK2 expressed as C-terminal His-Avi-tagged, biotinylated protein in a baculovirus expression system (BPS Bioscience, Catalog #79498). The assay was conducted in black 384-well polystyrene plates in a final reaction volume of 20 μL. JAK2 JH2-V617F (0.26 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS Xlfit and GraphPad Prism 5.0 software using a four parameter dose response curve to determine IC50 for each compound.

### Example D. JAK2 HTRF Enzyme Activity Assay

JAK2 enzyme activity assays utilize catalytic domain (JH1, amino acids 808-1132) of human JAK2 expressed as N-terminal His-tagged protein in a baculovirus expression system (BPS Bioscience, Catalog #40450). The assays was conducted in black 384-well polystyrene plates in a final reaction volume of 20 μL. JAK2 (0.015 nM) was incubated with compounds (100 nL serially diluted in DMSO) in the presence of ATP (30 μM or 1 mM) and 500 nM Biotin-labeled EQEDEPEGDYFEWLE (SEQ ID NO.: 1) peptide (BioSource International, custom synthesis) in assay buffer (50 mM Tris, pH=7.5, 10 mM MgCl₂, 0.01% Brij-35, 0.1% BSA, 1 mM EGTA, 5% Glycerol and 5 mM DTT) for 60 minutes at 25° C. The reactions were stopped by the addition of 10 μL of detection buffer (50 mM Tris, pH 7.8, 0.5 mg/mL BSA, 150 mM NaCl), supplemented with EDTA, LANCE Eu-W1024 anti-phosphotyrosine (PY20), (PerkinElmer, Catalog #AD0067) and Streptavidin SureLight APC (PerkinElmer Catalog #CR130-100), for a final concentration of 15 mM, 1.5 nM and 75 nM, respectively. HTRF signals were read after 30 minutes incubation at room temperature on a PHERAstar FS plate reader (BMG LABTECH). Data was analyzed with TDBS Xlfit and GraphPad Prism 5.0 software using a four parameter dose response curve to determine IC50 for each compound.

The compounds of the disclosure were tested in one or more of the assays described in Examples A-D, and the resulting data are shown in Table A.

TABLE A

| | | JH2 BIND | JH2 BIND | |
|---|---|---|---|---|
| | JH1 BIND | WT | V617F | JAK2 HTRF |
| Example | (EX. A) | (EX. B) | (Ex. C) | (Ex. D) |
| 1 | +++++ | + | + | +++++ |
| 2 | +++++ | + | + | +++++ |
| 3 | +++++ | + | + | +++++ |
| 4 | +++++ | + | + | +++++ |
| 5 | +++++ | + | + | +++++ |
| 6 | +++++ | + | + | +++++ |
| 7 | +++++ | + | ++ | +++++ |
| 8 | +++++ | ++ | ++ | +++++ |
| 9 | +++++ | + | + | +++++ |
| 10 | +++++ | + | + | +++++ |
| 11 | | | + | +++++ |
| 12 | | | + | +++++ |
| 13 | | | + | +++++ |
| 14 | | | + | +++++ |
| 15 | +++ | + | + | +++ |
| 16 | +++ | + | + | +++ |
| 17 | +++++ | + | + | +++++ |
| 18 | ++++ | + | + | ++++ |
| 19 | +++++ | + | + | +++++ |
| 20 | +++++ | + | + | +++++ |
| 21 | +++++ | + | + | +++++ |
| 22 | +++++ | + | + | +++++ |
| 23 | +++++ | ++ | ++ | +++++ |
| 24 | +++++ | + | + | +++++ |
| 25 | +++++ | + | + | +++++ |
| 26 | +++++ | + | + | +++++ |
| 27 | +++++ | + | + | +++++ |
| 28 | +++++ | + | + | +++++ |
| 29 | +++++ | + | + | +++++ |
| 30 | +++++ | + | + | +++++ |
| 31 | +++++ | + | + | +++++ |
| 32 | | | + | +++++ |
| 33 | | | ++ | +++++ |
| 34 | | | ++ | +++++ |
| 35 | +++++ | + | + | +++++ |
| 36 | +++++ | + | + | +++++ |
| 37 | +++ | + | ++ | +++ |
| 38 | +++++ | + | + | +++++ |
| 39 | +++++ | + | ++ | +++++ |
| 40 | +++++ | + | ++ | +++++ |
| 41 | +++++ | + | + | +++++ |
| 42 | +++++ | + | + | +++++ |
| 43 | +++++ | + | + | +++++ |
| 44 | +++++ | +++ | +++ | +++++ |
| 45 | +++++ | ++ | +++ | +++++ |
| 46 | | | + | +++++ |
| 47 | +++++ | + | + | +++++ |

+ refers to IC₅₀ of ≤10 nM
++ refers to IC₅₀ of >10 nM to ≤100 nM
+++ refers to IC₅₀ of >100 nM to ≤500 nM
++++ refers to IC₅₀ of >500 nM to ≤1000 nM
+++++ refers to IC₅₀ of >1000 nM

### Example E. Cell Culture and STAT5 (Tyr694) Phosphorylation Cell Based Assay

Ba/F3 cells expressing human JAK2 V617F/EPOR (mouse JAK2 WT knocked out by CRISPR) are cultured in RPMI media with 10% FBS, 1 μg/mL Puromycin, 1 mg/mL Geneticin (Thermo Fisher). Ba/F3 cells expressing human JAK2 WT/EPOR are cultured in RPMI media with 10% FBS, 1 μg/mL Puromycin, 1 mg/mL Geneticin and 2 ng/mL EPO. 24 hours before the assay, the culture medium for JAK2 V617F/EPOR Ba/F3 cells are changed to RPMI with 10% FBS without antibiotic (assay medium 1). Culture medium for Ba/F3 cells expressing human JAK2 WT/EPOR are changed to RPMI with 10% FBS and 2 ng/mL EPO (R&D systems) without antibiotic (assay medium 2). 50 nL/well test compounds in DMSO are transferred to the 384 white low volume cell culture plate (Greiner Bio-one) by ECHO liquid handler (Labcyte). The cells are centrifuged, resuspended in the corresponding fresh assay medium and dispensed at 10 μL/well (6×10⁶ cells/mL) with 0.5% DMSO in the final assay. After the treated cells are incubated at 37° C., 5% CO₂ for 2 hours, 4 μL/well supplemented lysis buffer (100× blocking buffer diluted 25 fold in 4× lysis buffer, Perkin-Elmer) are added and incubated at room temperature for 60 min with gentle shaking on orbital shaker at 600 rpm. Phospho-STAT5 Cryptate antibody and Phospho-STAT5 d2 antibody (1:1 vol/vol, Perkin-Elmer) are premixed and diluted 20 fold within the detection buffer. 4 μL of the premixed antibody solution are added to each well followed with 16 hours incubation at room temperature. The product activity is determined by measuring the fluorescence at 620 nm and 665 nm on Pherastar microplate reader (BMG Labtech). A ratio is calculated (665/620 nm) for each well. Wells with DMSO serve as the positive controls and wells containing high concentration of control compound are used as negative controls. IC₅₀ determination is performed by fitting the curve of percent control activity versus the log of the compound concentration using the Genedata Screener software.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 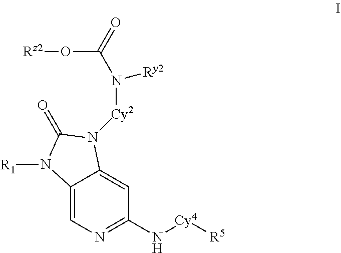
or a pharmaceutically acceptable salt thereof, wherein:
R¹ is selected from C₁₋₆ alkyl, C₂₋₆ alkenyl, and C₂₋₆ alkynyl, wherein the C₁₋₆ alkyl, C₂₋₆ alkenyl, and C₂₋₆ alkynyl of R¹ are each optionally substituted with 1, 2, or 3 independently selected R^{1A} substituents;
each R^{1A} is independently selected from halo, oxo, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b1}, S(O)₂NR^{c11}R^{d11}, OS(O)(═NR^{e11})R^{b11}, and OS(O)₂R^{b11};
each R^{a11}, R^{b11}, R^{c11}, and R^{d11} is independently selected from H, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{a11}, R^{b11}, R^{c11} and R^{d11} are each optionally substituted with 1, 2, 3, 4, 5, 6, 7, or 8 independently selected R^{M} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, and C₂₋₆ alkynyl;
Cy² is selected from C₃₋₁₀ cycloalkyl and 4-12 membered heterocycloalkyl, wherein the C₃₋₁₀ cycloalkyl and 4-12 membered heterocycloalkyl are each optionally substituted with 1, 2, 3, 4, 5, 6, 7 or 8 independently selected R² substituents;
each R² is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})R^{c2}R^{d2}, NR^{c2}C(═NR^{e2})R^{b2}, NR^{c2}S(O)R^{b2}, NR^{c2}S(O)NR^{c2}R^{d2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f2} and R^{g2} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{y2} is selected from H, C₁₋₆ alkyl, C₁₋₆ haloalkyl, C₂₋₆ alkenyl, and C₂₋₆ alkynyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, and (4-10 membered heterocycloalkyl)-C₁₋₆ alkyl- of R^{z2} are each optionally substituted with 1, 2, 3, 4, 5, 6, 7, or 8 independently selected R^{2A} substituents;
each R^{2A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})R^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21} S(O)R^{b21}, S(O)NR^{c21}R^{d21}, S(O)₂R^{b21}, S(O)₂NR^{c21}R^{d21}, OS(O)(═NR^{e21})R^{b21} OS(O)₂R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f21} and R^{g21} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h21} and Rⁱ²¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)R^{b22}, NR^{c22}S(O)NR^{c22}R^{d22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22})R^{b22}, and OS(O)₂R^{b22}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{2C} are each optionally substituted with 1, 2, 3, or 4 independently selected R^{M} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a22}, R^{c22} and R^{d22} are each optionally substituted with 1, 2, 3, or 4 independently selected R^{M} substituents;
or, any R^{c22} and R^{d22} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b22} are each optionally substituted with 1, 2, 3, or 4 independently selected R^{M} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R³ is selected from H, halo, C₁₋₆ alkyl, C₂₋₆ alkenyl, and C₂₋₆ alkynyl;
Cy⁴ is selected from 8-11 membered bicyclic heteroaryl, wherein the 8-11 membered bicyclic heteroaryl is optionally substituted with 1, 2, 3, 4, 5, 6, 7 or 8 independently selected R⁴ substituents;
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, (5-10 membered heteroaryl)-C₁₋₆ alkyl-, CN, NO₂, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4} S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R⁴ are each optionally substituted with 1, 2, 3, 4, 5, 6, 7, or 8 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{a4}, R^{c4} and R^{d4} are each optionally substituted with 1, 2, 3, 4, 5, 6, 7, or 8 independently selected R^{4A} substituents;
or, any R^{c4} and R^{d4} attached to the same N atom, together with the N atom to which they are attached, form a 5-10 membered heteroaryl, wherein the 5-10 membered heteroaryl is optionally substituted with 1, 2, 3, or 4 independently selected R^{4A} substituents;
each R^{b4} is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{b4} are each optionally substituted with 1, 2, 3, 4, 5, 6, 7, or 8 independently selected R^{4A} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{j4} and R^{k4} is independently selected from OH, C₁₋₆ alkoxy, and C₁₋₆ haloalkoxy;
or any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4A} is independently selected from halo, oxo, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, (5-10 membered heteroaryl)-C₁₋₆ alkyl-, CN, NO₂, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, SF₅, P(O)R^{f41}R^{g41}, OP(O)(OR^{h41})(ORⁱ⁴¹), P(O)(OR^{h41})(ORⁱ⁴¹), and BR^{j41}R^{k41}, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{4A} are each optionally substituted with 1, 2, 3, 4, 5, 6, 7, or 8 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{a41}, R^{c41} and R^{d41} are each optionally substituted with 1, 2, 3, 4, 5, 6, 7, or 8 independently selected R^{4B} substituents;
or, any R^{c41} and R^{d41} attached to the same N atom, together with the N atom to which they are attached, form a 5-10 membered heteroaryl, wherein the 5-10 membered heteroaryl is optionally substituted with 1, 2, 3, or 4 independently selected R^{4B} substituents;
each R^{b41} is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-, wherein the C₁₋₆ alkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl- of R^{b41} are each optionally substituted with 1, 2, 3, 4, 5, 6, 7, or 8 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{f41} and R^{g41} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{h41} and Rⁱ⁴¹ is independently selected from H, C₁₋₆ alkyl, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-;
each R^{j41} and R^{k41} is independently selected from OH, C₁₋₆ alkoxy, and C₁₋₆ haloalkoxy;
or any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from halo, oxo, C₁₋₆ alkyl, C₁₋₆ haloalkyl, C₂₋₆ alkenyl, C₂₋₆ alkynyl, phenyl, 5-6 membered heteroaryl, phenyl-C₁₆ alkyl-, (5-6 membered heteroaryl)-C₁₋₆ alkyl-, CN, NO₂, OR^{a42}, SR^{a42}, NHOR^{a42}, C(O)R^{b42}, C(O)NR^{c42}R^{d42}, C(O)NR^{c42}(OR^{a42}) C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)R^{b42}, NR^{c42}S(O)NR^{c42}R^{d42}, NR^{c42}S(O)₂R^{b42}, NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, and OS(O)₂R^{b42}, wherein the C₁₋₆ alkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl- of R^{4B} are each optionally substituted with 1, 2, 3, or 4 independently selected R^{P} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl-, wherein the C₁₋₆ alkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl- of R^{a42}, R^{c42} and R^{d42} are each optionally substituted with 1, 2, 3, or 4 independently selected R^{P} substituents;
or, any R^{c42} and R^{d42} attached to the same N atom, together with the N atom to which they are attached, form a 5-6 membered heteroaryl, wherein the 5-6 membered heteroaryl is optionally substituted with 1, 2, 3, or 4 independently selected R^{P} substituents;
each R^{b42} is independently selected from H, C₁₋₆ alkyl, C₁₋₆ haloalkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl-, wherein the C₁₋₆ alkyl, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl- of R^{b42} are each optionally substituted with 1, 2, 3, or 4 independently selected R^{P} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, 5-6 membered heteroaryl, phenyl-C₁₋₆ alkyl-, and (5-6 membered heteroaryl)-C₁₋₆ alkyl-;
R⁵ is selected from —C₁₋₆ alkyl-(R^{a51})ₚ, halo, CN, NO₂, oxo, OR^{b51}, SR^{b51}, NR^{c51}R^{b51}, OC(O)R^{b51}, OC(NR^{c51})R^{b51}, OC(O)NR^{c51}R^{b51}, NR^{c51}C(O)R^{b51}, NR^{c51}C(O)OR^{b51}, NR^{c51}C(O)NR^{c51}R^{b51}, NR^{c51}SO₂R^{b51}, NR^{c51}SO₂NR^{c51}R^{b51}, NR^{c51}S(O)R^{b51}, C(O)OR^{b51}, C(O)R^{b51}, C(O)NR^{c51}R^{b51}, S(O)R^{b51}, S(O)NR^{c51}R^{b51}, S(O)₂R^{b51}, S(O)(NH)R^{b51}, and SO₂NR^{c51}R^{b51};
p is 0, 1, 2, 3, 4, 5, or 6;
each R^{a51} is independently selected from halo, CN, NO₂, oxo, OR^{a52}, NHR^{a52}, NR^{b52}R^{a52}, SR^{a52}, SO₂R^{a52}, C(O)R^{a52}, C(O)OR^{a52}, C(O)NHR^{a52}, C(O)NR^{b52}R^{a52}, OC(O)R^{a52}, S(O)R^{a52}, S(O)NR^{a52}R^{a52}, S(O)₂R^{a52}, and SO₂NR^{b52}R^{a52};
each R^{b51} and R^{c51} are independently selected from H, C₁₋₆ alkyl, and C₁₋₆ haloalkyl;
each R^{a52} and R^{b52} are independently selected from H, and C₁₋₆ alkyl;
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-; and
each R^{P} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, 5-10 membered heteroaryl, C₆₋₁₀ aryl-C₁₋₆ alkyl-, and (5-10 membered heteroaryl)-C₁₋₆ alkyl-.

2. The compound of claim 1, or a pharmaceutically acceptable salt thereof, wherein R¹ is C₁₋₆ alkyl.

3. (canceled)

4. The compound of claim 1, or a pharmaceutically acceptable salt thereof, wherein R¹ is selected from methyl and trideuteromethyl.

5. The compound of claim 1, or a pharmaceutically acceptable salt thereof, wherein Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents.

6. (canceled)

7. The compound of claim 1, or a pharmaceutically acceptable salt thereof, wherein Cy² is selected from cyclobutyl, cyclopentyl, deuterocyclopentyl, bicyclo[1.1.1]pentanyl, and bicyclo[2.1.1]hexanyl, wherein the cyclobutyl, cyclopentyl, deuterocyclopentyl, bicyclo[1.1.1]pentanyl, and bicyclo[2.1.1]hexanyl of Cy² are each optionally substituted with 1, 2, 3, or 4 independently selected R² substituents.

8. The compound of claim 1, or a pharmaceutically acceptable salt thereof, wherein each R² is independently selected from halo, oxo, C₁₋₆ alkyl, C₁₋₆ haloalkyl, C₂₋₆ alkenyl, and C₂₋₆ alkynyl.

9. (canceled)

10. The compound of claim 1, or a pharmaceutically acceptable salt thereof, wherein each R² is methyl.

11. The compound of claim 1, or a pharmaceutically acceptable salt thereof, wherein Cy² is selected from cyclobutyl, cyclopentyl, deuterocyclopentyl, methylcyclopentyl, bicyclo[1.1.1]pentanyl, and bicyclo[2.1.1]hexanyl.

12. The compound of claim 1, or a pharmaceutically acceptable salt thereof, wherein R^{y2} is selected from H and C₁₋₆ alkyl.

13. (canceled)

14. The compound of claim 1, or a pharmaceutically acceptable salt thereof, wherein R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents.

15. (canceled)

16. The compound of claim 1, or a pharmaceutically acceptable salt thereof, wherein R^{z2} is selected from methyl, ethyl, cyclopropyl, and phenylmethyl, wherein the methyl, ethyl, cyclopropyl, and phenylmethyl of R^{z2} are each optionally substituted with 1, 2, 3, or 4 independently selected R^{2A} substituents.

17. The compound of claim 14, or a pharmaceutically acceptable salt thereof, wherein each R^{2A} is independently selected from C₁₋₆ alkyl, C₁₋₆ haloalkyl, C₂₋₆ alkenyl, C₂₋₆ alkynyl, and OR^{a21}; and
each R^{a21} is independently selected from H and C₁₋₆ alkyl.

18. (canceled)

19. The compound of claim 14, or a pharmaceutically acceptable salt thereof, wherein each R^{2A} is methoxy.

20. The compound of claim 1, or a pharmaceutically acceptable salt thereof, wherein R^{z2} is selected from methyl, methoxyethyl, cyclopropyl, and phenylmethyl.

21. The compound of claim 1, or a pharmaceutically acceptable salt thereof, wherein R³ is selected from H and C₁₋₆ alkyl.

22-23. (canceled)

24. The compound of claim 1, or a pharmaceutically acceptable salt thereof, wherein Cy⁴ is selected from quinolinyl, naphthyridinyl, quinoxalinyl, and imidazo[1,2-b]pyridazinyl, wherein the quinolinyl, naphthyridinyl, quinoxalinyl, and imidazo[1,2-b]pyridazinyl are each optionally substituted with 1, 2, 3, or 4 independently selected R⁴ substituents.

25. The compound of claim 1, or a pharmaceutically acceptable salt thereof, wherein each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4};
each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents.

26. (canceled)

27. The compound of claim 1, or a pharmaceutically acceptable salt thereof, wherein each R⁴ is independently selected from methyl, isopropyl, trifluoromethyl, and NHC(O)R^{b4}, wherein each methyl and isopropyl of R⁴ is optionally substituted with 1 or 2 independently selected R^{4A} substituents;
each R^{b4} is independently selected from H and C₁₋₆ alkyl, wherein each C₁₋₆ alkyl of R^{b4} is optionally substituted with 1 or 2 independently selected R^{4A} substituents.

28. The compound of claim 1, or a pharmaceutically acceptable salt thereof, wherein each R^{4A} is independently selected from C₁₋₆ alkyl, C₁₋₆ haloalkyl, C₂₋₆ alkenyl, C₂₋₆ alkynyl, and OR^{a41}; and
each R^{a41} is independently selected from H and C₁₋₆ alkyl.

29. (canceled)

30. The compound of claim 1, or a pharmaceutically acceptable salt thereof, wherein each R^{4A} is independently selected from methyl and hydroxy.

31. The compound of claim 1, or a pharmaceutically acceptable salt thereof, wherein each R⁴ is independently selected from methyl, hydroxymethyl, hydroxyisopropyl, trifluoromethyl, and NHC(O)CH₂OCH₃.

32. (canceled)

33. The compound of claim 1, or a pharmaceutically acceptable salt thereof, wherein R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51};
p is 0, 1, 2, 3, or 4;
each R^{a51} is independently selected from halo, CN, and OR^{a52};
R^{b51} and R^{c51} are each independently selected from H and C₁₋₆ alkyl; and
R^{a52} is selected from H and C₁₋₆ alkyl.

34-35. (canceled)

36. The compound of claim 1, or a pharmaceutically acceptable salt thereof, wherein R⁵ is selected from methyl, difluoromethyl, trifluoromethyl, (trifluoromethyl)ethyl, (trifluoromethyl)(hydroxy)ethyl, hydroxymethyl, cyanomethyl, hydroxyethyl, hydroxyisopropyl, methoxyisopropyl, cyanoisopropyl, fluoro, chloro, bromo, iodo, cyano, nitro, S(O)CH₃, S(O)₂CH₃, S(O)(NH)CH₃, C(O)N(CH₃)(CH(CH₃)₂), and —NHS(O)₂CH₃.

37. The compound of claim 1, or a pharmaceutically acceptable salt thereof, wherein:
R¹ is C₁₋₆ alkyl;
Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents;
each R² is independently selected from halo, oxo, C₁₋₆ alkyl, C₁₋₆ haloalkyl, C₂₋₆ alkenyl, and C₂₋₆ alkynyl;
R^{y2} is selected from H and C₁₋₆ alkyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents;
each R^{2A} is independently selected from C₁₋₆ alkyl, C₁₋₆ haloalkyl, C₂₋₆ alkenyl, C₂₋₆ alkynyl, and OR^{a21};
each R^{a21} is independently selected from H and C₁₋₆ alkyl;
R³ is selected from H and C₁₋₆ alkyl;
Cy⁴ is selected from 8-11 membered bicyclic heteroaryl, wherein the 8-11 membered bicyclic heteroaryl is optionally substituted with 1, 2, 3, 4, 5, 6, 7 or 8 independently selected R⁴ substituents;
each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4};
each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents;
each R^{4A} is independently selected from C₁₋₆ alkyl, C₁₋₆ haloalkyl, C₂₋₆ alkenyl, C₂₋₆ alkynyl, and OR^{a41};
each R^{a41} is independently selected from H and C₁₋₆ alkyl;
R⁵ is selected from —C₁₋₆ alkyl-(R^{a51})ₚ, halo, CN, NO₂, oxo, OR^{b51}, SR^{b51}, NR^{c51}R^{b51}, OC(O)R^{b51}, OC(NR^{c51})R^{b51}, OC(O)NR^{c51}R^{b51}, NR^{c51}C(O)R^{b51}, NR^{c51}C(O)OR^{b51}, NR^{c51}C(O)NR^{c51}R^{b51}, NR^{c51}SO₂R^{b51}, NR^{c51}SO₂NR^{c51}R^{b51}, NR^{c51}S(O)R^{b51}, C(O)OR^{b51}, C(O)R^{b51}, C(O)NR^{c51}R^{b51}, S(O)R^{b51}, S(O)NR^{c51}R^{b51}, S(O)₂R^{b51}, S(O)(NH)R^{b51}, and SO₂NR^{c51}R^{b51};
p is 0, 1, 2, 3, or 4;
each R^{a51} is independently selected from halo, CN, NO₂, oxo, OR^{a52}, NHR^{a51}, NR^{b52}R^{a52}, SR^{a52}, SO₂R^{a52}, C(O)R^{a52}, C(O)OR^{a52}, C(O)NHR^{a52}, C(O)NR^{b52}R^{a52}, OC(O)R^{a52}, S(O)R^{a52}, S(O)NR^{b52}R^{a52}, S(O)₂R^{a52}, and SO₂NR^{b52}R^{a52};
each R^{b51} and R^{c51} are independently selected from H, C₁₋₆ alkyl, and C₁₋₆ haloalkyl; and
each R^{a52} and R^{b52} are independently selected from H and C₁₋₆ alkyl.

38. The compound of claim 1, or a pharmaceutically acceptable salt thereof, wherein:
R¹ is C₁₋₆ alkyl;
Cy² is selected from C₃₋₁₀ cycloalkyl and 4-7 membered heterocycloalkyl, wherein the C₃₋₁₀ cycloalkyl and 4-7 membered heterocycloalkyl are each optionally substituted with 1, 2, 3, or 4 independently selected R² substituents;
each R² is independently selected from halo, oxo, C₁₋₆ alkyl, C₁₋₆ haloalkyl, C₂₋₆ alkenyl, and C₂₋₆ alkynyl;
R^{y2} is selected from H and C₁₋₆ alkyl;
R^{z2} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₆ alkyl-, and C₃₋₁₀ cycloalkyl-C₁₋₆ alkyl- of R^{z2} are each optionally substituted with 1, 2, 3, or 4 independently selected R^{2A} substituents;
each R^{2A} is independently selected from C₁₋₆ alkyl, C₁₋₆ haloalkyl, C₂₋₆ alkenyl, C₂₋₆ alkynyl, and OR^{a21};
each R^{a21} is independently selected from H and C₁₋₆ alkyl;
R³ is selected from H and C₁₋₆ alkyl;
Cy⁴ is a 9-10 membered bicyclic heteroaryl, wherein the 9-10 membered bicyclic heteroaryl is optionally substituted with 1, 2, 3, or 4 independently selected R⁴ substituents;
each R⁴ is independently selected from halo, C₁₋₆ alkyl, C₁₋₆ haloalkyl, C₂₋₆ alkenyl, C₂₋₆ alkynyl, CN, and NR^{c4}C(O)R^{b4};
each R^{b4} and R^{c4} is independently selected from H and C₁₋₆ alkyl, wherein the C₁₋₆ alkyl of R^{b4} and R^{c4} are each optionally substituted with 1, 2, 3, or 4 independently selected R^{4A} substituents;
each R^{4A} is independently selected from C₁₋₆ alkyl, C₁₋₆ haloalkyl, C₂₋₆ alkenyl, C₂₋₆ alkynyl, and OR^{a41};
each R^{a41} is independently selected from H and C₁₋₆ alkyl;
R⁵ is selected from —C₁₋₆ alkyl-(R^{a51})ₚ, halo, CN, NO₂, NR^{c51}C(O)R^{b51}, NR^{c51}SO₂R^{b51}, NR^{c51}S(O)R^{b51}, C(O)NR^{c51}R^{b51}, S(O)R^{b51}, S(O)₂R^{b51}, and S(O)(NH)R^{b51};
p is 0, 1, 2, 3, or 4;
each R^{a51} is independently selected from halo, CN, NO₂, oxo, OR^{a52}, NHR^{a52}, NR^{b52}R^{a52}, SR^{a52}, SO₂R^{a52}, C(O)R^{a52}, C(O)OR^{a52}, C(O)NHR^{a52}, C(O)NR^{b52}R^{a52}, OC(O)R^{a52}, S(O)R^{a52}, S(O)NR^{b52}R^{a52}, S(O)₂R^{a52}, and SO₂NR^{b52}R^{a52};
each R^{b51} and R^{c51} are independently selected from H, C₁₋₆ alkyl, and C₁₋₆ haloalkyl; and
each R^{a52} and R^{b52} are independently selected from H and C₁₋₆ alkyl.

39. The compound of claim 1, wherein the compound of Formula I is a compound of Formula II, Formula III, Formula IV, Formula V, or Formula VI: 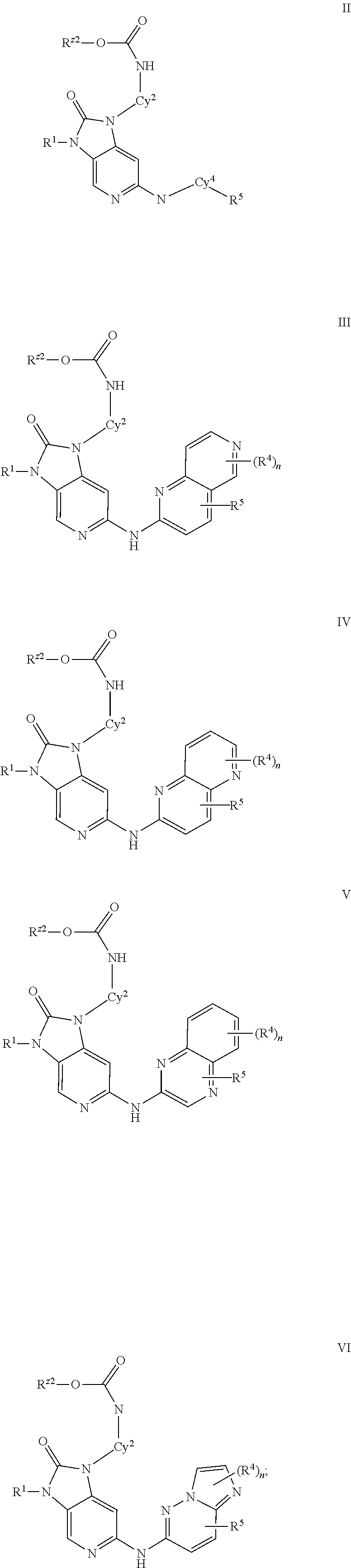
or a pharmaceutically acceptable salt thereof, wherein n is 0, 1, 2, or 3.

40-43. (canceled)

44. The compound of claim 1, which is selected from:
methyl ((1R,3R)-3-(6-((4-(hydroxymethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(1-hydroxyethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-2-oxo-6-((4-(1,1,1-trifluoro-2-hydroxypropan-2-yl)-8-(trifluoromethyl)quinolin-2-yl)amino)-2,3-dihydro-1H-imidazo[4,5-c]pyridin-1-yl)cyclopentyl)carbamate;
methyl ((1R,3R)-3-(3-(methyl-d₃)-6-((4-methyl-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(3-(methyl-d₃)-6-((4-(methylsulfinyl)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(3-(methyl-d₃)-6-((4-(methylsulfonyl)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(3-(methyl-d₃)-6-((4-(S-methylsulfonimidoyl)-8-(trifluoromethyl)quinolin-2-yl)amino)-2-oxo-2,3-dihydro-1H-imidazo[4,5-c]pyridin-1-yl)cyclopentyl)carbamate;
benzyl ((1R,3R)-3-(3-(methyl-d₃)-2-oxo-6-((8-(trifluoromethyl)quinolin-2-yl)amino)-2,3-dihydro-1H-imidazo[4,5-c]pyridin-1-yl)cyclopentyl)carbamate;
methyl ((1R,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(isopropyl(methyl)carbamoyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate;
2-methoxyethyl ((1S,3R)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)-1-methylcyclopentyl)carbamate;
methyl ((trans)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclobutyl)carbamate;
cyclopropyl ((trans)-3-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclobutyl)carbamate;
methyl ((1R,3R)-3-(6-((4-(2-methoxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(cyanomethyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-cyanopropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-chloro-4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-chloro-8-(trifluoromethyl)quinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((4-(2-methoxyacetamido)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-6-((4-(methylsulfonamido)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(6-((8-bromoquinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-chloro-8-(trifluoromethyl)quinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-cyano-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-fluoro-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-6-((8-methyl-1,6-naphthyridin-2-yl)amino)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-8-methyl-1,5-naphthyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-2-oxo-6-((3-(1,1,1-trifluoropropan-2-yl)imidazo[1,2-b]pyridazin-6-yl)amino)-2,3-dihydro-1H-imidazo[4,5-c]pyridin-1-yl)cyclopentyl)carbamate;
methyl ((1R,3R)-3-(3-(methyl-d₃)-2-oxo-6-((3-(trifluoromethyl)imidazo[1,2-b]pyridazin-6-yl)amino)-2,3-dihydro-1H-imidazo[4,5-c]pyridin-1-yl)cyclopentyl)carbamate;
methyl (4-(6-((4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)bicyclo[2.1.1]hexan-1-yl)carbamate;
methyl (3-(3-(methyl-d₃)-2-oxo-6-((8-(trifluoromethyl)quinolin-2-yl)amino)-2,3-dihydro-1H-imidazo[4,5-c]pyridin-1-yl)bicyclo[1.1.1]pentan-1-yl)carbamate;
methyl ((1R,3R)-3-(6-((8-(2-cyanopropan-2-yl)-4-(2-hydroxypropan-2-yl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-6-((8-nitroquinolin-2-yl)amino)-2-oxo-2,3-dihydro-1H-imidazo[4,5-c]pyridin-1-yl)cyclopentyl)carbamate;
methyl ((1R,3R)-3-(6-((8-cyano-1,5-naphthyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((8-cyanoquinoxalin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((8-(difluoromethyl)quinolin-2-yl)amino)-3-methyl-2-oxo-2,3-dihydro-1H-imidazo[4,5-c]pyridin-1-yl)cyclopentyl)carbamate;
methyl ((1R,3R)-3-(3-methyl-6-((8-methylquinolin-2-yl)amino)-2-oxo-2,3-dihydro-1H-imidazo[4,5-c]pyridin-1-yl)cyclopentyl)carbamate;
methyl ((1S,3S)-3-(6-((8-chloroquinolin-2-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((8-(trifluoromethyl)quinolin-2-yl)amino)-2,3-dihydro-1H-imidazo[4,5-c]pyridin-1-yl)cyclopentyl)carbamate;
methyl ((1R,3R)-3-(6-((3-cyanoimidazo[1,2-b]pyridazin-6-yl)amino)-3-methyl-2-oxo-2,3-dihydro-1H-imidazo[4,5-c]pyridin-1-yl)cyclopentyl)carbamate;
methyl ((1R,3R)-3-(6-((8-cyanoquinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((7-cyanoquinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-cyanoquinolin-2-yl)amino)-3-methyl-2-oxo-2,3-dihydro-1H-imidazo[4,5-c]pyridin-1-yl)cyclopentyl)carbamate;
methyl ((1R,3R)-3-(6-((3-iodo-2,8-dimethylimidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate; and
methyl ((1R,3R)-3-(6-((5-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
or a pharmaceutically acceptable salt thereof.

45. The compound of claim 1, or a pharmaceutically acceptable salt thereof, wherein the compound is deuterated.

46. A pharmaceutical composition, comprising a compound of claim 1, or a pharmaceutically acceptable salt thereof, and a pharmaceutically acceptable carrier.

47. (canceled)

48. A method of treating cancer in a patient in need thereof, the method comprising administering to the patient a therapeutically effective amount of a compound of claim 1, or a pharmaceutically acceptable salt thereof.

49-51. (canceled)

52. A method of treating a myeloproliferative disorder in a patient in need thereof, the method comprising administering to the patient a therapeutically effective amount of a compound of claim 1, or a pharmaceutically acceptable salt thereof.

53. (canceled)

54. A method of treating myelodysplastic syndrome in a patient in need thereof, the method comprising administering to the patient a therapeutically effective amount of a compound of claim 1, or a pharmaceutically acceptable salt thereof.
